(19) United States
(12) Patent Application Publication
(10) Pub. No.: US 2024/0238379 A1
(43) Pub. Date: Jul. 18, 2024

(54) Compositions and Methods for the Prevention and Treatment of Stress-Induced Fear, Depressive-Like and Anxiety-Like Behavior

(71) Applicant: The Trustees of Columbia University in the City of New York, New York, NY (US)

(72) Inventors: Christine A. Denny, Ho-Ho-Kus, NJ (US); Briana K. CHEN, New York, NY (US)

(73) Assignees: The Trustees of Columbia University in the City of New York, New York, NY (US); The Research Foundation for Mental Hygiene, Inc., Menands, NY (US)

(21) Appl. No.: 18/400,120

(22) Filed: Dec. 29, 2023

Related U.S. Application Data
(63) Continuation of application No. PCT/US2022/035725, filed on Jun. 30, 2022.
(60) Provisional application No. 63/279,127, filed on Nov. 14, 2021.
(60) Provisional application No. 63/216,743, filed on Jun. 30, 2021.

(51) Int. Cl. A61K 38/22; A61P 25/00; A61P 25/22

(52) U.S. Cl. CPC A61K 38/2278; A61P 25/00; A61P 25/22

(57) ABSTRACT

Compositions and methods for the prevention and treatment of stress-induced fear, depressive-like and anxiety-like behavior using selective VPAC2 agonist Bay 55-9837.

### CROSS-REFERENCE TO RELATED APPLICATIONS

Priority is claimed to U.S. Provisional Applications Nos. 63/216,743 filed 30 Jun. 2021 and 63/279,127 filed on 14 Nov. 2021, each of which is hereby incorporated by reference in its entirety.

### STATEMENT REGARDING FEDERALLY SPONSORED RESEARCH OR DEVELOPMENT

This invention was made with government support under MH121023 awarded by the National Institutes of Mental Health. The government has certain rights in the invention.

### TECHNICAL FIELD OF THE INVENTION

The present disclosure relates to the technical fields of neurobiology and behavioral disorders.

### BACKGROUND OF THE INVENTION

Exposure to stress is a major risk factor for mood and anxiety disorders, including major depressive disorder (MDD) and posttraumatic stress disorder (PTSD). Therapeutic intervention against stress could prevent the onset of mood and anxiety disorders and thereby reduce the associated healthcare burdens. These mental illnesses are a major global health challenge, affecting between 20-30% of adults in the United States alone. These disorders, which include PTSD and generalized anxiety disorder, are often comorbid with depression and cost in excess of $42 billion annually to treat. Regardless of age or socioeconomic status, women are twice as likely as men to be diagnosed with depression and PTSD. Moreover, women typically display different symptoms of these disorders than men and develop these illnesses earlier in life. The current paradigm for treating these disorders is to mitigate symptoms through medication or therapy after the disorder has already developed. However, this approach can be inefficient, costly, and ineffective in up to one-third of patients. Enhancing resilience to stress to prevent these disorders before they develop is therefore an innovative approach to significantly reduce the global burden of mood and anxiety disorders.

It has been previously demonstrated that several compounds can prevent behavioral despair and reduce hyponcophagia in females; however, to date, a drug is yet to be discovered that suppresses learned fear in female mice. Thus, there is a need for an improved protocol for preventing all three types of stress-related behaviors in both sexes.

Vasoactive intestinal peptide receptor 2 (VPAC2) is part of a larger family of receptors that bind the neuropeptides pituitary adenylate cyclase activating peptide (PACAP) or vasoactive intestinal peptide (VIP). This receptor family includes two other receptors, VPAC1 and PAC1, and activation of these receptors by either PACAP or VIP exerts a range of physiological effects on nervous, endocrine, cardiovascular, muscular, and immune systems. VPAC2 agonists have been proposed as a potential therapeutic for type II diabetes, spinal muscular atrophy, HIV/AIDS, and pulmonary hypertension, but have not been investigated for their potential in protecting subjects against stress-induced maladaptive behaviors. Surprisingly, the present inventors have discovered that low doses of the VPAC2-specific agonist, Bay 55-9837, provides such a protective effect against some or all stress-induced maladaptive behaviors.

### BRIEF SUMMARY OF THE INVENTION

Disclosed herein are prophylactic compositions and methods for protecting subjects against all three types of stress-induced maladaptive behaviors: fear, depressive-like behavior, and anxiety-like behavior. The disclosed compositions and methods can be used in a wide variety of stress-induced maladaptive behaviors including but not limited to major depressive disorder (MDD) and posttraumatic stress disorder (PTSD).

In certain embodiments, the disclosed methods are directed to preventing or delaying or treating a stress-induced affective disorder or stress-induced psychopathology in a subject, comprising administering to the subject a composition comprising a prophylactically effective amount of a vasoactive intestinal peptide receptor 2 (VPAC2) agonist, wherein administration of the composition prevents or delays at least one stress-induced maladaptive behavior in the subject selected from the group consisting of fear, depressive-like behavior, and anxiety-like behavior.

In other embodiments, the disclosed methods are directed to inducing and/or enhancing stress resilience in a subject, comprising administering to the subject a composition comprising a prophylactically effective amount of a vasoactive intestinal peptide receptor 2 (VPAC2) agonist, wherein administration of the composition induces and/or enhances stress resilience in the subject to at least one stress-induced maladaptive behavior selected from the group consisting of fear, depressive-like behavior, and anxiety-like behavior.

In other embodiments, the disclosed methods are directed to enhancing and facilitating contextual fear discrimination in a subject, comprising administering a composition comprising a prophylactically effective amount of aVPAC2 agonist to the subject.

In other embodiments, the disclosed methods are directed to reducing perseverative behavior in a subject, comprising administering a composition comprising a prophylactically effective amount of aVPAC2 agonist to the subject, wherein the subject has not been exposed to stress.

In other embodiments, the disclosed methods are directed to reducing anxiety in a subject, comprising administering a composition comprising a prophylactically effective amount of aVPAC2 agonist to the subject, wherein the subject has not been exposed to stress.

In certain embodiments, the composition is administered to the subject prior to a stressor. In certain embodiments, the composition or compositions are administered to the subject once after exposure to a stressor. In certain embodiments, the composition or compositions are administered in a booster series. In certain embodiments, the composition or compositions are administered at least once prior to the stressor and at least once after the stressor. In certain embodiments, the composition or compositions are administered orally, intravenously, intranasally, or via injection to the subject.

In certain embodiments, administration of the composition prevents or delays multiple stress-induced maladaptive behaviors in the subject. In certain embodiments, administration of the composition induces and/or enhances stress resilience in the subject against multiple stress-induced maladaptive behavior. In certain instances, all stress-induced maladaptive behaviors in the subject may be ameliorated.

In some embodiments, contemplated stress-induced affective disorders include but are not limited to major depressive disorder (MDD), posttraumatic stress disorder (PTSD), depressive-like behavior and associated affective disorders, anhedonic behavior and associated affective disorders, anxiety and associated affective disorders, cognitive impairments and deficits and associated disorders, stress-induced fear, and combinations thereof.

In some embodiments, the subject may be a mammal. In certain embodiments, the subject is a human. In particular embodiments, the prophylactic effect may be limited to a female subject or male subject.

In some embodiments, the VPAC2 agonist is BAY 55-9837. In particular embodiments, BAY 55-9837 is administered to the subject in an amount ranging from 2.5 mg/kg body weight of the subject to 10 mg/kg body weight of the subject.

### BRIEF DESCRIPTION OF THE SEVERAL VIEWS OF THE DRAWINGS

FIG. 1 shows that Bay 55-9837, a selective VPAC2 agonist, is prophylactic against stress in female mice. (A) Experimental design. Saline, (R,S)-ketamine (10 mg/kg), or Bay 55-9837 (2.5 or 5 mg/kg) was administered 1 week prior to 3-shock contextual fear conditioning (CFC) stress in female 129S6/SvEv mice. Five days later, mice were assayed for fear, behavioral despair, locomotion, exploratory behavior, and hyponcophagia. (B-C) Freezing was comparable across all groups during CFC training. (D-E) Bay 55-9837 (2.5 mg/kg), but not (R,S)-ketamine (10 mg/kg) or Bay 55-9837 (5 mg/kg), significantly reduced freezing during CFC re-exposure when compared with saline. (F-G) On FST Day 1, immobility time was significantly reduced in mice administered Bay 55-9837 (2.5 mg/kg), but not (R,S)-ketamine (10 mg/kg) or Bay 55-9837 (5 mg/kg). (H-I) On FST Day 2, both (R,S)-ketamine (10 mg/kg) and Bay 55-9837 (2.5 mg/kg), but not Bay 55-9837 (5 mg/kg), reduced behavioral despair. (J-K) In mice administered 2.5 and 5 mg/kg of Bay 5-9837, but not (R,S)-ketamine (10 mg/kg), latency to feed in the novel environment was significantly lowered when compared to saline controls. (L-M) Feeding in the home cage and weight loss following food deprivation was not significantly altered in experimental groups. (n=4-15 female mice). Error bars represent±SEM. * p<0.05, ** P<0.01, *** p<0.001, **** p<0.0001. CFC, contextual fear conditioning; FST, forced swim test; OF, open field; EPM, elevated plus maze; NSF, novelty suppressed feeding; min, minute; Sal, saline; K, (R,S)-ketamine; Bay, Bay 55-9837; mg, milligram; kg, kilogram; sec, second; HC, home cage; g, gram.

FIG. 2 shows that Bay 55-9837 reduces behavioral despair and hyponcophagia, but does not attenuate learned fear, in male mice. (A) Experimental design. Male 129S6/SvEv mice were administered the same behavioral paradigm as in FIG. 1A. (B-E) Freezing during CFC training and re-exposure was comparable across all groups. (F-G) There was no effect of drug on immobility time during FST Day 1. (H-I) However, on FST Day 2, Bay 55-9837 (5 and 10 mg/kg), but not at 2.5 or 7.5 mg/kg, significantly reduced immobility when compared with saline controls. (J-K) Bay 55-9837 (7.5 mg/kg), but no other dose, suppressed latency to feed in the OF during the NSF assay. (L) Latency to feed in the home cage was comparable across all groups. (M) Mice administered Bay 55-9837 (7.5 mg/kg) lost significantly more body weight when compared to saline-administered mice. Error bars represent±SEM. * p<0.05, ** P<0.01, *** p<0.001, **** p<0.0001. CFC, contextual fear conditioning; FST, forced swim test; OF, open field; EPM, elevated plus maze; NSF, novelty-suppressed feeding; min, minute; Sal, saline; Bay, Bay 55-9837; mg, milligram; kg, kilogram; sec, second; OF, open field; HC, home cage; g, gram.

FIG. 3 shows that Bay 55-9837 is prophylactic in female mice when administered after stress. (A) Experimental design. Female 129S6/SvEv mice were given a single injection of saline, (R,S)-ketamine (10 mg/kg), or Bay 55-9837 (2.5 mg/kg) 5 minutes after 3-shock CFC training. (B-E) Freezing during CFC training and re-exposure was not significantly altered by drug administration. (F-I) Both (R,S)-ketamine and Bay 55-9837 significantly reduced immobility time on Day 2, but not Day 1, of the FST. (J-K) Latency to feed in the open field was significantly decreased in mice administered Bay 55-9837, but not (R,S)-ketamine. (L) Latency to feed in the home cage and (M) body weight loss were comparable between all groups. Error bars represent±SEM. * p<0.05, ** P<0.01, *** p<0.001, **** p<0.0001. CFC, contextual fear conditioning; FST, forced swim test; OF, open field; EPM, elevated plus maze; NSF, novelty-suppressed feeding; min, minute; mg, milligram; kg, kilogram; Sal, saline; K, (R,S)-ketamine; Bay, Bay 55-9837; sec, second; HC, home cage; g, gram.

FIG. 4 shows that Bay 55-9837 is prophylactic in male mice when administered after stress. (A) Experimental design. Male 129S6/SvEv mice were administered the same behavioral paradigm as in FIG. 3A. (B-E) (R,S)-ketamine and Bay 55-9837 administration did not significantly alter freezing during CFC training or re-exposure. (F-I) As in female mice, both drugs tested significantly decreased immobility time on Day 2, but not Day 1, of the FST. (J-K) Similarly, in the NSF, Bay 55-9837, but not (R,S)-ketamine suppressed latency to feed in the OF when compared with saline controls. (L-M) Home cage feeding behavior and body weight loss were not significantly altered by drug administration. Error bars represent±SEM. * p<0.05, ** P<0.01, *** p<0.001, **** p<0.0001. CFC, contextual fear conditioning; FST, forced swim test; OF, open field; EPM, elevated plus maze; NSF, novelty-suppressed feeding; min, minute; mg, milligram; kg, kilogram; Sal, saline; K, (R,S)-ketamine; Bay, Bay 55-9837; sec, second; HC, home cage; g, gram.

FIG. 5 shows that Bay 55-9837 prevents stress-induced alterations in sleep/wake cycles in female mice. (A) Experimental protocol. Female 129S6/SEv mice were administered a single injection of saline of Bay 55-9837 (2.5 mg/kg) and placed in Piezo sleep boxes. One week later, mice were administered 3-shock CFC training or context exposure and again placed back into Piezo sleep boxes. Five days later, mice were re-exposed to the training context. (B) On Day 2 after injection, there was no significant difference in activity between saline controls and Bay 55-9837-administered mice. (C-D) Percentage of sleep and overall amplitude across Days 2-7 were comparable between saline and Bay 55-9837-administered mice. (E-F) During CFC training, there was no effect of Drug. Mice administered CFC froze significantly more on minute 5 when compared to mice given context exposure. (G-H) During CFC re-exposure, mice exposed to CFC froze significantly more when compared with mice exposed to context exposure. In the CFC-administered group, mice given Bay 55-9837 exhibited significantly less freezing behavior compared to saline-administered mice. (I) On Day 9, mice administered saline and exposed to CFC exhibited a significantly increased activity level at ZT12 when compared to non-stressed groups. Mice administered Bay 55-9837 prior to CFC had a significantly reduced activity level at ZT12 that was comparable to non-stressed mice. (J) Percentage of sleep on days 9-11 was comparable across all groups. (K) There was a significant effect of Drug and Stress as well as a Drug x Stress interaction. In the non-stressed mice, Bay 55-9837 did not significantly alter sleep/wake amplitude. Saline-administered mice exposed to CFC stress exhibited a significantly reduced amplitude when compared to non-stressed saline controls. Administration of Bay 55-9837 prior to stress significantly increase sleep/wake amplitude to a level comparable to non-stressed controls. Error bars represent±SEM. * p<0.05, ** P<0.01, *** p<0.001, **** p<0.0001. CFC, contextual fear conditioning; mg, milligram; kg, kilogram; ZT, Zeitgeiber time; Sal, saline; Bay, Bay 55-9837; min, minute.

FIG. 6 shows that Bay 55-9837 enhances and facilitates contextual fear discrimination in female, but not male mice. (A) Experimental paradigm. Mice were given a single injection of saline or Bay 55-9837 (2.5 mg/kg in female mice, 10 mg/kg in male mice) one week prior to contextual fear discrimination. (B) Saline-administered female mice began discriminating on Day 7 whereas (C) Bay 55-9837-administered mice began discriminating on Day 4. (D) Bay 55-9837 significantly increased fear discrimination in female mice when compared to saline controls. (E) By Day 5 of the behavioral paradigm, Bay 55-9837-administered, but not saline-administered, mice froze significantly less in Context B than in Context A. By Day 10, mice in both control and experimental groups discriminated between the two contexts. However, mice administered Bay 55-9837 exhibited a greater difference in freezing between Context A and Context B when compared to saline. (F-G) Both saline and Bay 55-9837-administered male mice exhibited contextual fear discrimination. Saline mice began exhibiting fear discrimination on Day 5, while Bay 55-9837-administered mice began fear discriminating on Day 7. (H) There was no significant difference in discrimination index between saline and Bay 55-9837-administered male mice. (I) By Day 10, freezing in both contexts were comparable between both groups. In male mice, freezing in Context B was significantly lower than freezing in Context B in both saline and Bay 55-9837 groups. Error bars represent±SEM. * p<0.05, ** P<0.01, *** p<0.001, **** p<0.0001. Hrs, hours.

FIG. 7 shows that fear discrimination learning increases VPAC2 receptor expression. (A) Experimental protocol. Mice were administered a contextual fear discrimination assay. Mice were sacrificed after Context B exposure either on Day 4 (high fear generalization) or on Day 10 (low fear generalization). Following euthanasia, brain-wide immunolabeling and imaging was used to assay VPAC2 expression throughout the brain. (B-C) Mice sacrificed on Day 4 exhibited high levels of fear generalization. Freezing was not significantly different between Contexts A and B. (D-E) Mice sacrificed on Day 11 exhibited low levels of fear generalization. By Day 11, freezing in Context B was significantly reduced when compared to freezing in Context A. (F) Experimental protocol for brain-wide VPAC2 immunolabeling. Following Context B exposure, mice were sacrificed, and brains were harvested. Brains were serially sectioned, stained for VPAC2 expression using immunohistochemistry, and imaged. An automated cell counting pipeline was used to segment the cells, register brains to an atlas, and integrate cell counts for further analysis. (G) Sample VPAC2 staining (left). Brains were automatically registered to a brain atlas (middle), and cell counts were integrated to obtain cell counts per brain region (right). (H-I) Mice in the low fear generalization group exhibited significantly higher VPAC2 expression in the agranular insula area and CA3 of the hippocampus. Error bars represent±SEM. * p<0.05, ** P<0.01, *** p<0.001, **** p<0.0001. Hrs, hours; CFD, contextual fear discrimination; no., number; RSP, retrosplenial area; ACA, anterior cingulate area; PERI, perirhinal arca; TEa, temporal association areas; AI, agranular insula arca; ECT, ectorhinal area; GU, gustatory arcas; PIR, piriform arca; PAA, piriform-amygdalar arca; COA, cortical amygdalar arca; EP, endopiriform nucleus; CLA, claustrum; BLA, basolateral amygdalar nucleus; BMA, basomedial amygdalar nucleus; LA, lateral amygdalar nucleus; ME, median eminence; AUDd, dorsal auditory arca; AUDp, primary auditory area; AUDv, ventral auditory area; MOs, secondary motor arca; MOp, primary motor arca; SSs, supplemental somatosensory area; SSp-tr, primary somatosensory arca, trunk; SSp-m, primary somatosensory arca, mouth; SSp-bfd, primary somatosensory arca, barrel field; DG, dentate gyrus; CA3, field CA3; CA2, field CA2; CA1, field CA1.

FIG. 8 shows that Bay 55-9837 reduces neural activity in vCA3 during CFD learning in female mice. (A) Experimental paradigm. Female mice were injected with GCaMP6f17 virus and a GRIN lens in vCA3 of the hippocampus, then base-plated following viral expression. Mice were then injected with saline or Bay 55-9837 (2.5 mg/kg) one week prior to administration of a CFD paradigm. Calcium imaging was performed during injection as well as Days 1, 2, 5, and 10 of the behavioral paradigm. (B-E) As previously demonstrated, Bay 55-9837 facilitated and enhanced CFD in female mice when compared to saline controls. (F-H) Neural activity was not significantly altered during injection of Bay 55-9837. (I) However, upon the first Context A exposure, Bay 55-9837 significantly reduced neural activity in vCA3. (J) Directly after Context A learning, Bay 55-9837 significantly increased neural activity compared to saline controls. On Day 2 of CFD, Bay 55-9837 did not significantly alter neural activity in (K-L) Context A, but did reduce activity in (M-O) Context B, suggesting that Bay 55-9837 reduces neural activity during initial learning exposures in CFD. On (P-T) Day 5 and (U-Y) Day 10 of CFD, Bay 55-9837 did not significantly alter neural activity compared to controls, suggesting that this suppression of activity is unique to the initial learning of the CFD contexts. Error bars indicate+SEM. * p<0.05, ** p<0.01, *** p<0.001, **** p<0.0001. Ca²⁺, calcium; hrs, hours; no., number; Hz, hertz; Sal, saline; Bay, Bay 55-9837.

FIG. 9 shows that VPAC2 receptor inhibition and activation occludes and mimics the prophylactic effects of (R,S)-ketamine in female mice. (A) Experimental protocol. Female 129S6/SvEv mice were administered an intracerebroventricular infusion of artificial cerebrospinal fluid, PACAP (6-38) (VPAC2 antagonist), or Bay 55-9837. Five minutes after, mice were given an IP injection of saline or (R,S)-ketamine (10 mg/kg). One week later, mice were administered 3-shock CFC training. Five days subsequently, mice were tested in CFC re-exposure and the FST. (B) During CFC training, there was no significant difference in freezing across all groups. (C-D) Upon CFC re-exposure, there was a significant group x drug interaction. Freezing was not significantly different between saline- and (R,S)-ketamine-administered mice in both the Vehicle and PACAP(6-38) groups. Bay 55-9837/saline-administered mice exhibited significantly lower freezing when compared to both vehicle/saline and Bay 55-9837/(R,S)-ketamine groups. (E) Immobility time on FST Day 1 was comparable across all groups. (F-G) On FST Day 2, Vehicle/(R,S)-ketamine-administered mice had significantly lower immobility when compared with Vehicle/saline-administered mice. In the PACAP (6-38) group, the prophylactic effect of (R,S)-ketamine was blocked. Bay 55-9837/saline-administered mice had significantly lower immobility when compared to the Vehicle/saline group. However, in Bay 55-9837/(R,S)-ketamine-administered mice, immobility time was comparable to Vehicle/saline controls. Error bars represent±SEM. * p<0.05, ** P<0.01, *** p<0.001, **** p<0.0001. ICV, intracerebroventricular; IP, intraperitoneal; CFC, contextual fear conditioning; FST, forced swim test; mg, milligram; kg, kilogram; min, minute; Sal, saline; K, (R,S)-ketamine; sec, second.

FIG. 10 shows that Bay 55-9837 does not alter behavioral despair in non-stressed mice. (A) Experimental protocol. Female and male 129S6/SvEv mice were given a single injection of saline, (R,S)-ketamine (10 mg/kg in females, 30 mg/kg in males), or Bay 55-9837 (2.5 mg/kg in females, 10 mg/kg in males) one hour prior to FST Day 1. FST Day 2 was administered 1 day later. (B-E) In female mice, immobility time was comparable between all groups on both FST Day 1 and FST Day 2. (F-I) Similarly, in male mice, drug administration did not significantly alter immobility time on either day of the FST. Error bars represent±SEM. * p<0.05, ** P<0.01, *** p<0.001, **** p<0.0001. FST, forced swim test; sec, second; min, minute; Sal, saline; K, (R,S)-ketamine; Bay, Bay 55-9837; mg, milligram; kg, kilogram.

FIG. 11 shows that Bay 55-9837 suppresses hyponcophagia in naïve female mice. (A) Experimental protocol. Female 129S6/SvEv mice were administered a single injection of saline or Bay 55-9837 (2.5 mg/kg) one hour prior to the OF. On subsequent days, mice were administered the MB, EPM, and NSF assays. (B-D) Locomotion and time spent in the center of the OF were comparable between both groups. (E-H) Similarly, exploratory behavior in the EPM was not significantly altered by Bay 55-9837 administration. (I) Bay 55-9837-administered mice buried a comparable amount of marbles as saline controls. (J-K) Bay 55-9837 significantly reduced latency to feed in the OF during the NSF. Error bars represent±SEM. * p<0.05, ** P<0.01, *** p<0.001, **** p<0.0001. Sal, saline; OF, open field; MB, marble burying; EPM, elevated plus maze; NSF, novelty-suppressed feeding; cm, centimeter; min, minute; Bay, Bay 55-9837; mg, milligram; kg, kilogram; sec, second; no., number.

FIG. 12 shows that Bay 55-9837 reduces perseverative behavior and suppresses hyponcophagia in male mice. (A) Experimental protocol. Male 129S6/SvEv mice were administered the same behavioral paradigm as in FIG. 11A. (B-D) Distance travelled and time spent in the center of the OF was comparable between both groups. (E) There was a trending, but not significant, increase in time spent in the open arms and center of the EPM. (F) However, entries into the open arms and center were not significantly different between both groups. (G) Similarly, there was a trending, but not significant, decrease in time spent in the closed arms. (H) Entries into the closed arms was not significantly affected by drug administration. (I) Bay 55-9837-administered mice buried significantly less marbles when compared to saline controls. (J-K) Bay 55-9837-administered mice also exhibited a significantly reduced latency to feed in the OF during the NSF. Error bars represent±SEM. * p<0.05, ** P<0.01, *** p<0.001, **** p<0.0001. Sal, saline; OF, open field; MB, marble burying; EPM, elevated plus maze; NSF, novelty-suppressed feeding; cm, centimeter; min, minute; Bay, Bay 55-9837; mg, milligram; kg, kilogram; sec, second; no., number.

FIG. 13 shows that fear discrimination learning does not alter VPAC2 receptor expression in the thalamus, cerebral nuclei, or hypothalamus. VPAC2 receptor expression was comparable across all regions in the brain in the (A) thalamus, (B) cerebral nuclei, and (C) hypothalamus. Error bars represent±SEM. * p<0.05, ** P<0.01, *** p<0.001, **** p<0.0001. GENv, geniculate group, ventral thalamus; RT, reticular nucleus of the thalamus; RH, rhomboid nucleus; CL, central lateral nucleus of the thalamus; PCN, paracentral nucleus; CM, central medial nucleus of the thalamus; MD, mediodorsal nucleus of the thalamus; IMD, intermediodorsal nucleus of the thalamus; SMT, submedial nucleus of the thalamus; PO, posterior complex of the thalamus; LP, lateral posterior nucleus of the thalamus; PVT, paraventricular nucleus of the thalamus; RE, nucleus of reuniens; LH, lateral habenula; MH, medial habenula; LD, lateral dorsal nucleus of the thalamus; SPF, subparafascicular nucleus; GENd, geniculate group, dorsal thalamus; VAL, ventral anterior-lateral complex; VM, ventral medial nucleus of the thalamus; VP, ventral posterior complex; VPM, ventral posterolateral nucleus; SI, substantia innominate; NDB, diagonal band nucleus; GPc, globus pallidus, external segment; ACB, nucleus accumbens; CP, caudoputamen; LS, lateral septal nucleus; MEA, medial amygdalar nucleus; IA, intercalated amygdalar nucleus; CEA, central amygdalar nucleus; VPL, ventral posterolateral nucleus; LHA, lateral hypothalamic area; TU, tuberal nucleus; ZI, zona incerta; STN, subthalamic nucleus; PSTN, parasubthalamic nucleus; ARH, arcuate hypothalamic nucleus; VMH, ventromedial hypothalamic nucleus; PH, posterior hypothalamic nucleus; DMH, dorsomedial nucleus of the hypothalamus.

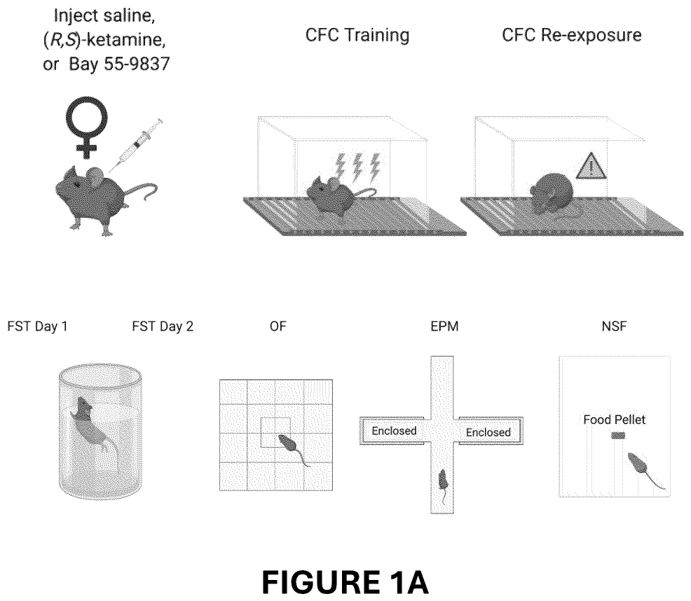

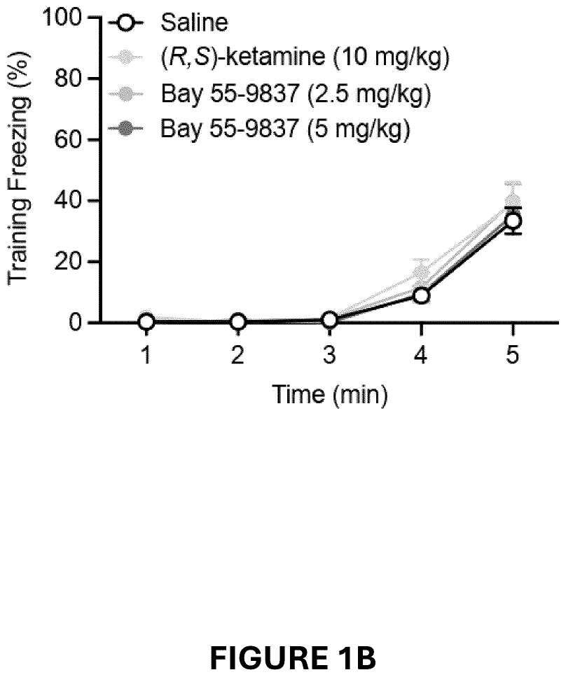

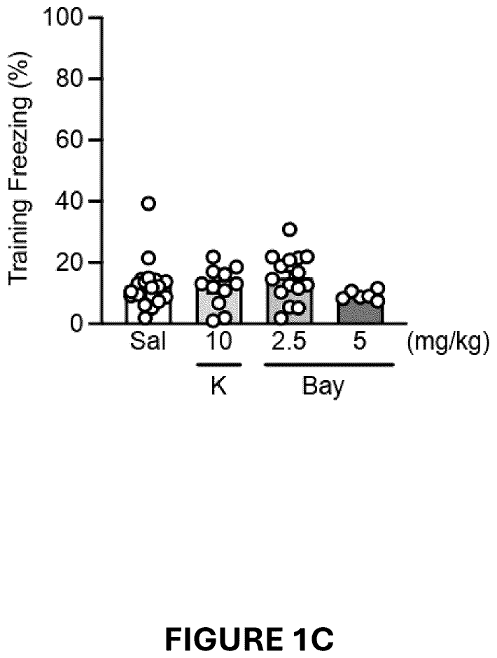

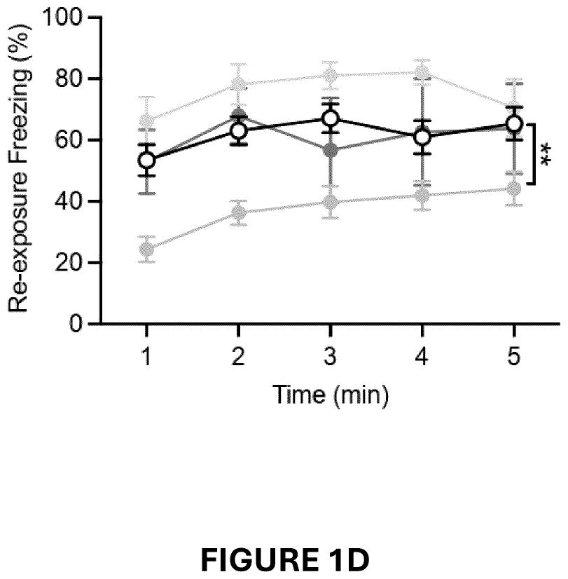

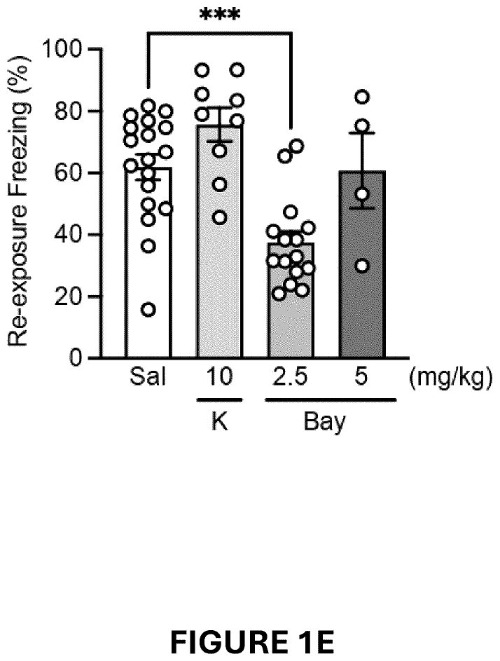

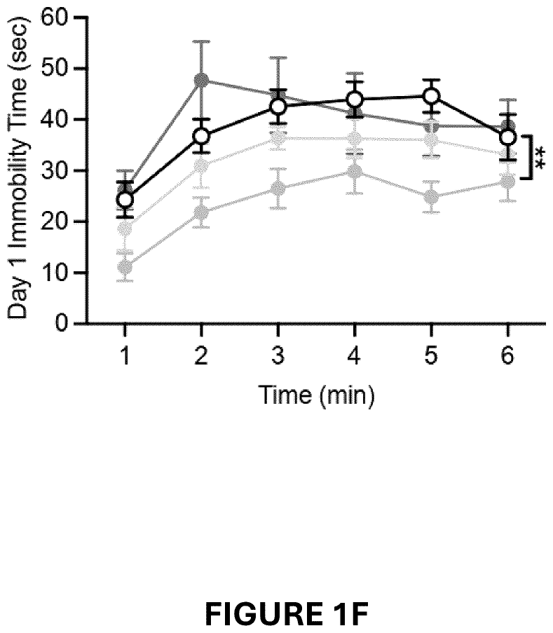

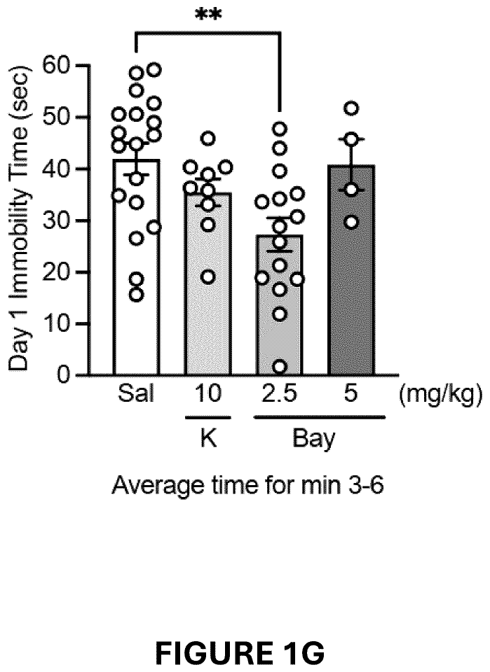

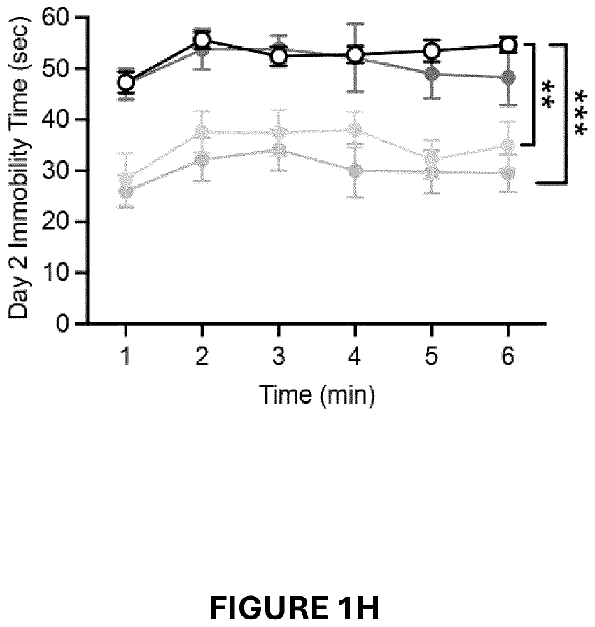

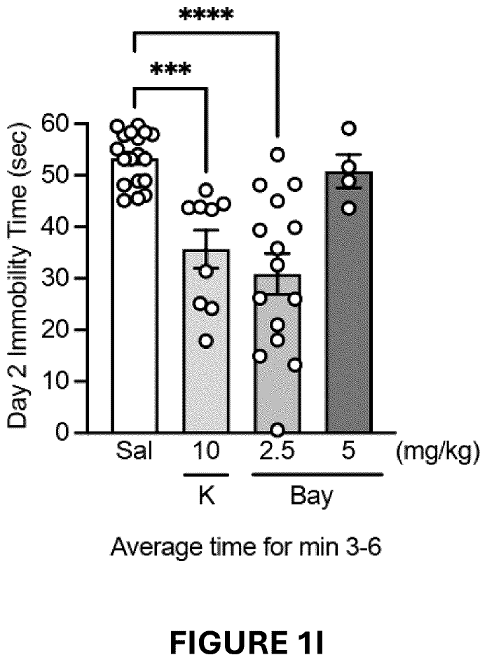

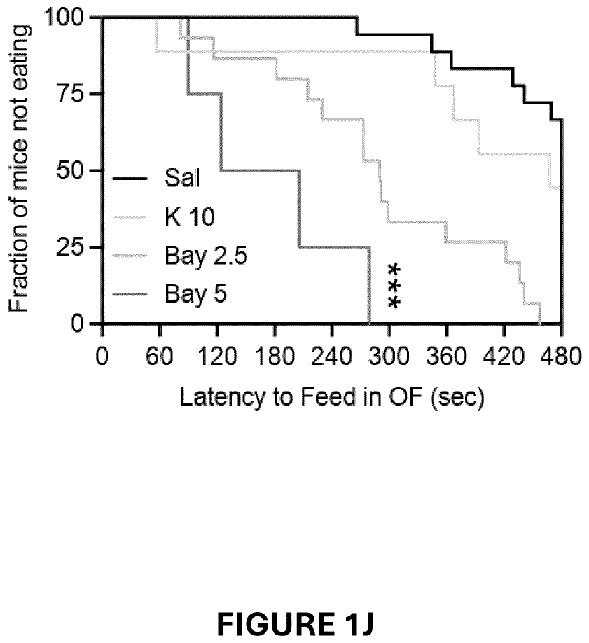

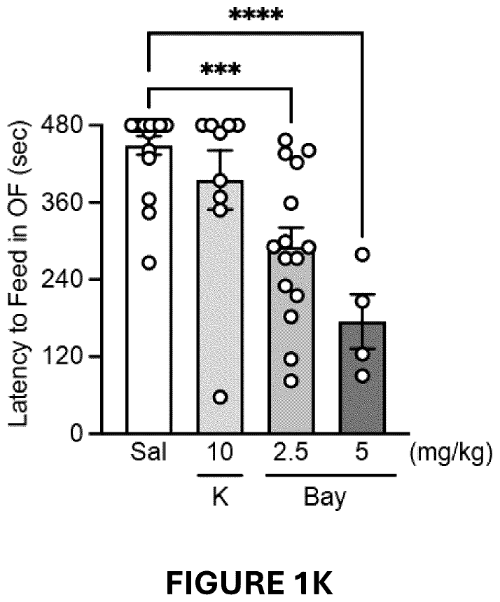

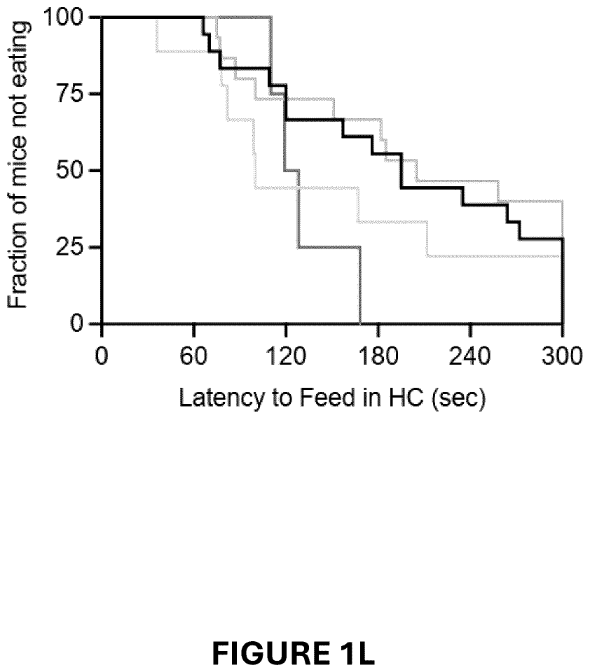

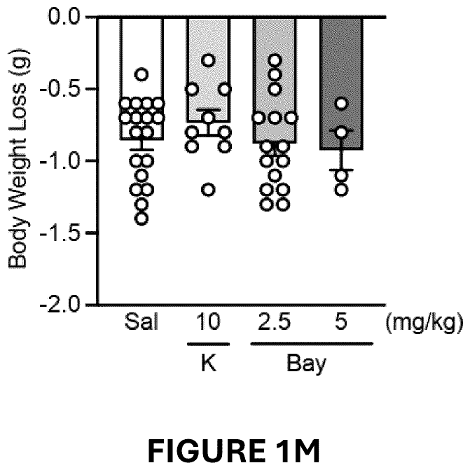

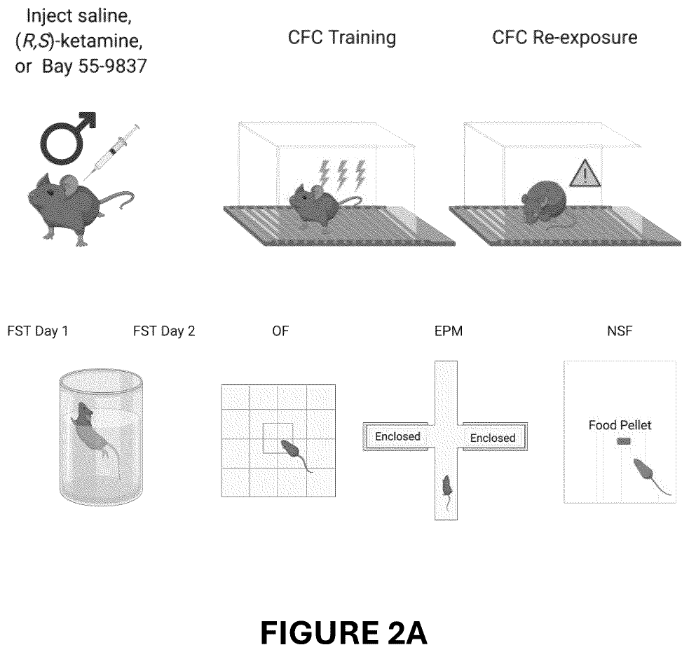

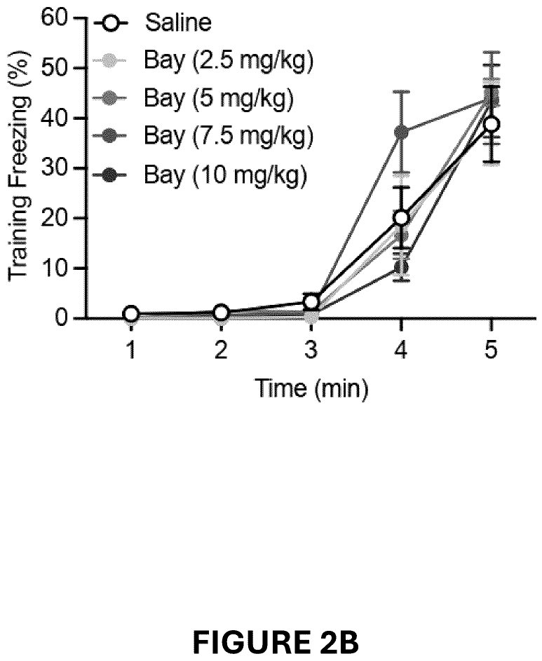

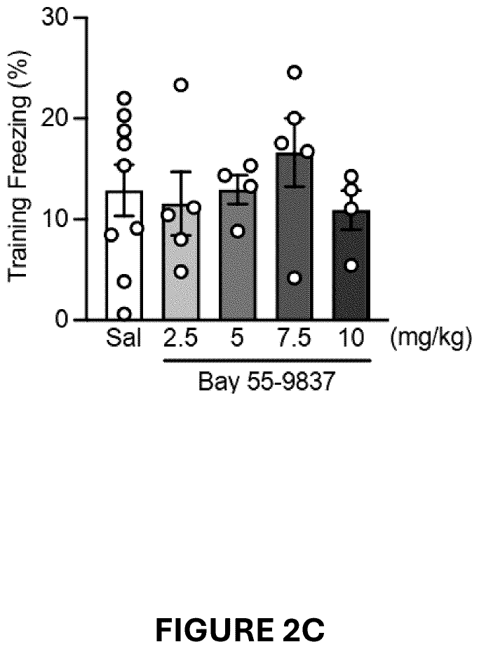

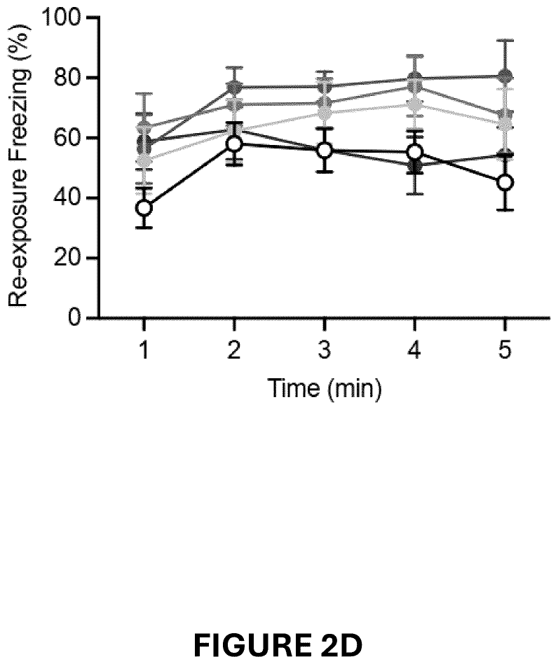

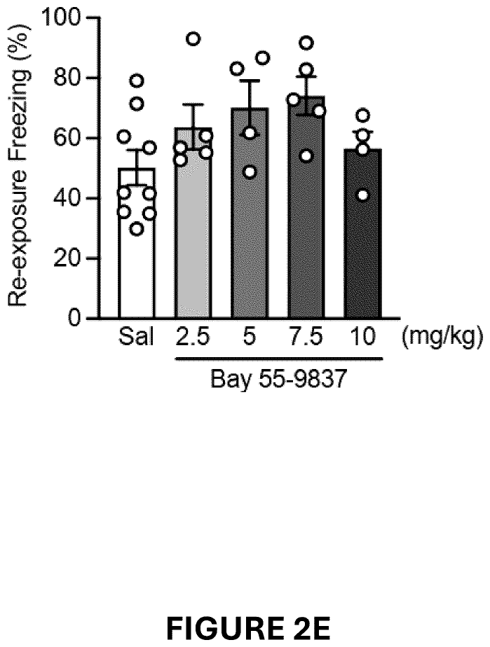

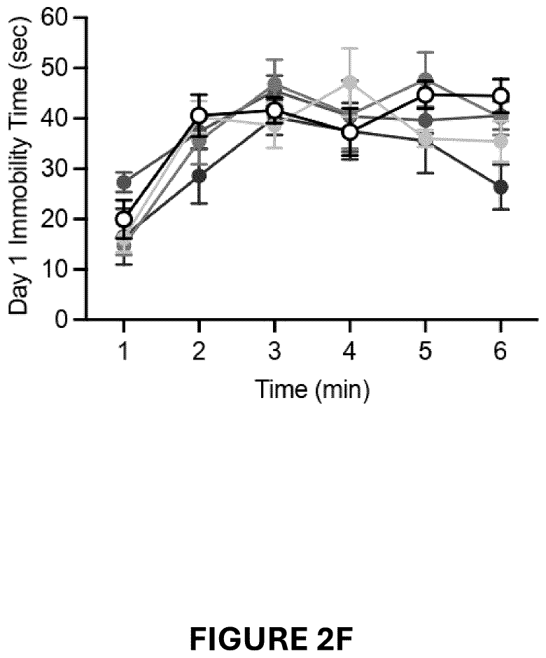

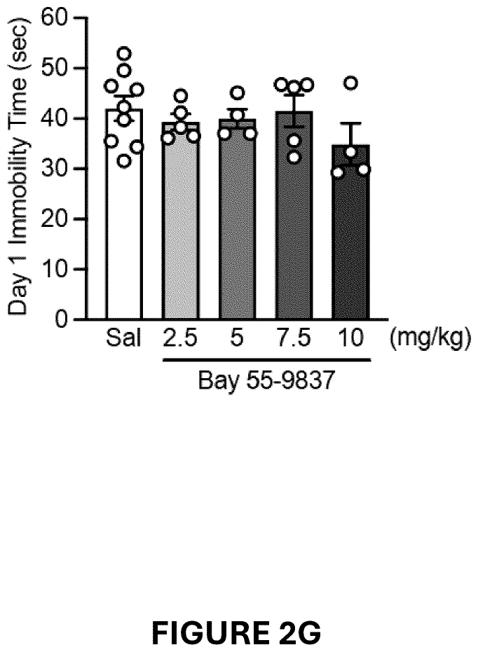

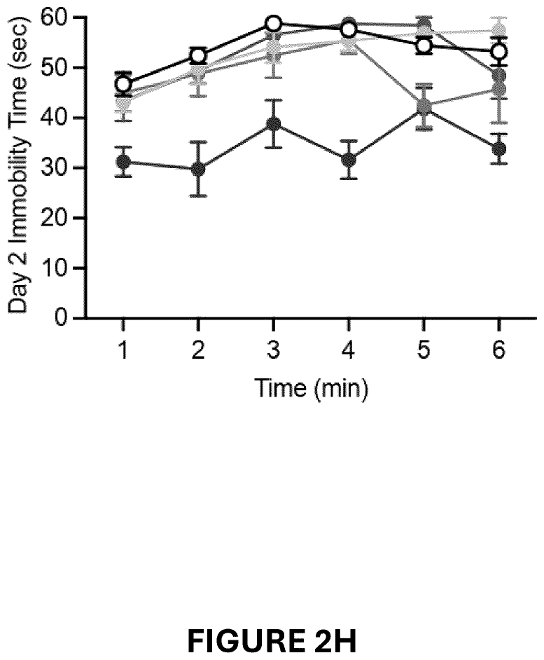

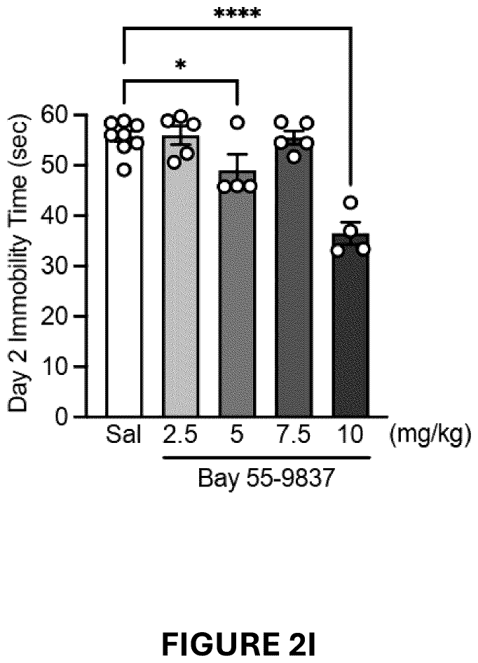

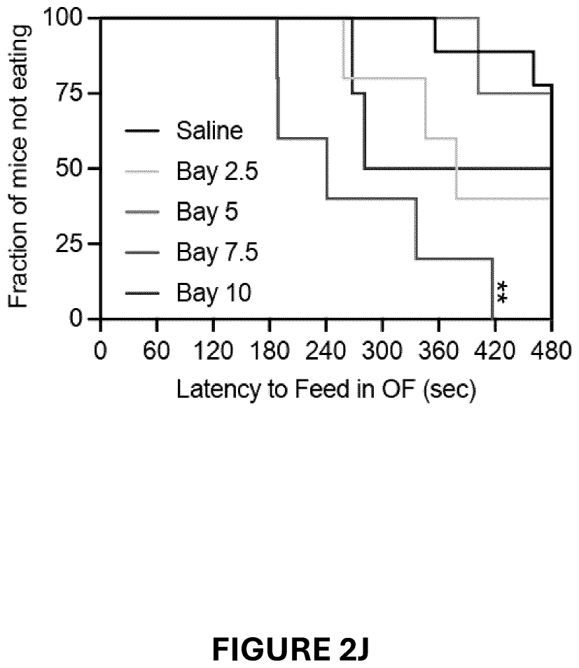

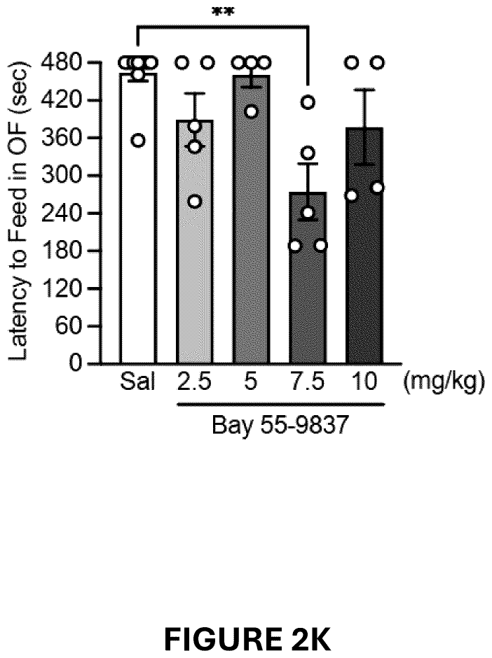

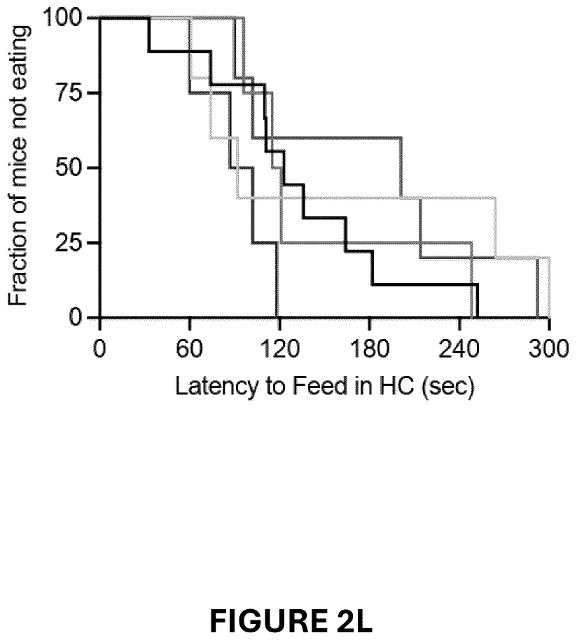

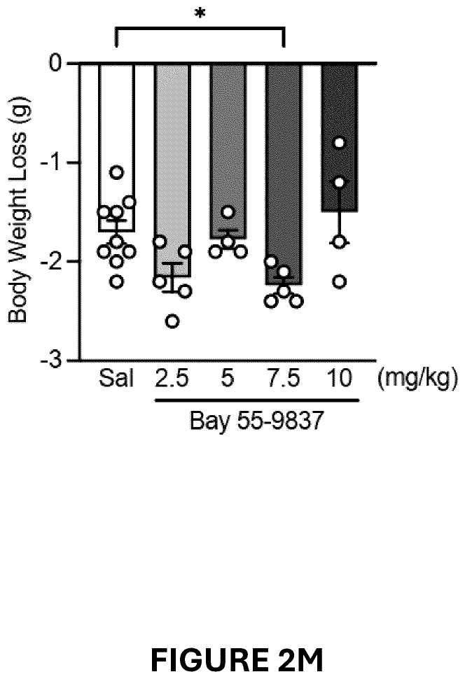

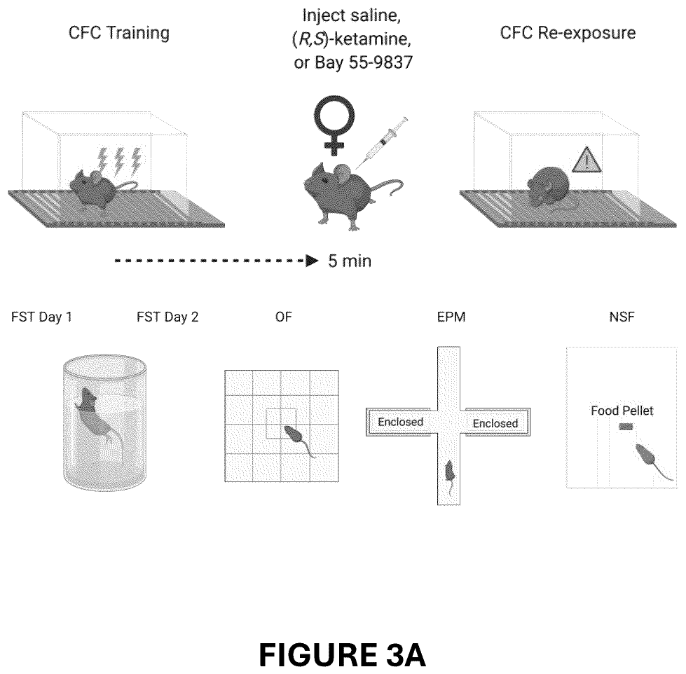

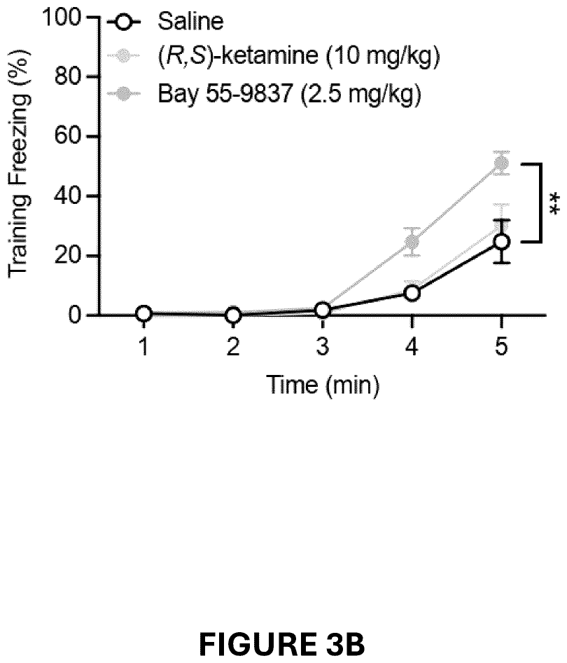

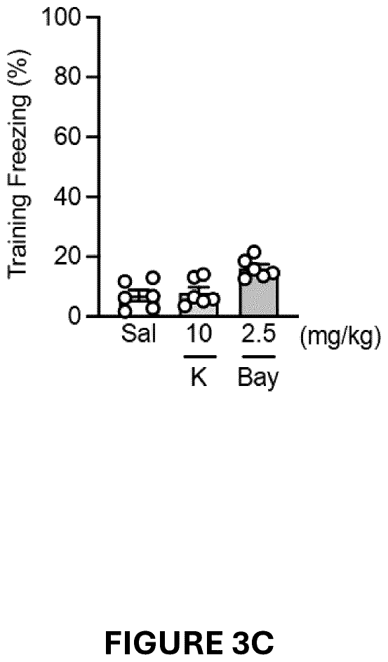

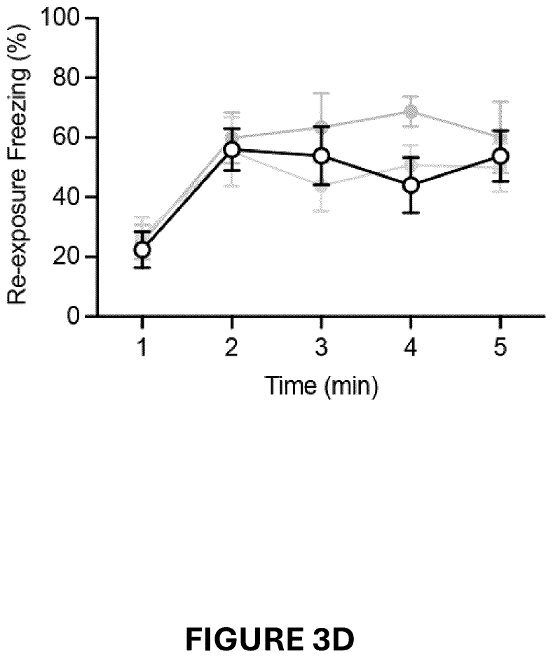

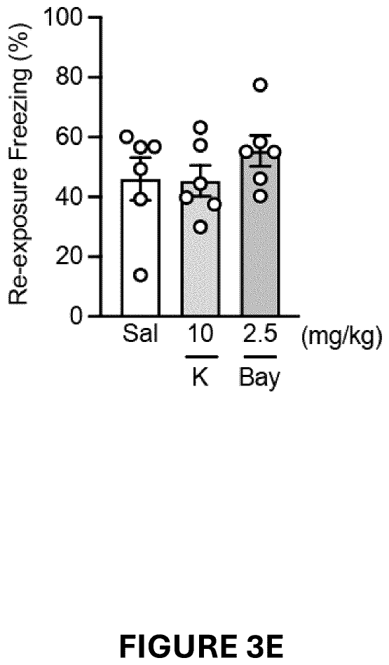

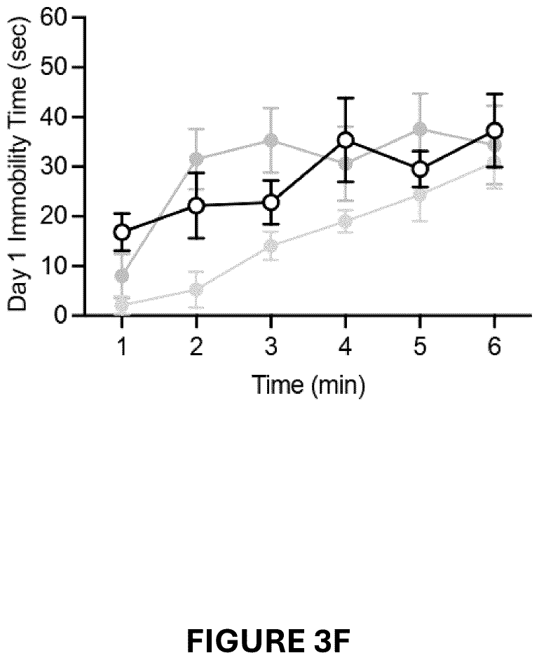

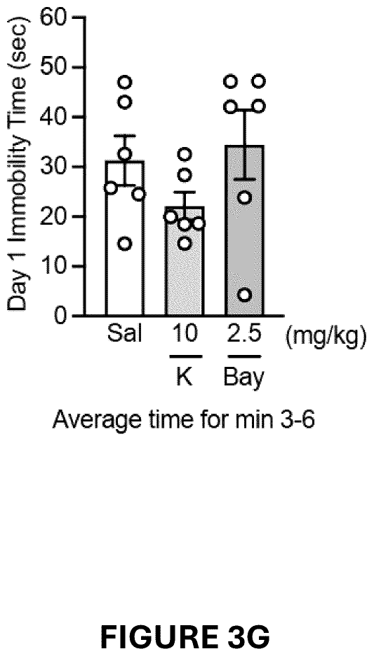

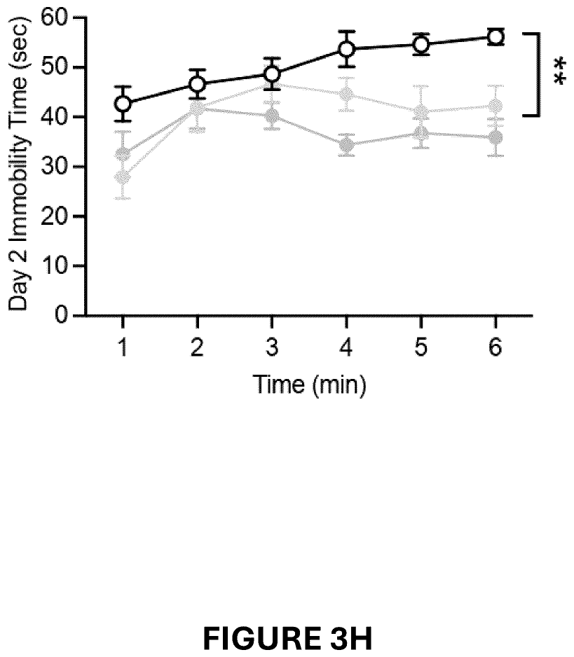

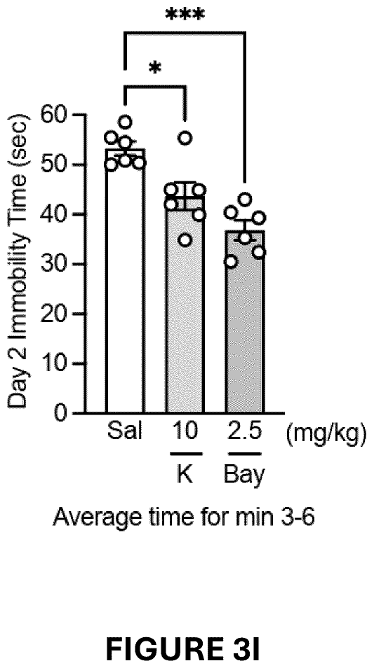

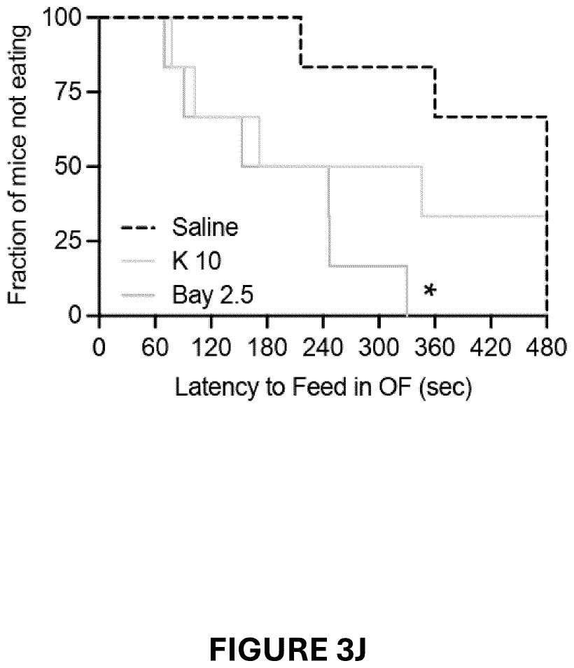

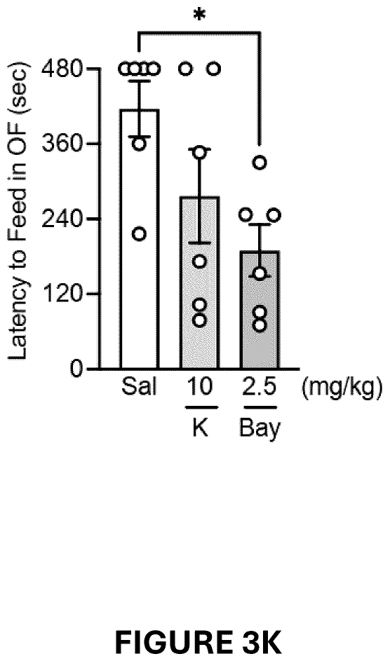

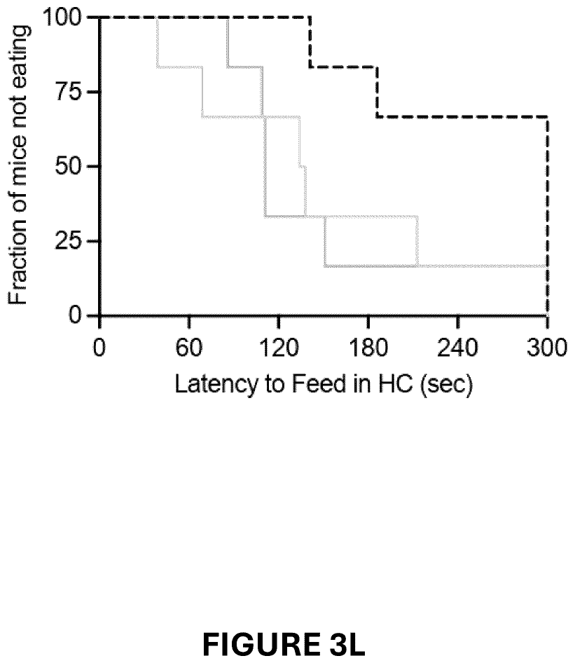

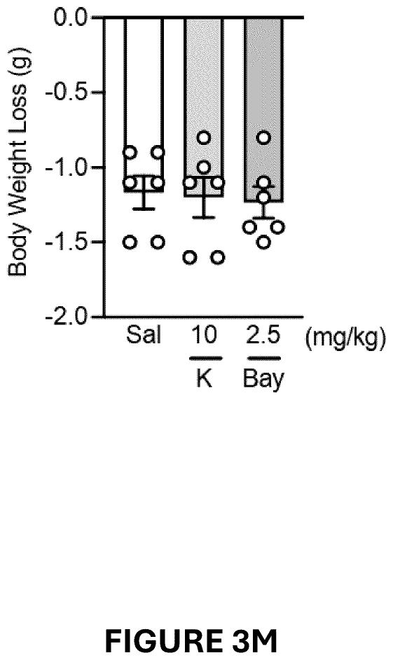

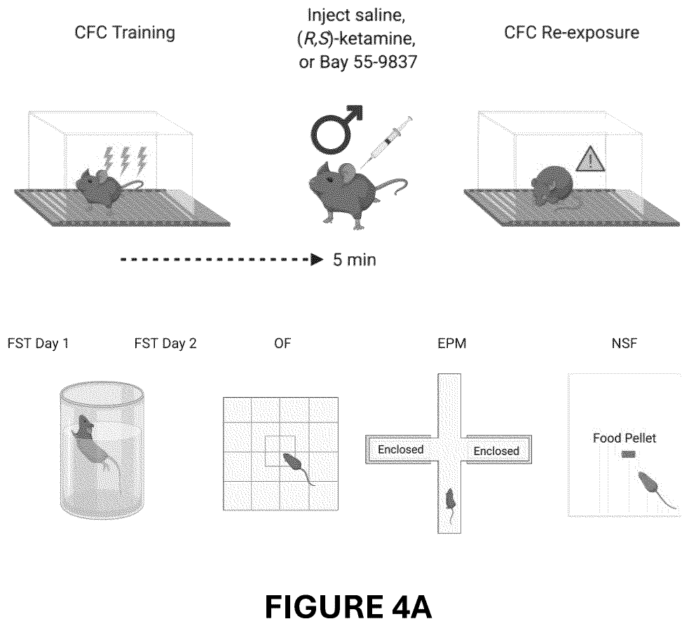

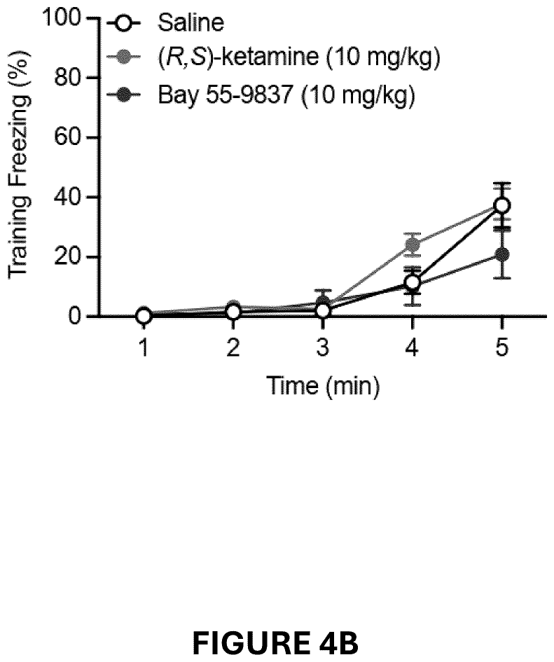

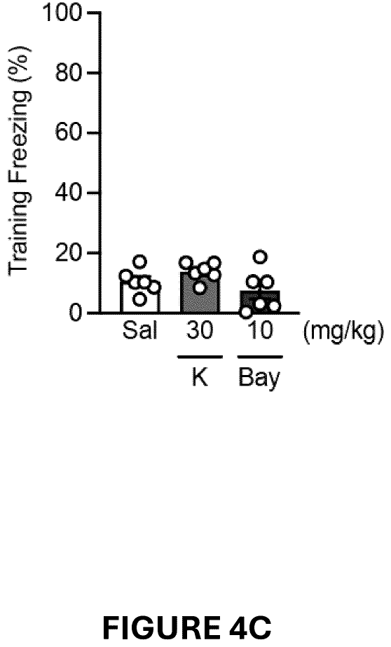

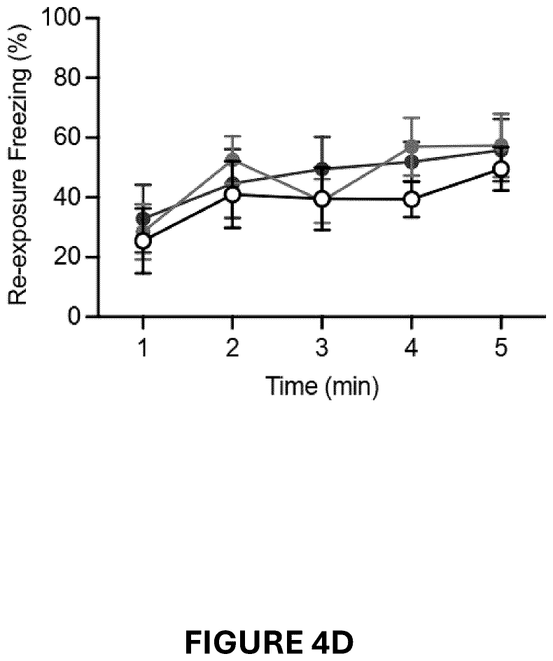

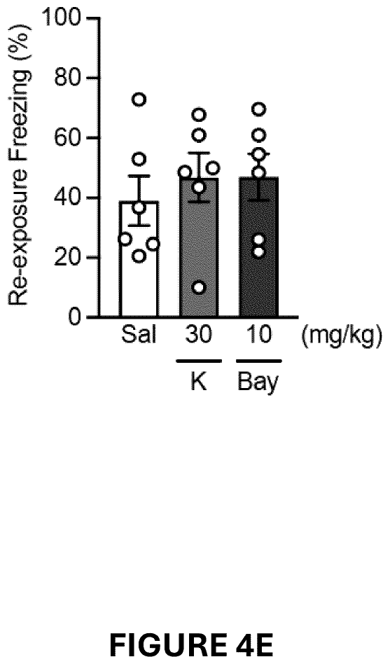

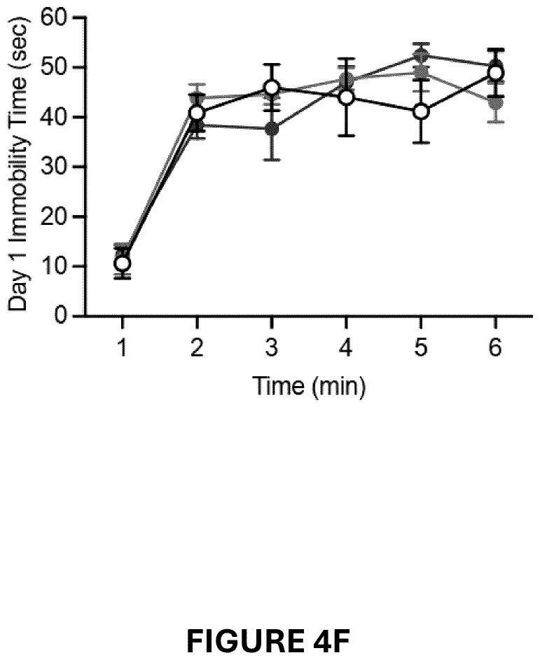

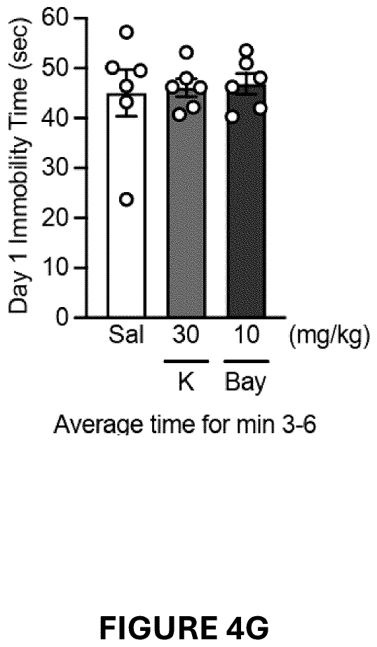

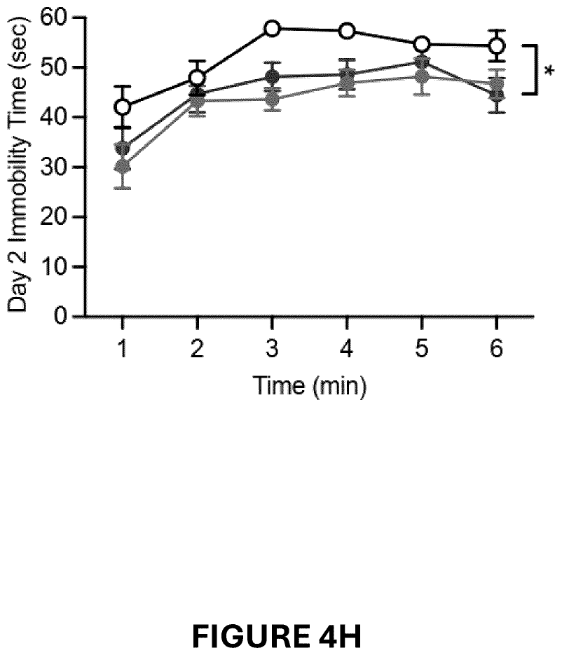

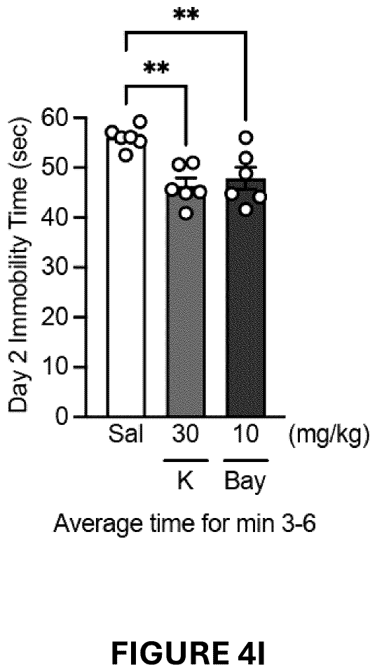

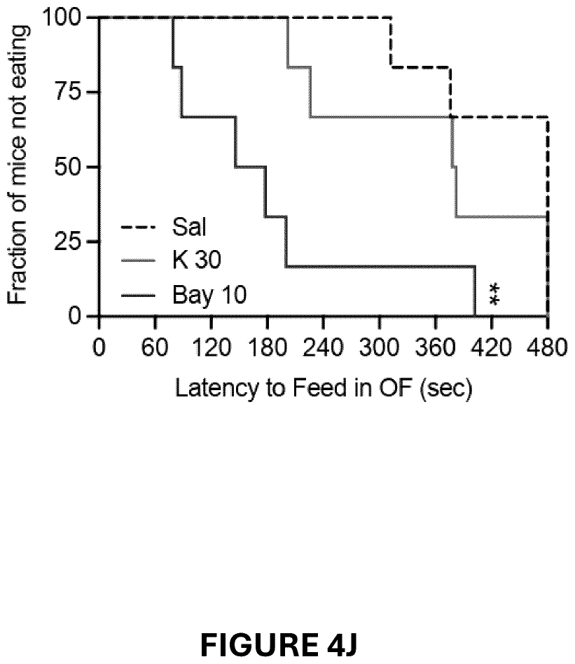

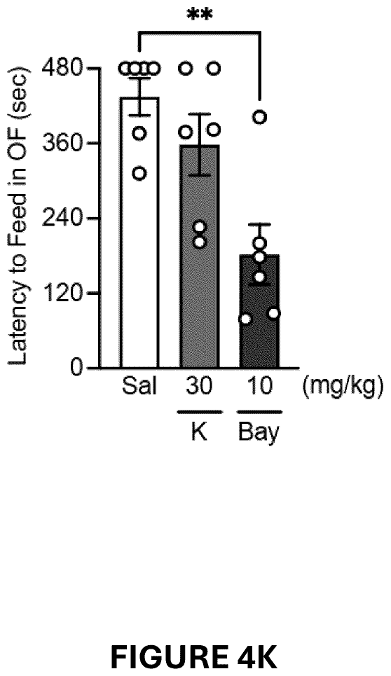

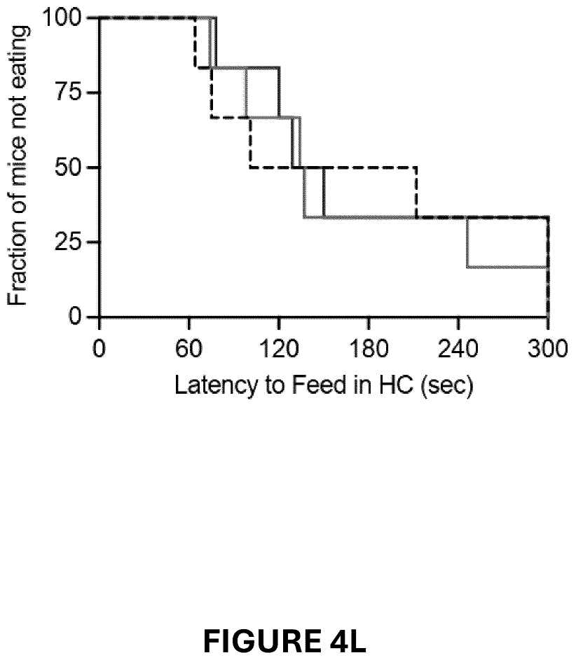

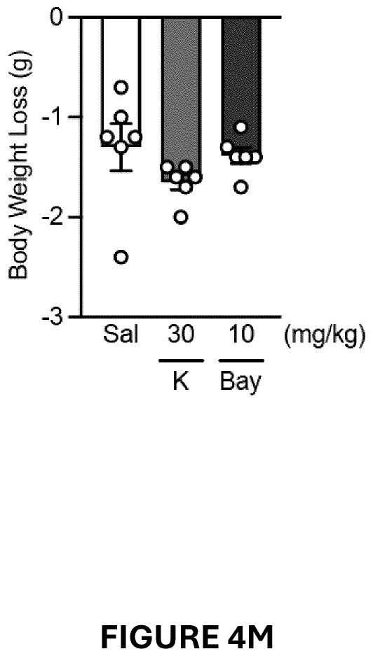

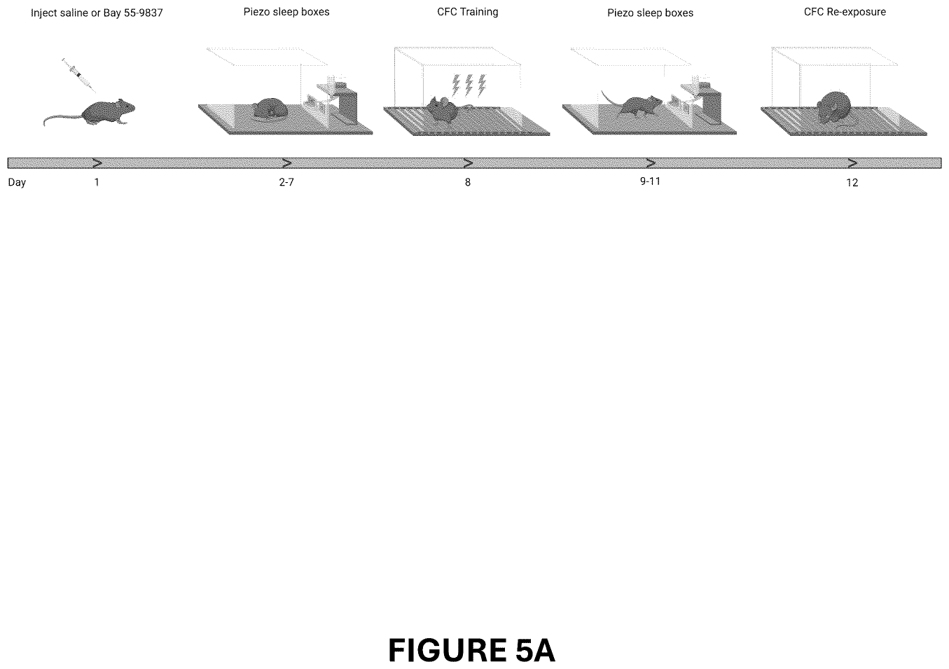

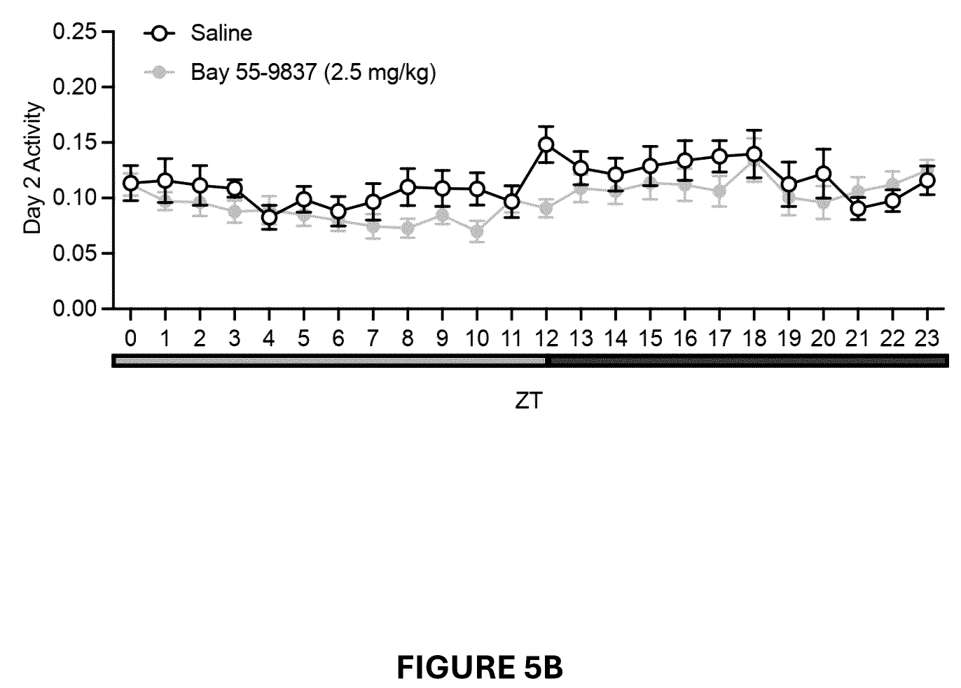

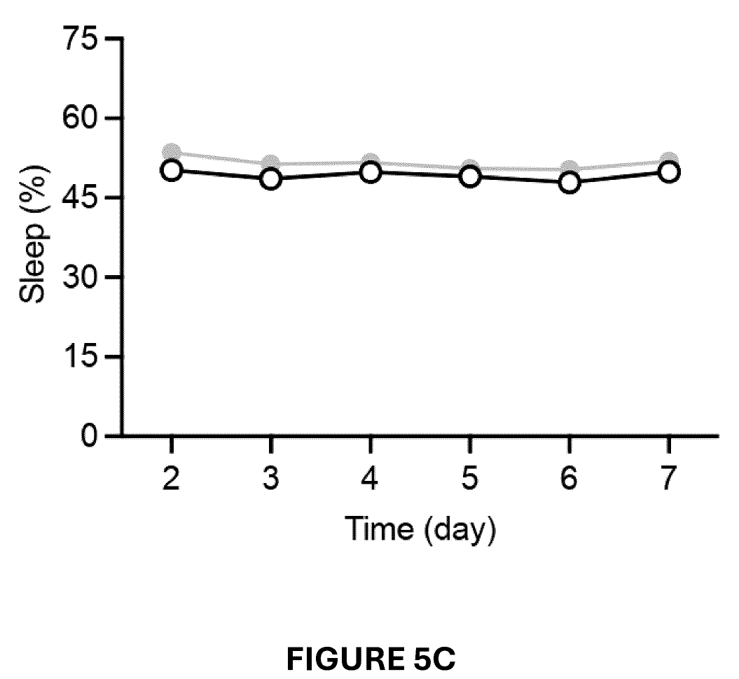

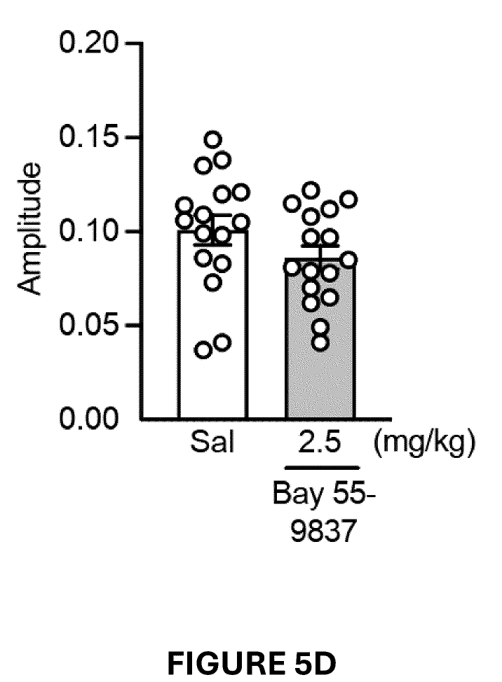

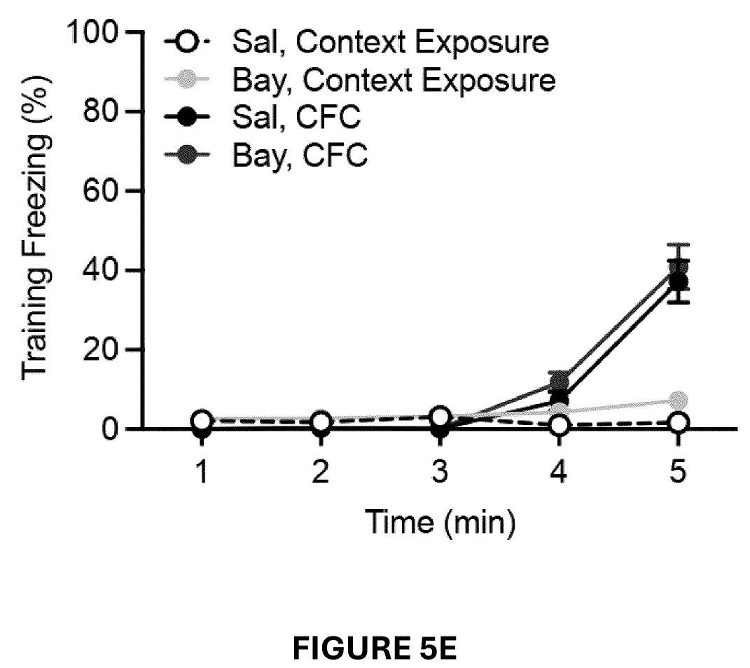

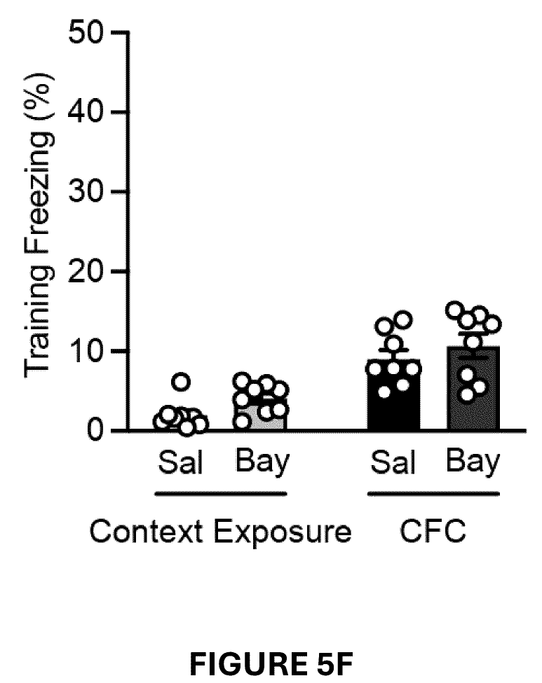

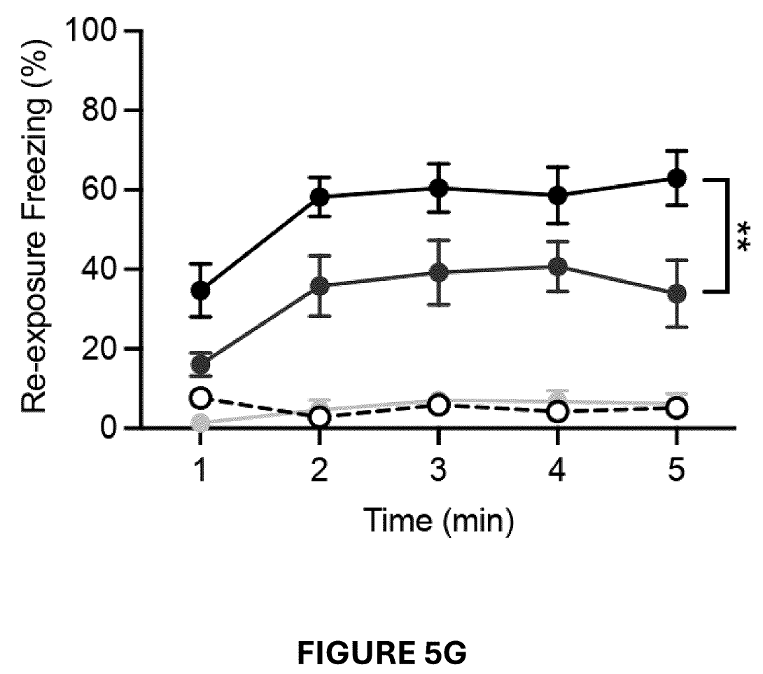

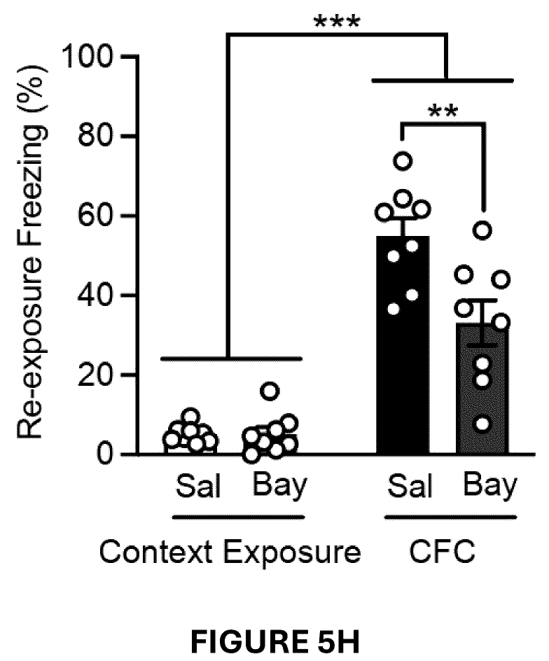

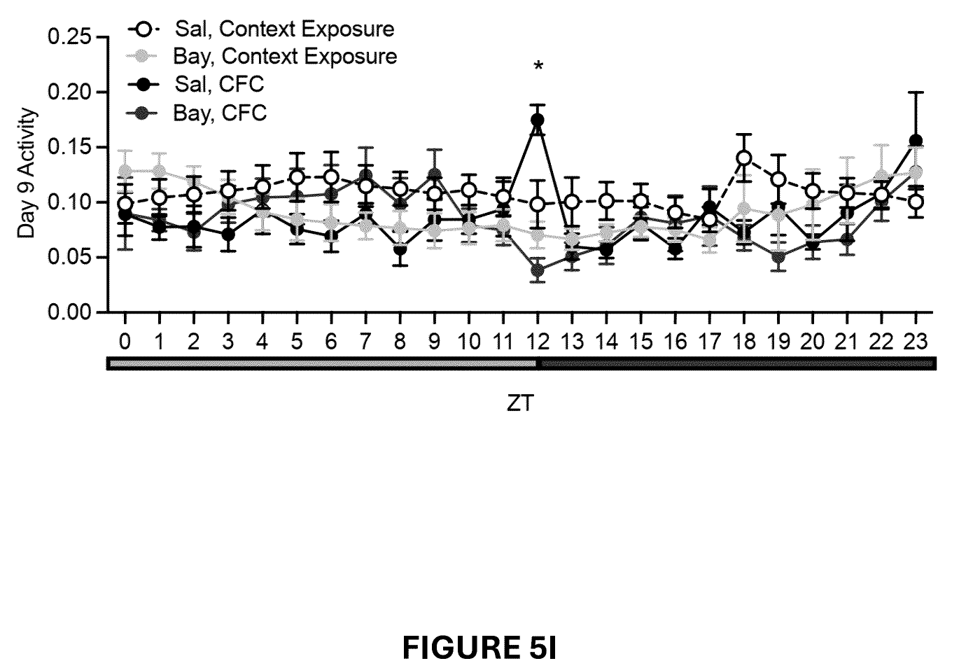

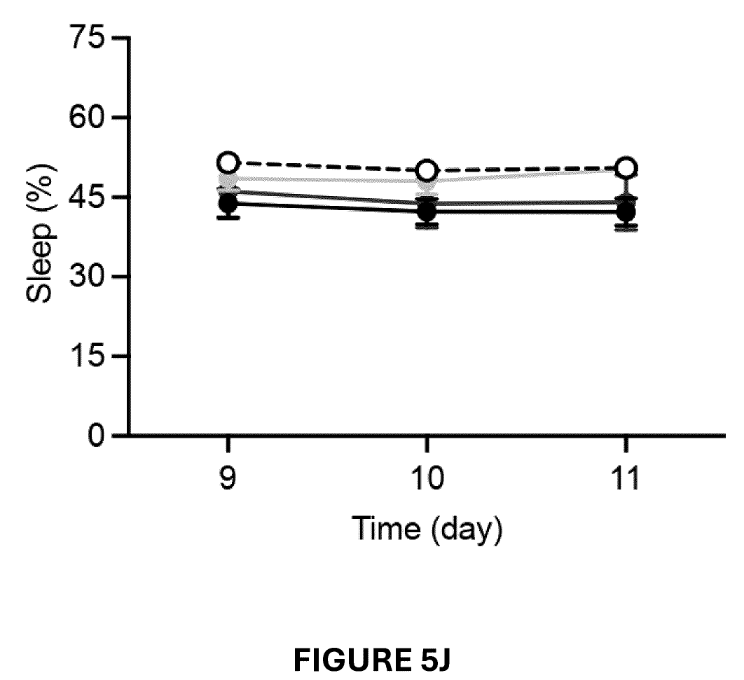

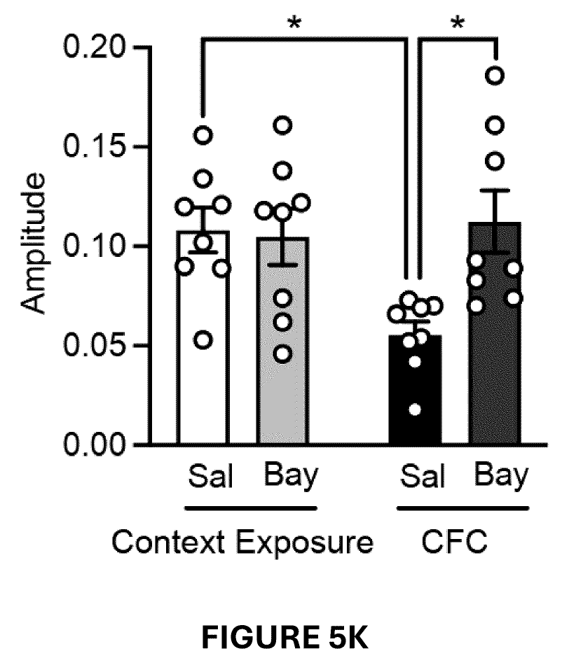

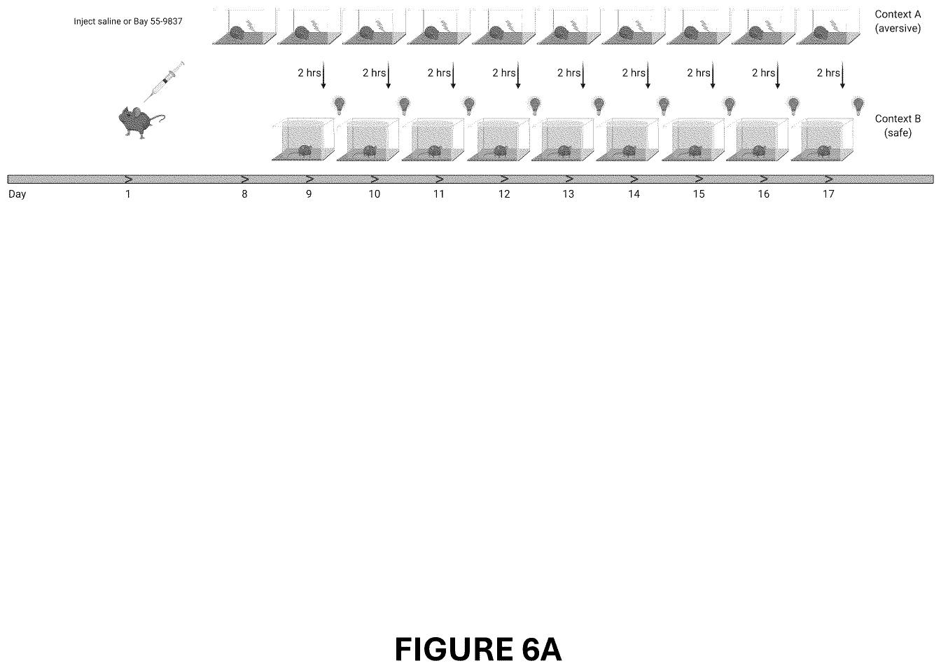

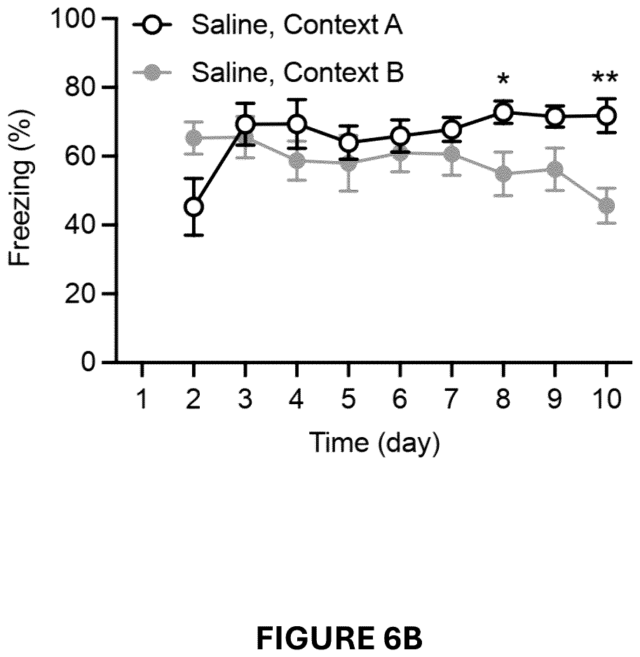

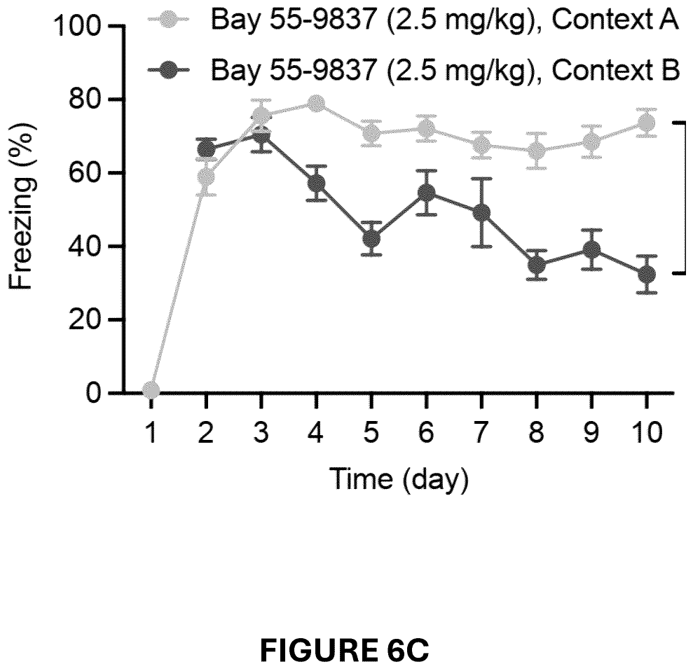

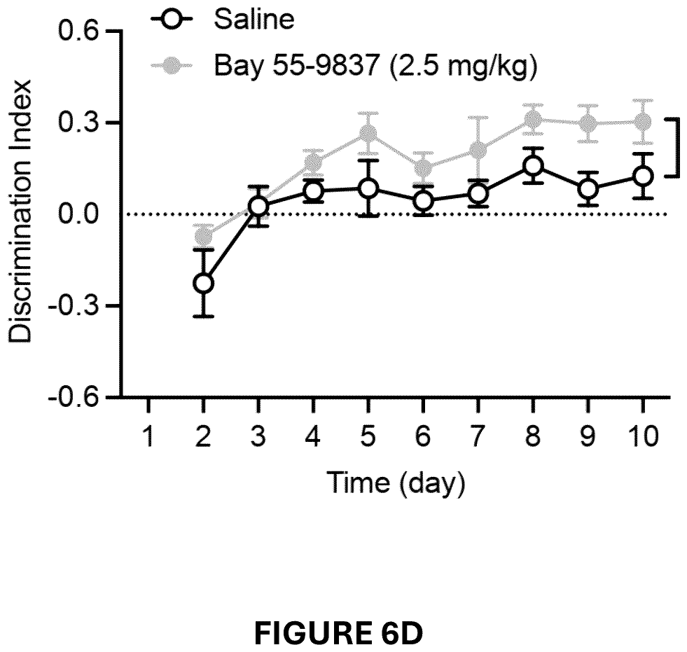

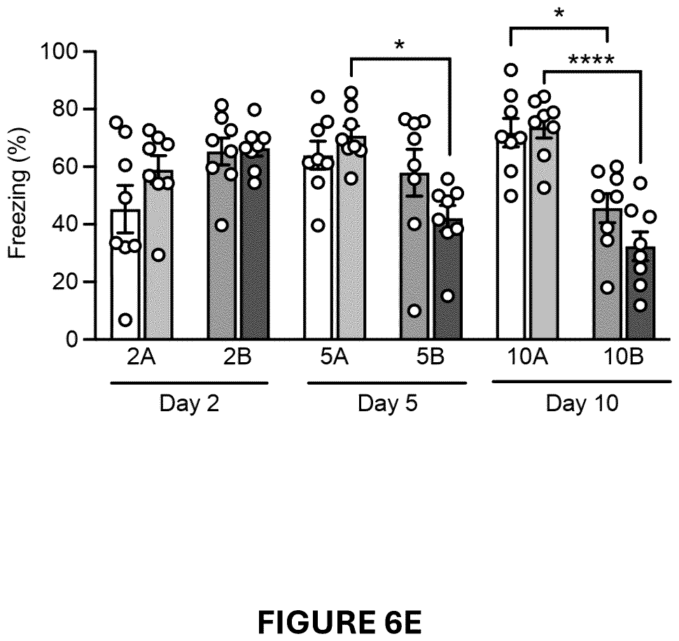

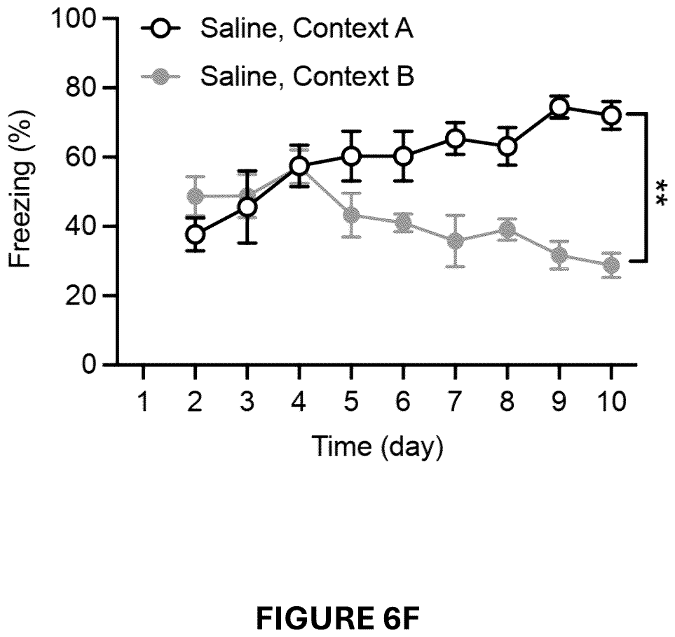

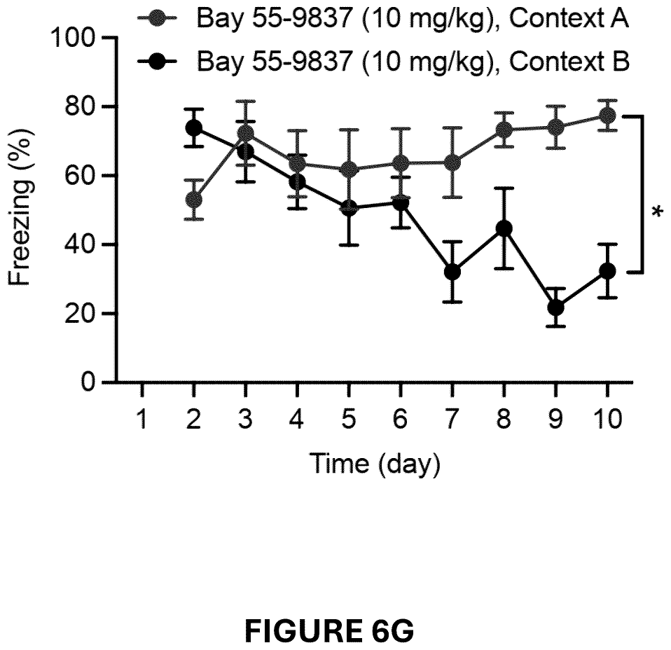

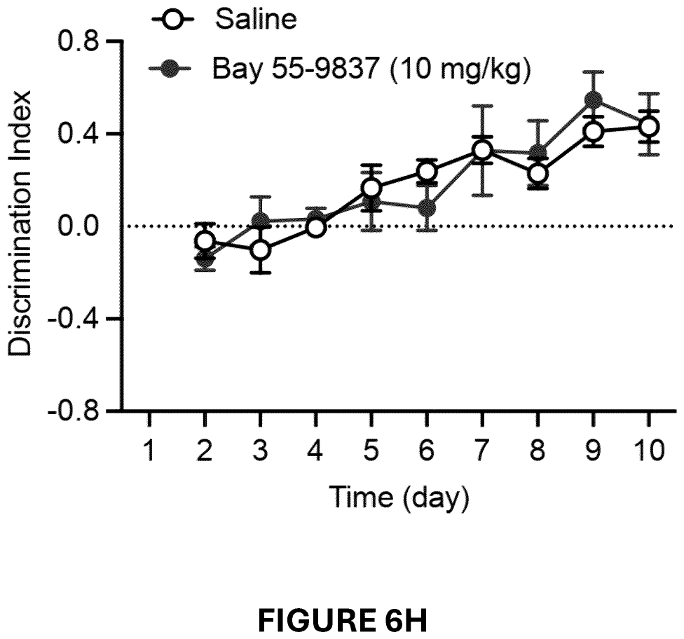

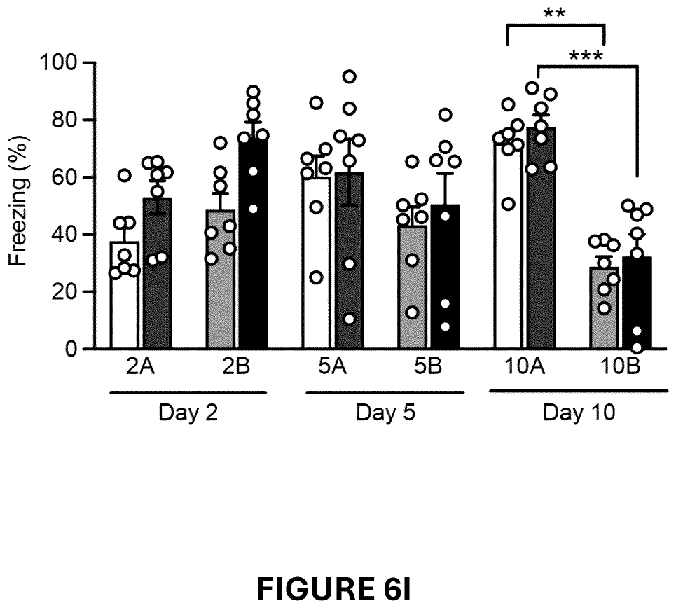

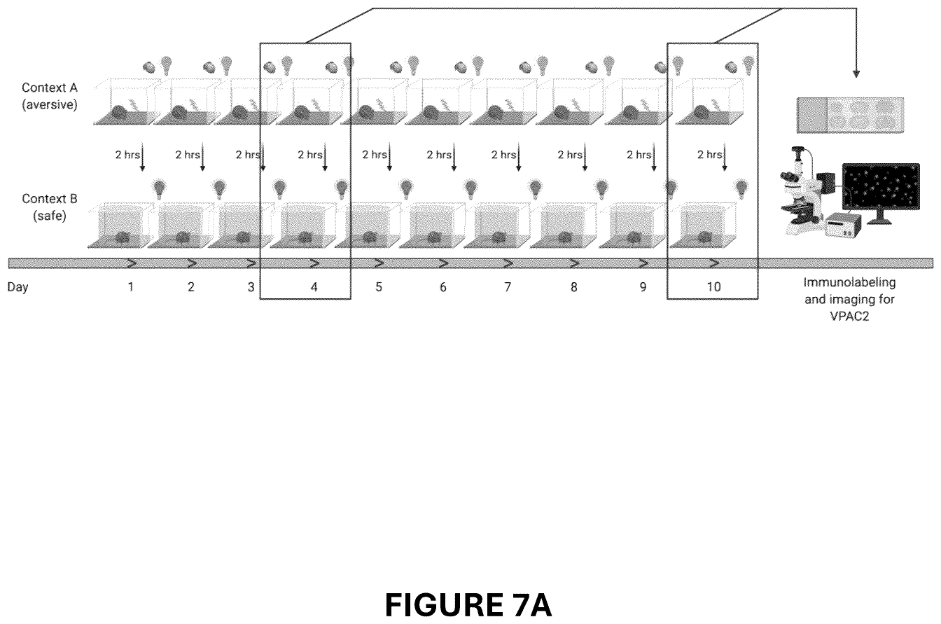

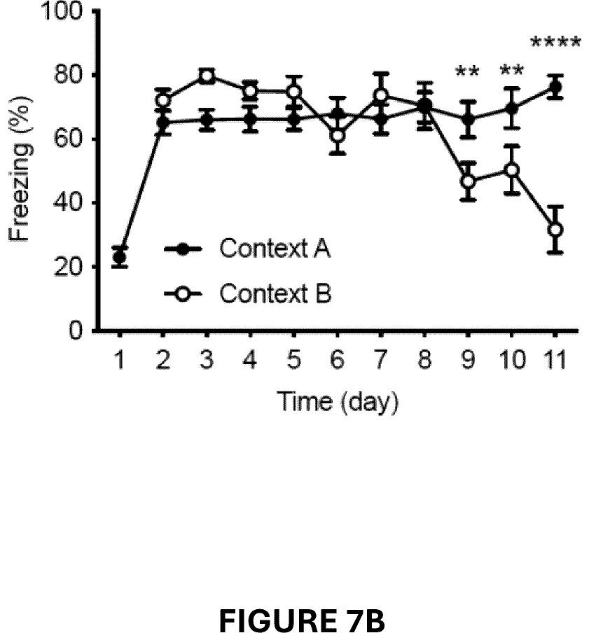

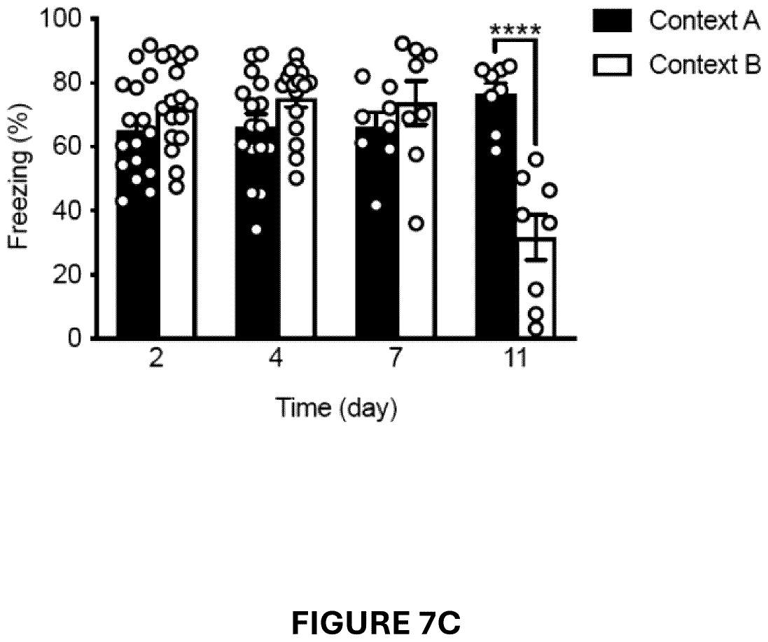

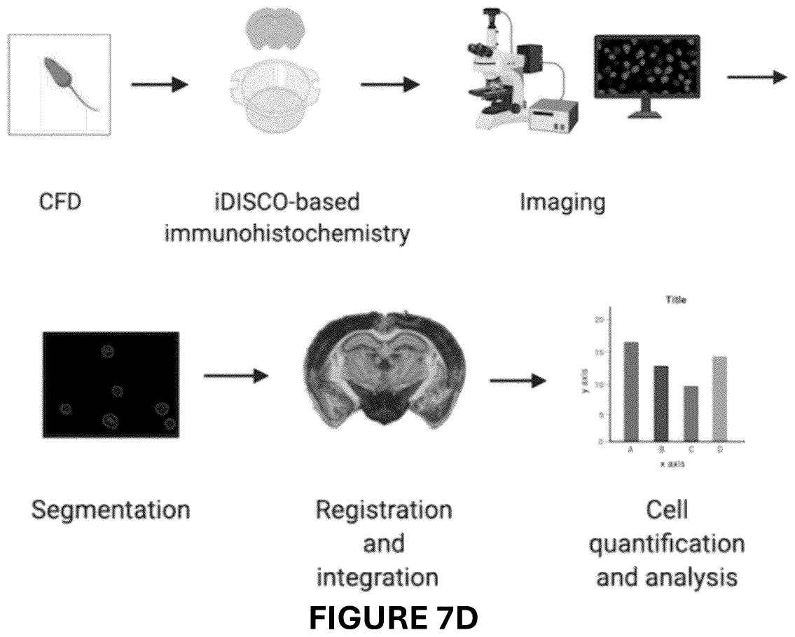

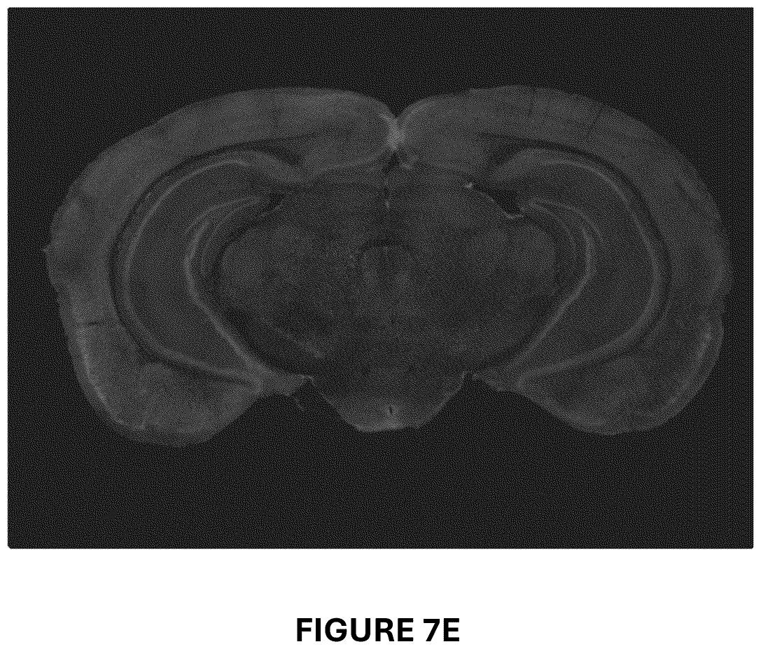

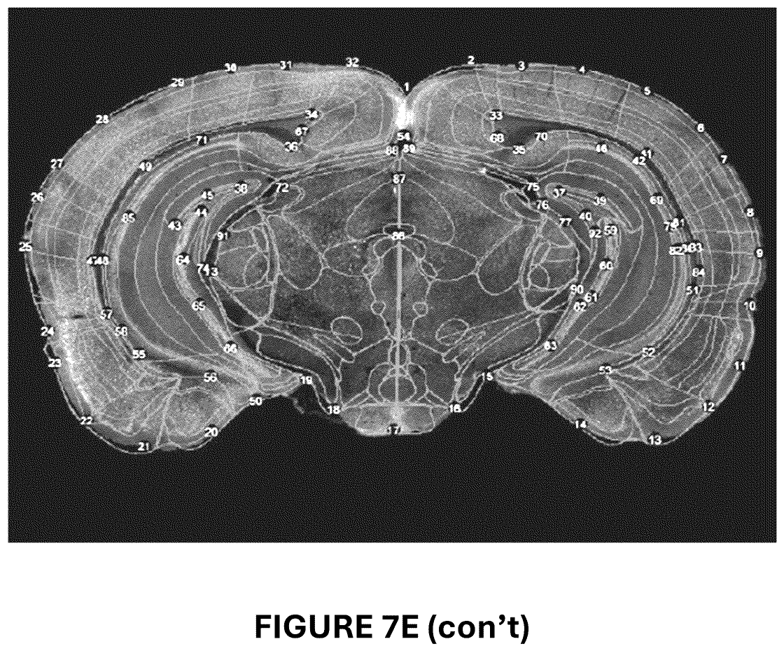

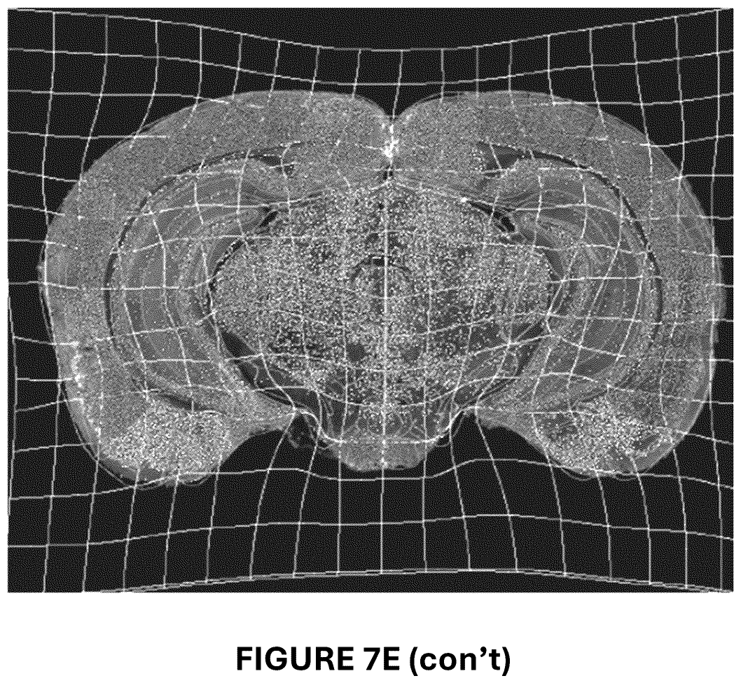

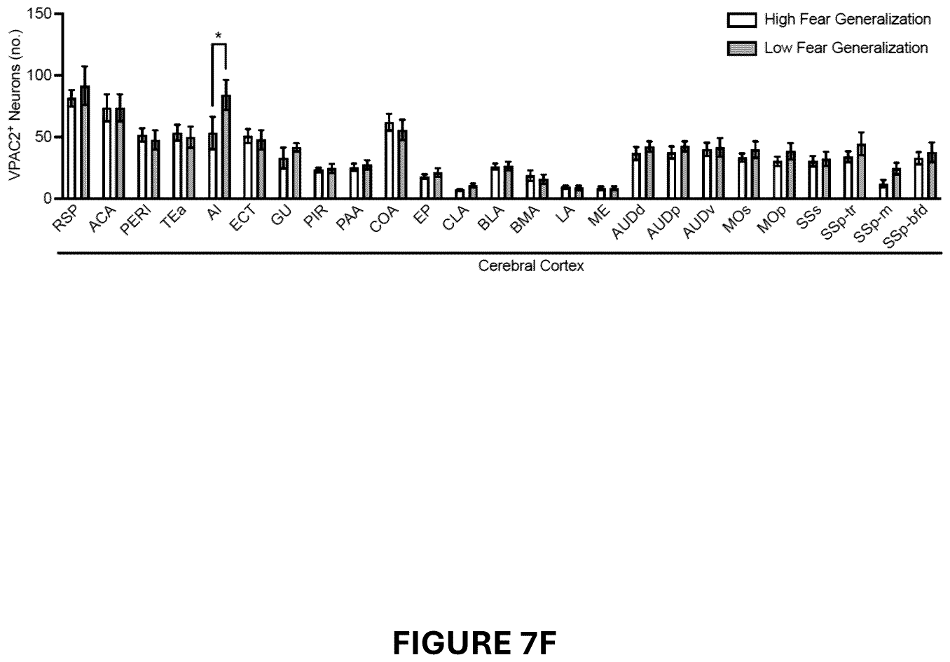

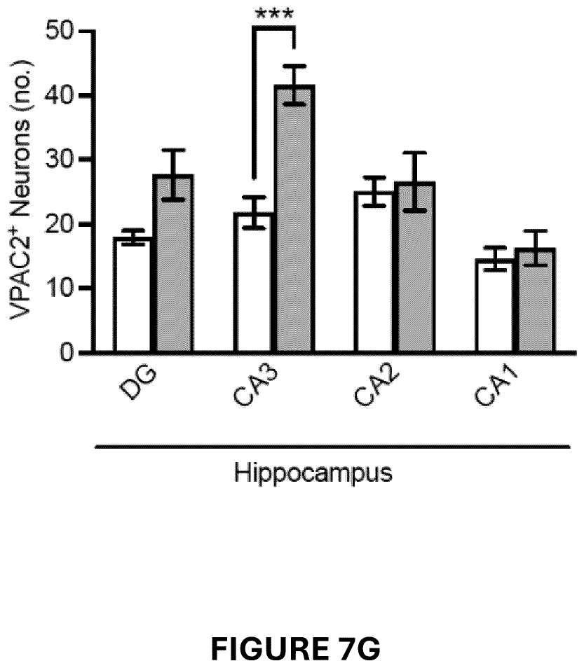

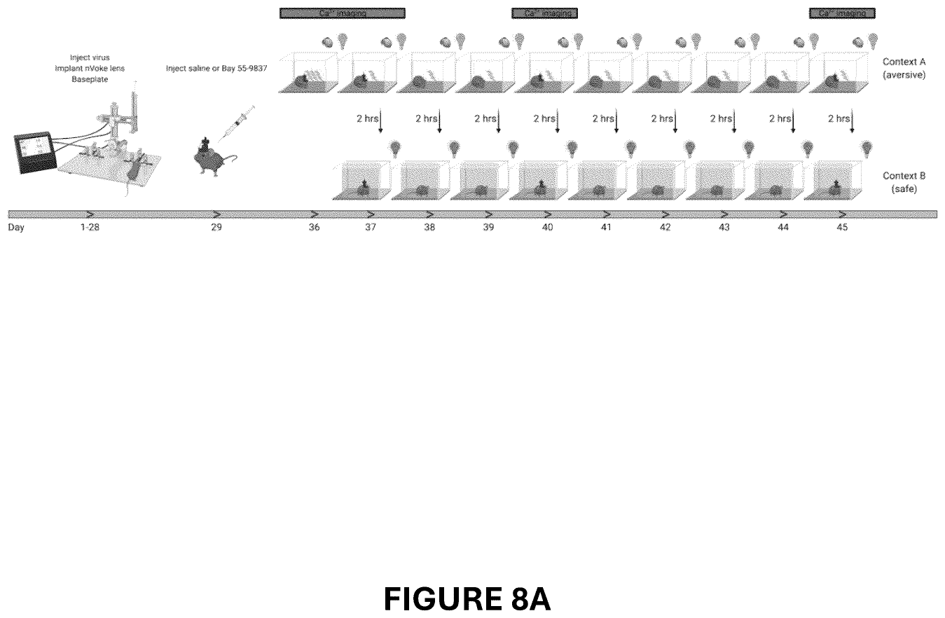

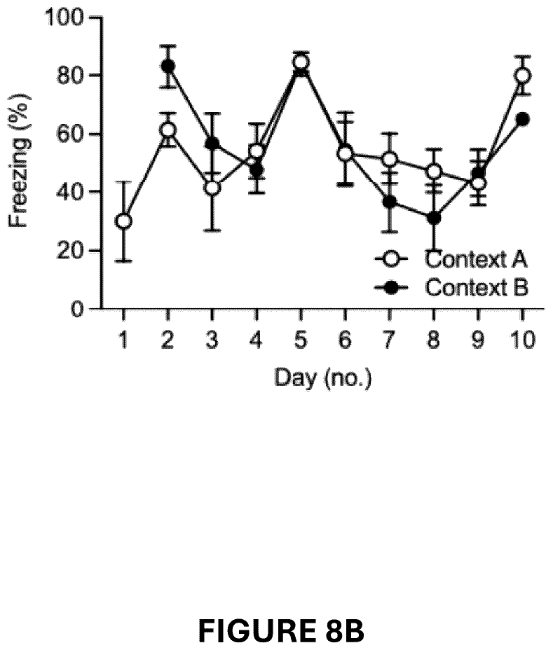

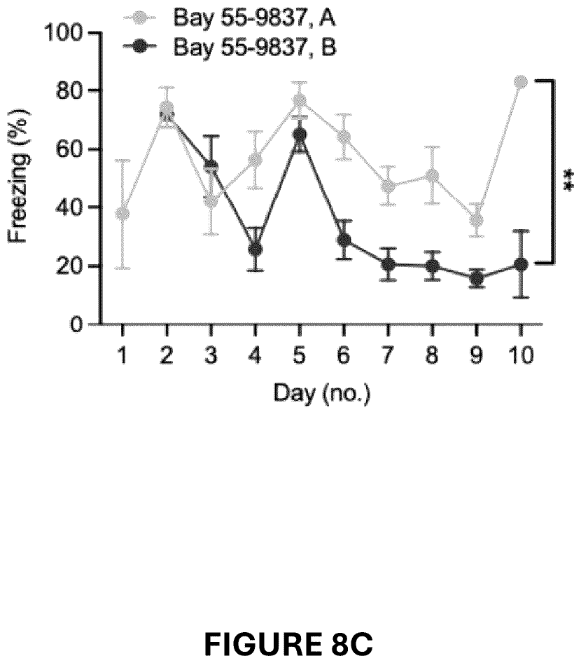

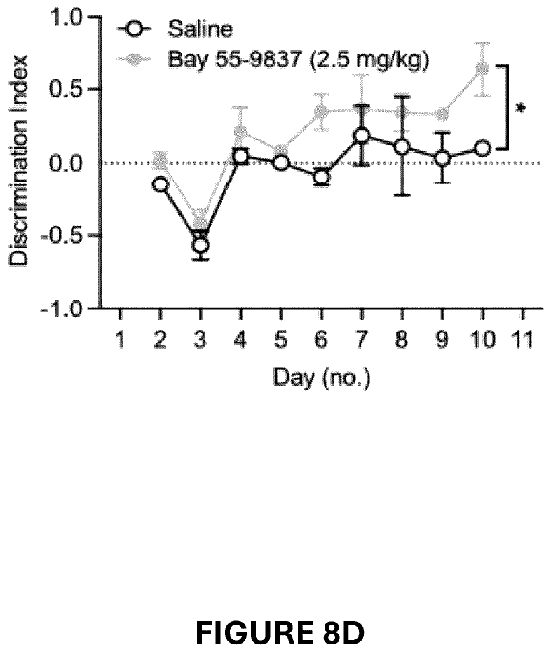

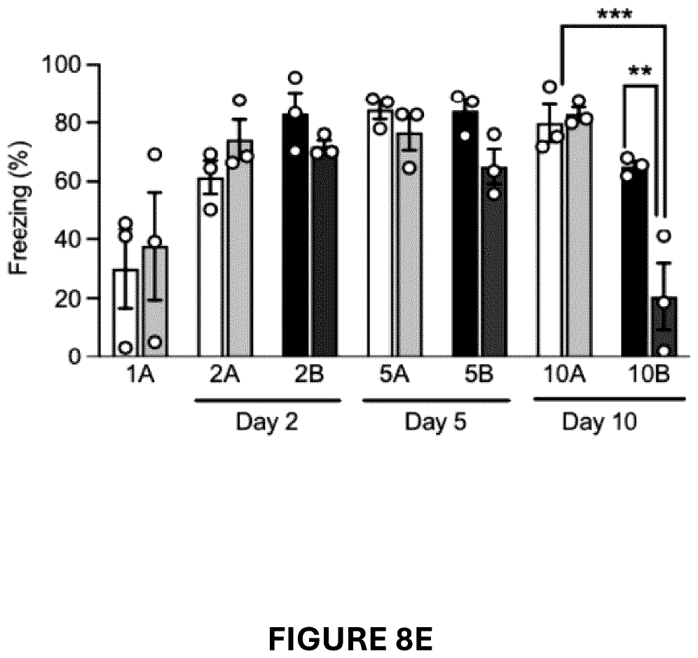

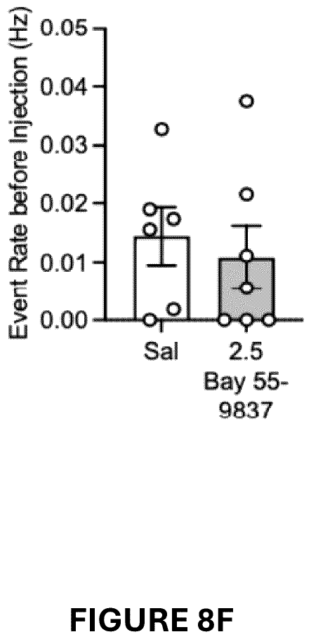

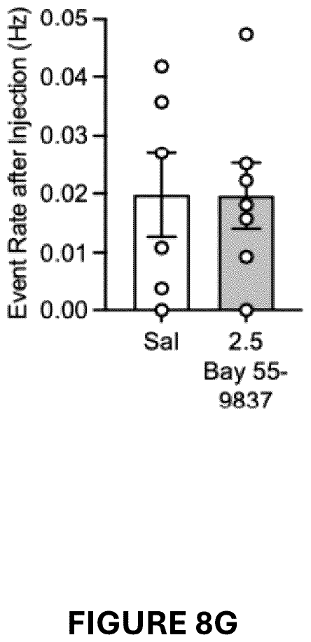

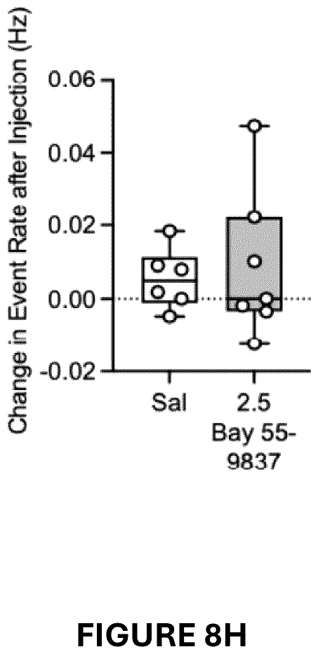

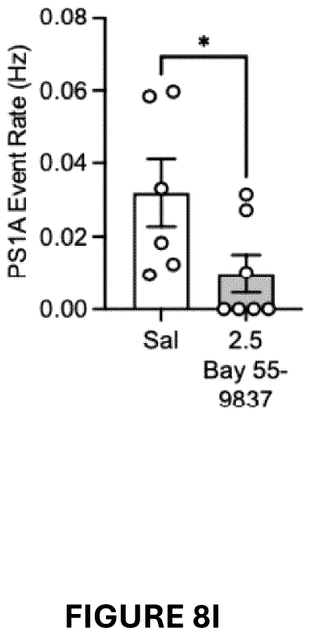

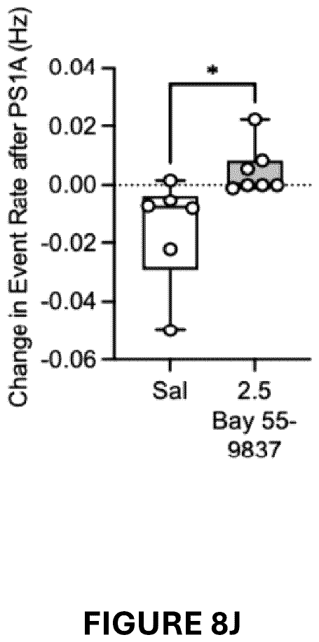

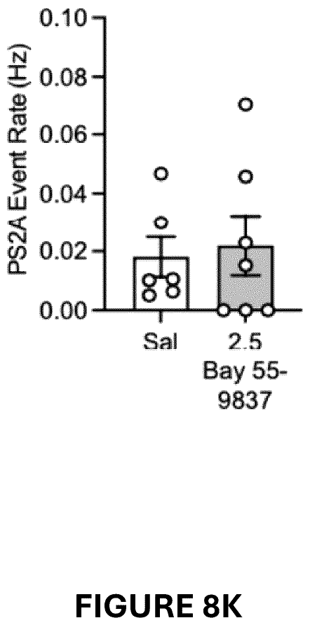

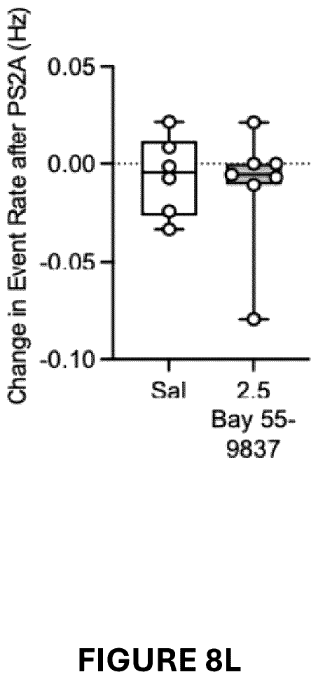

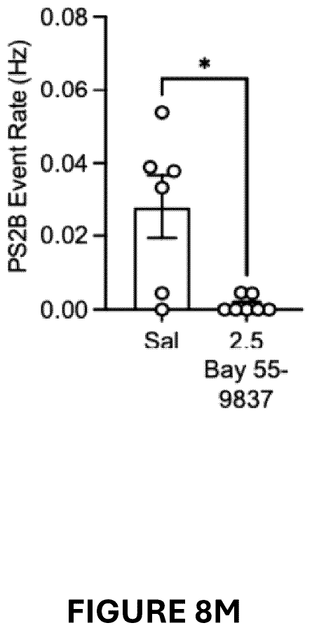

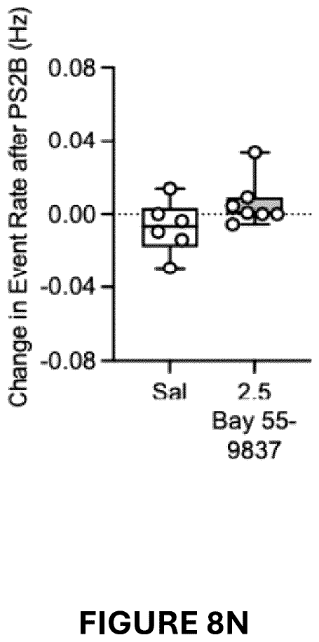

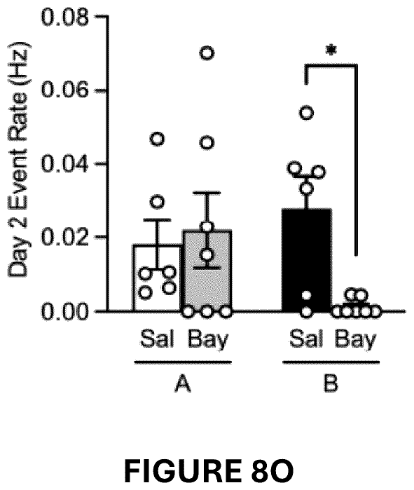

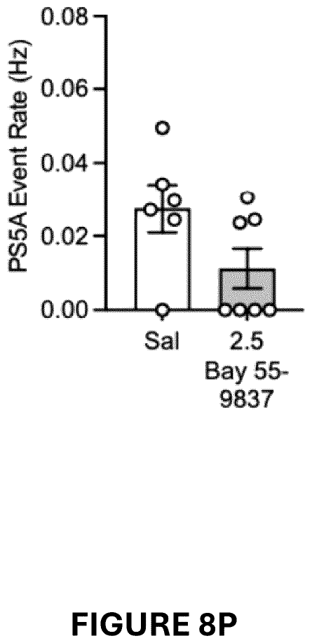

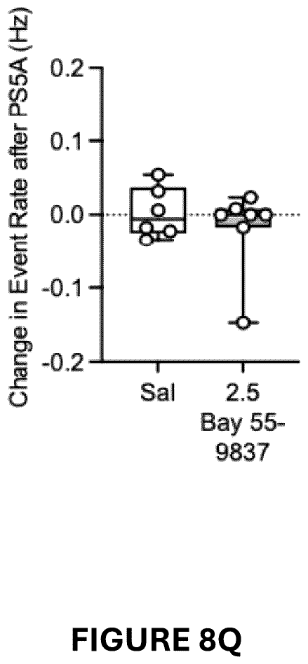

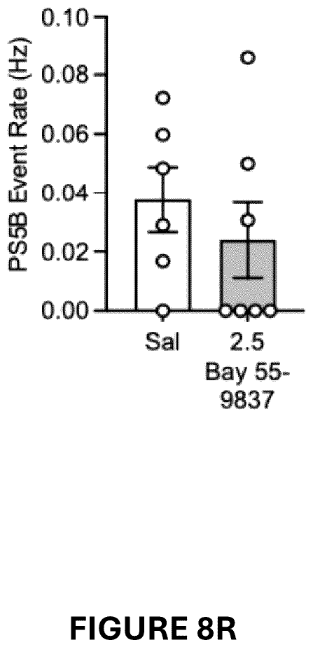

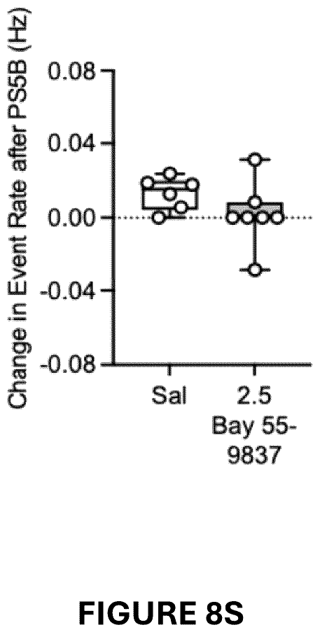

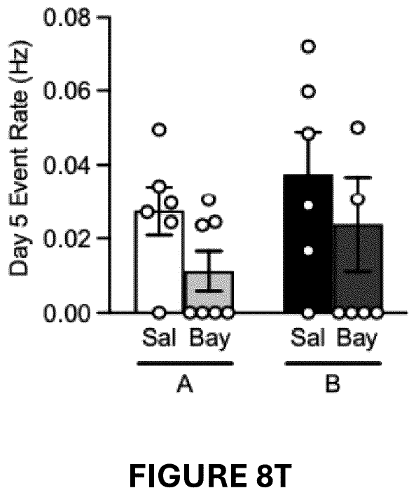

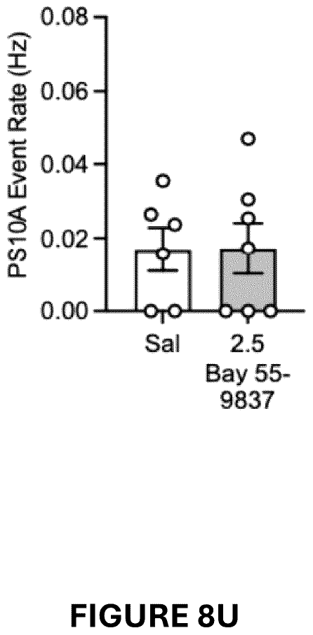

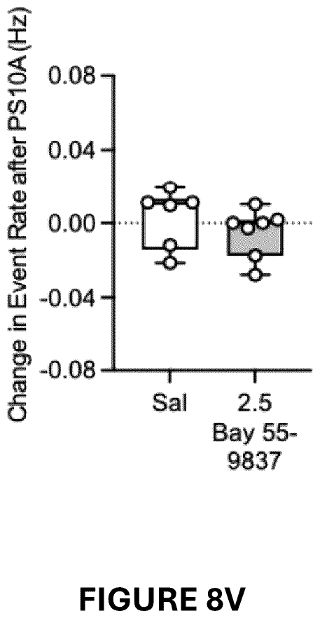

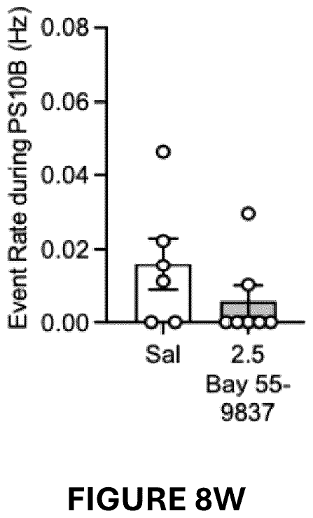

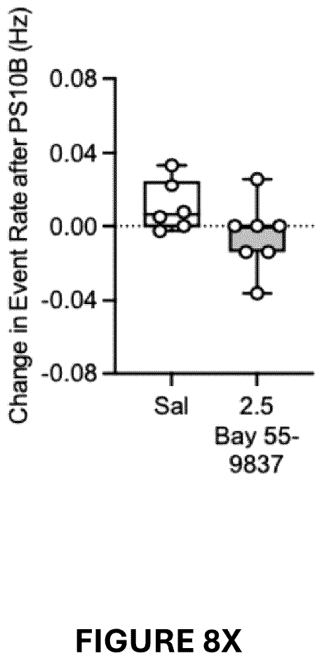

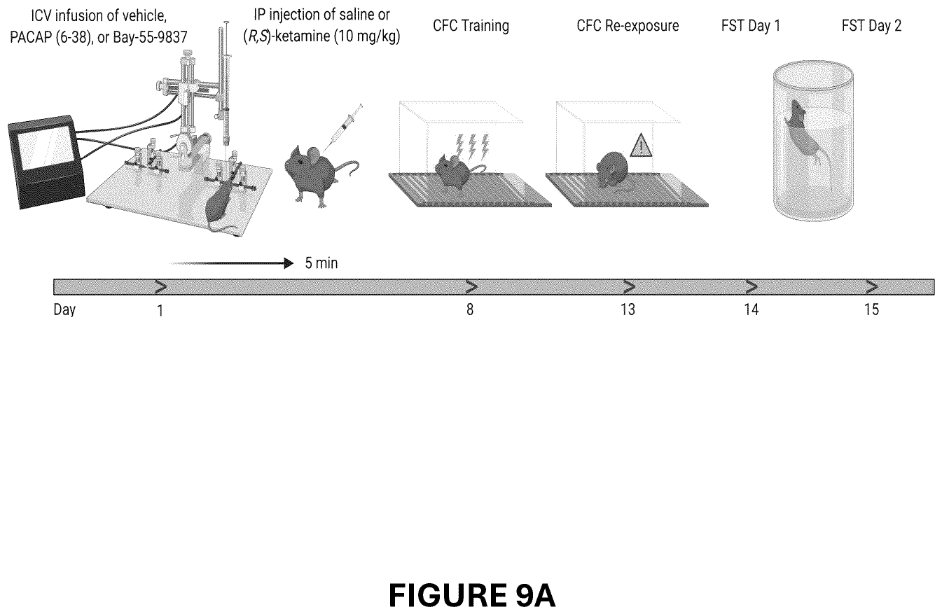

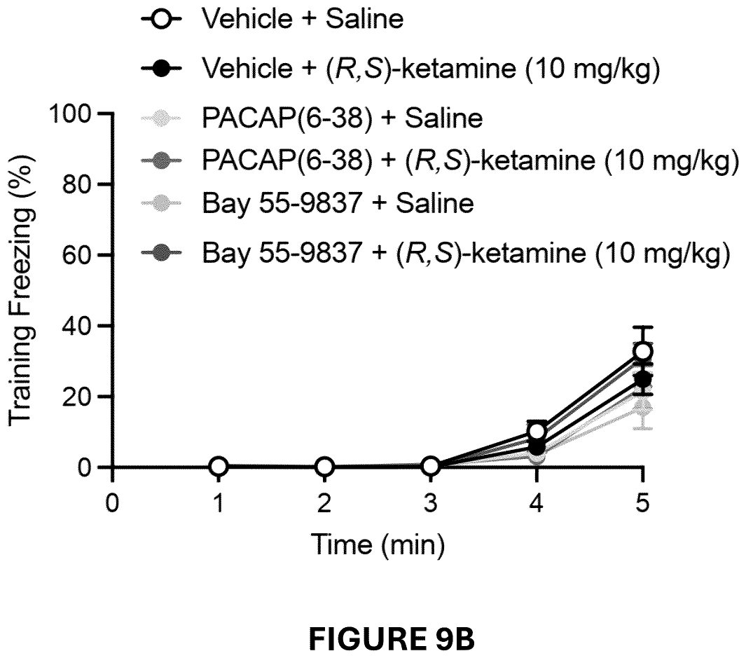

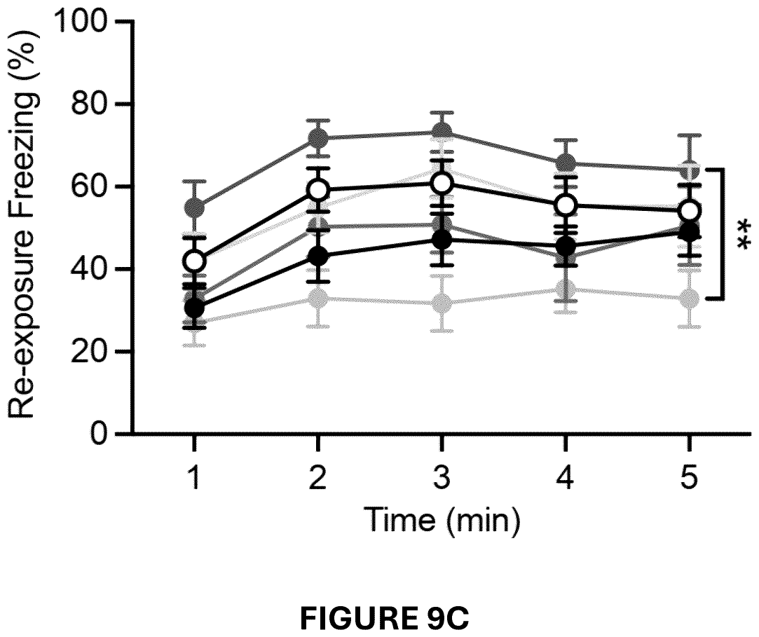

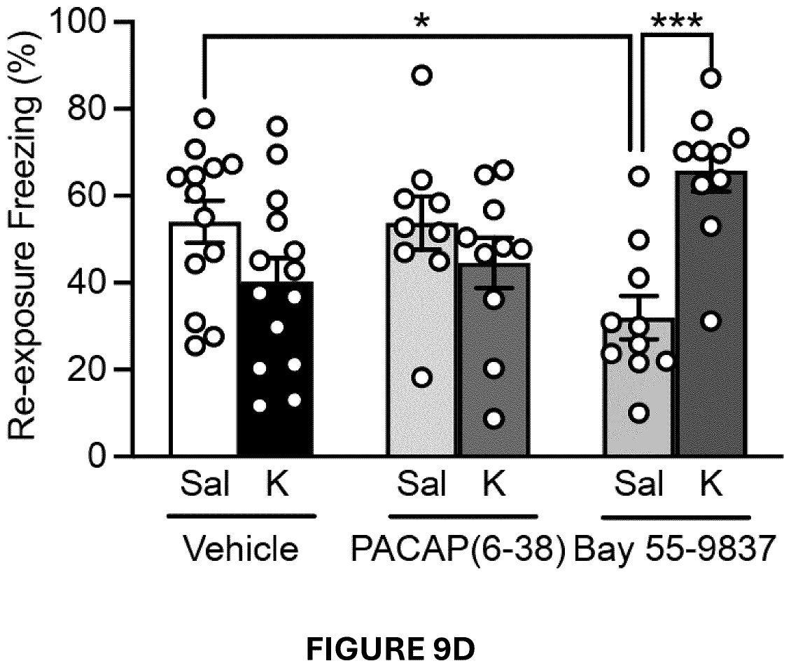

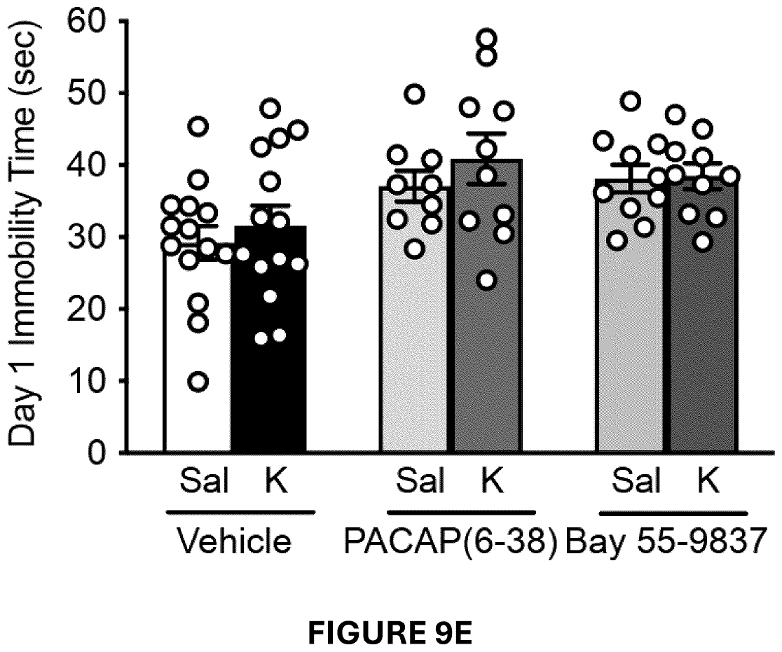

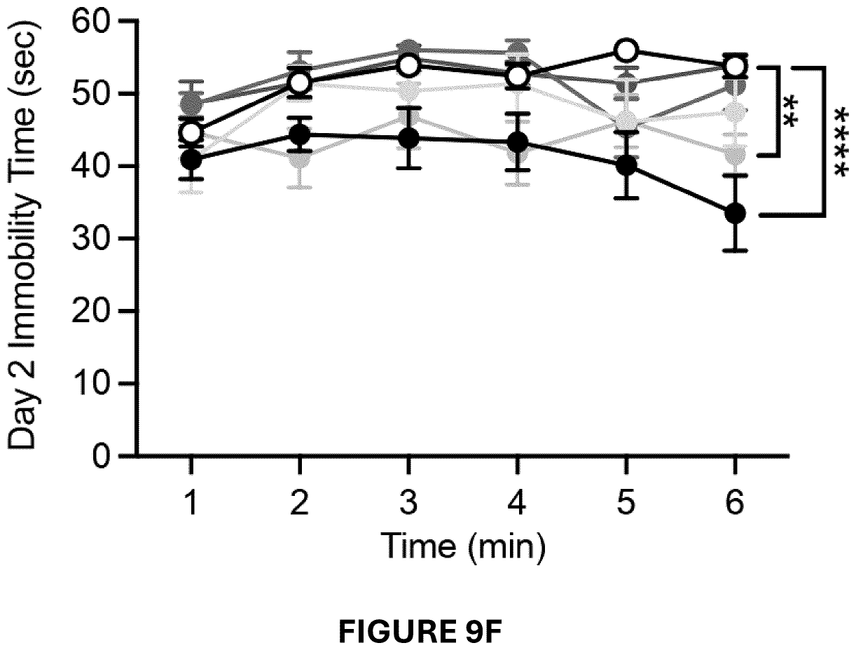

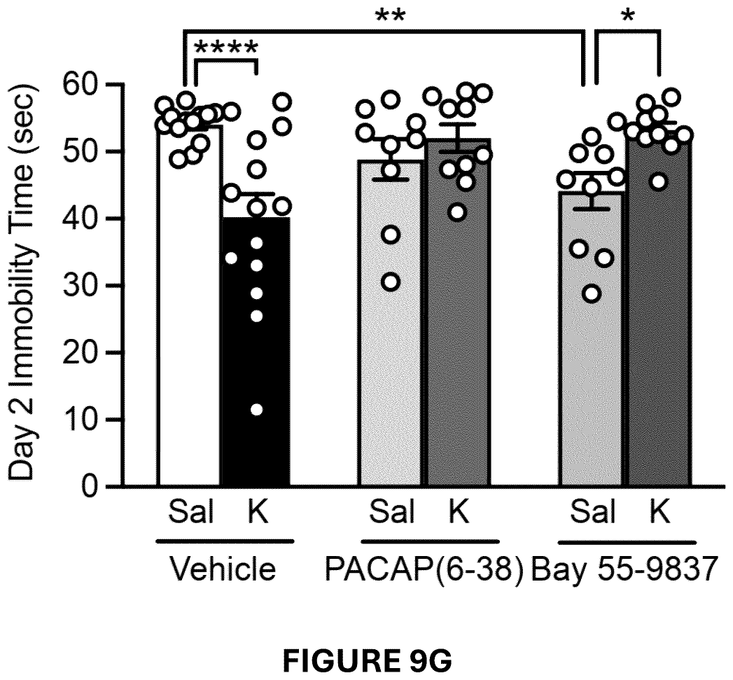

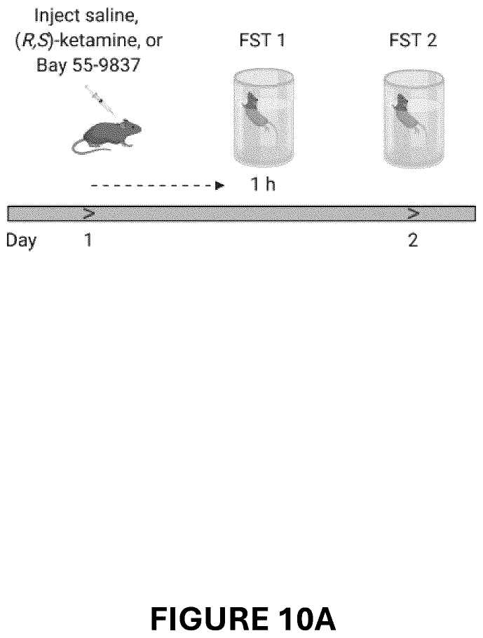

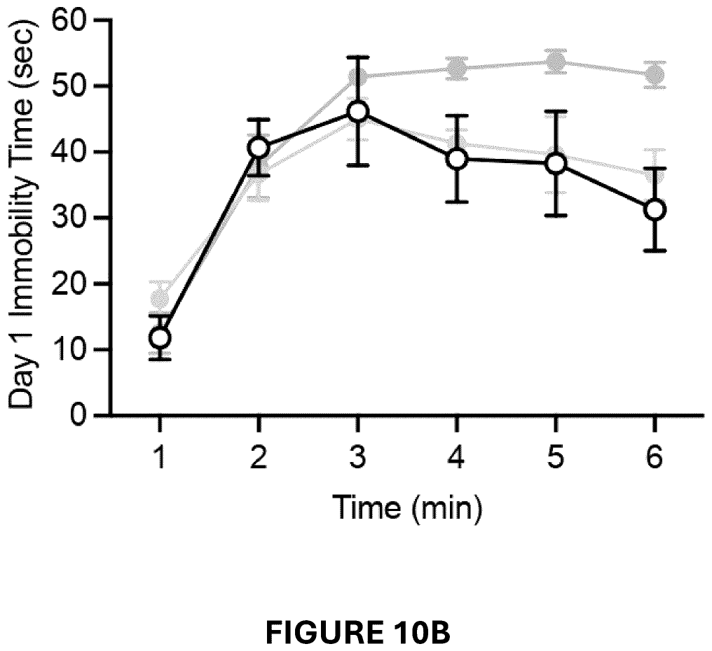

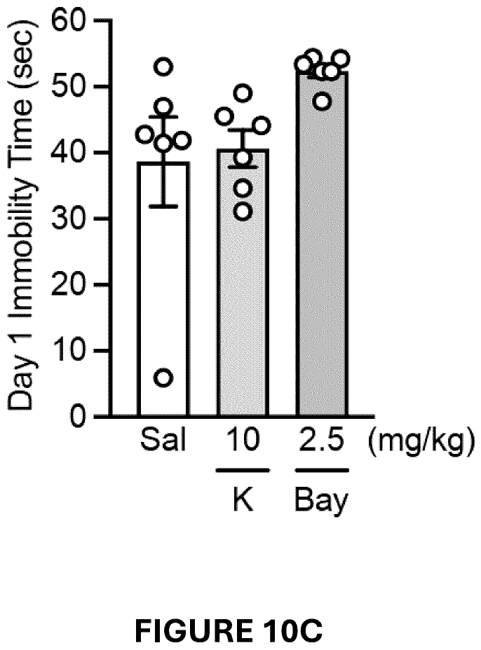

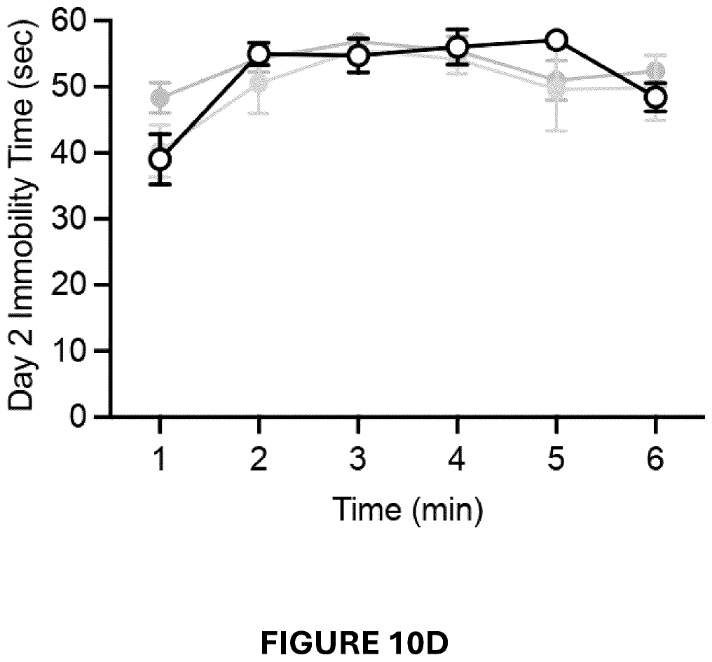

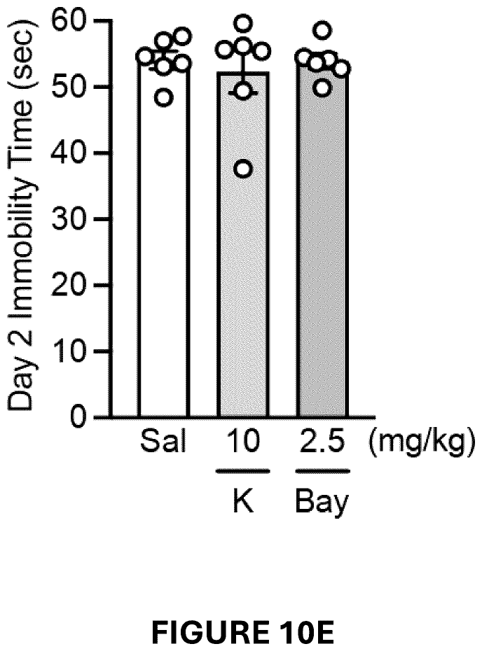

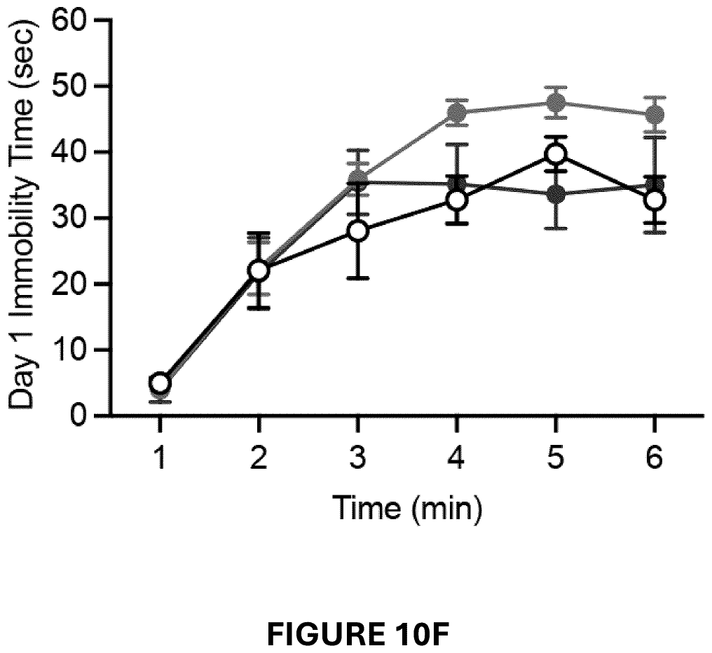

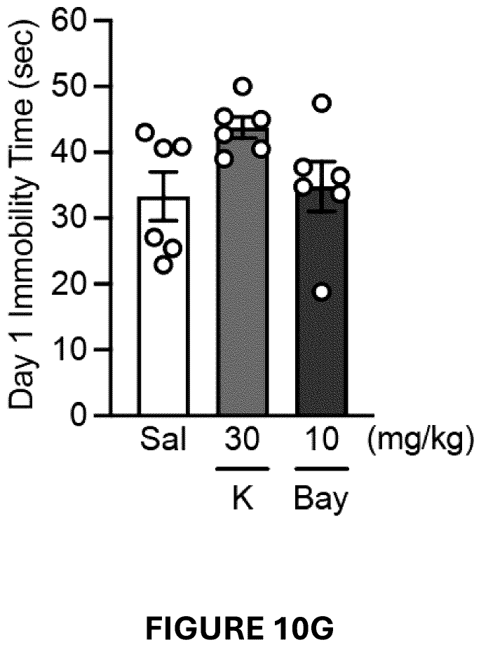

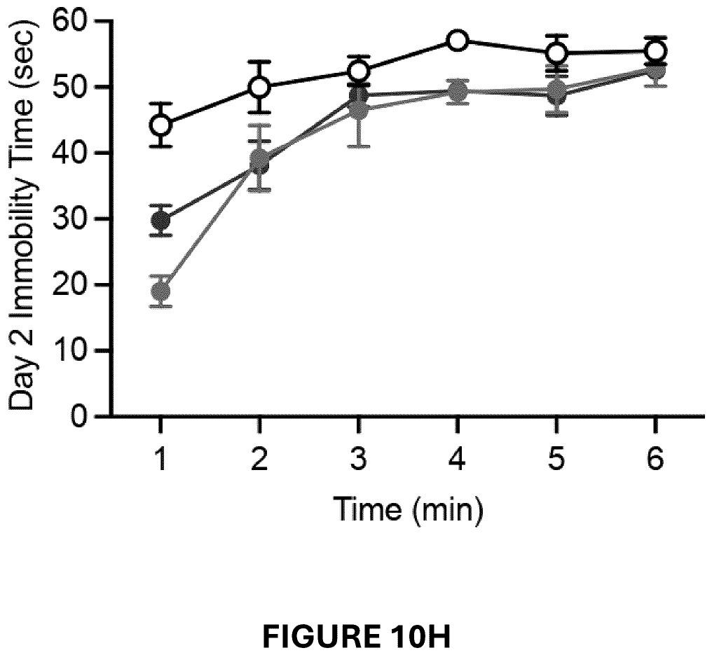

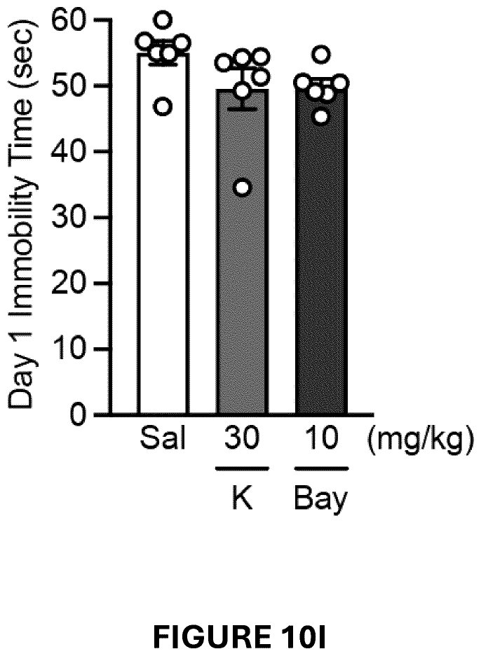

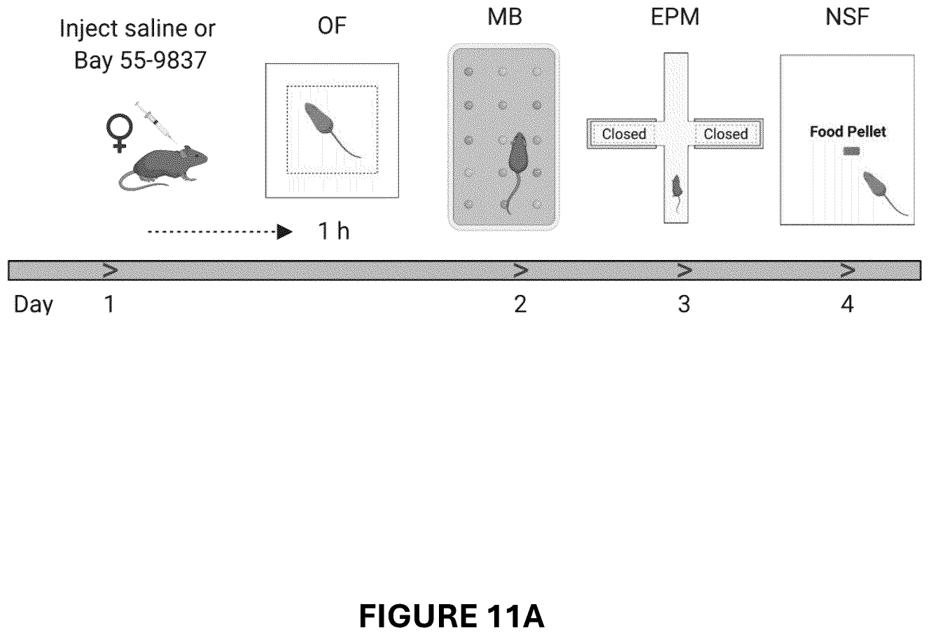

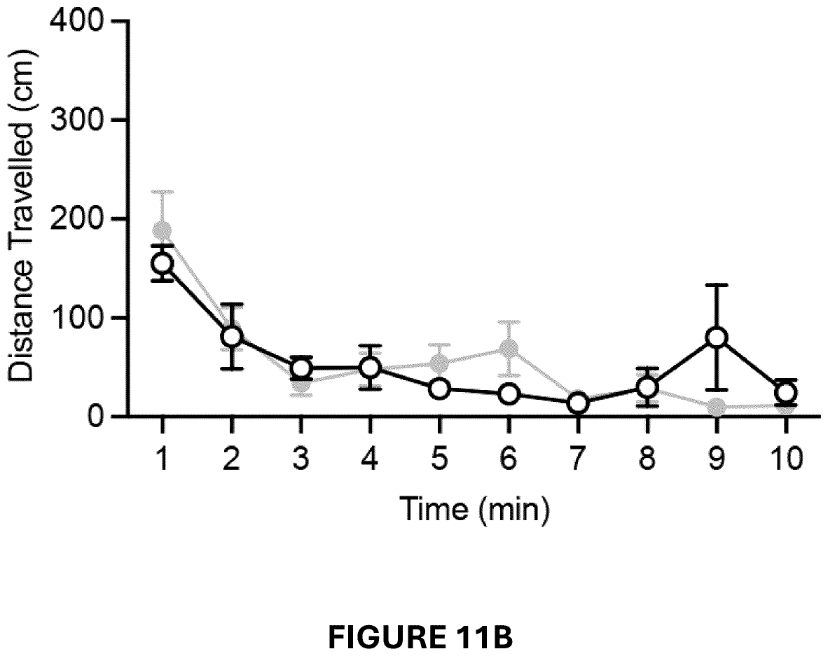

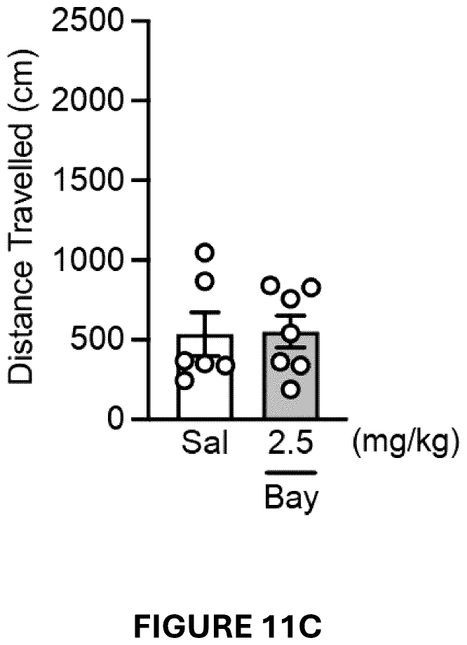

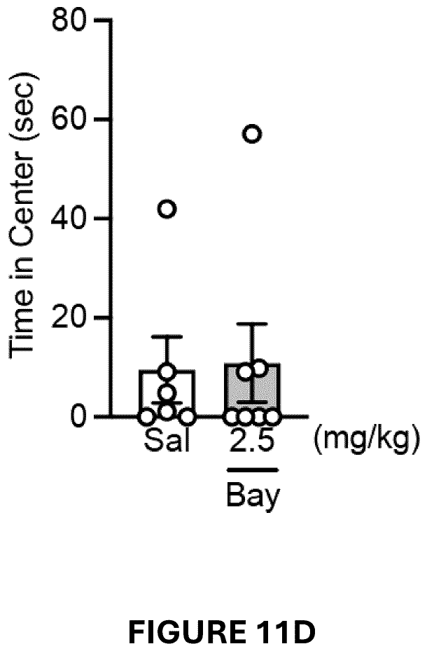

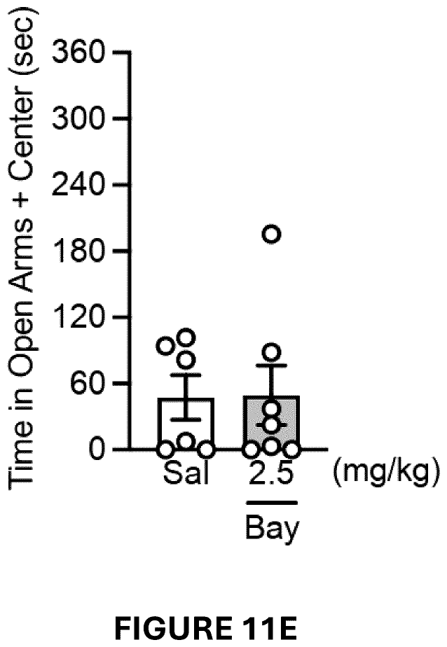

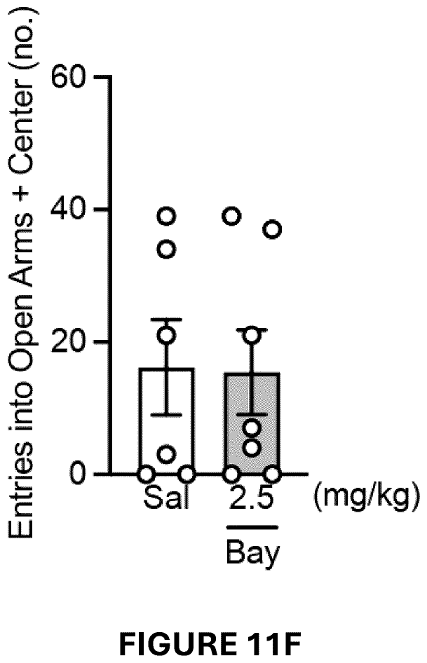

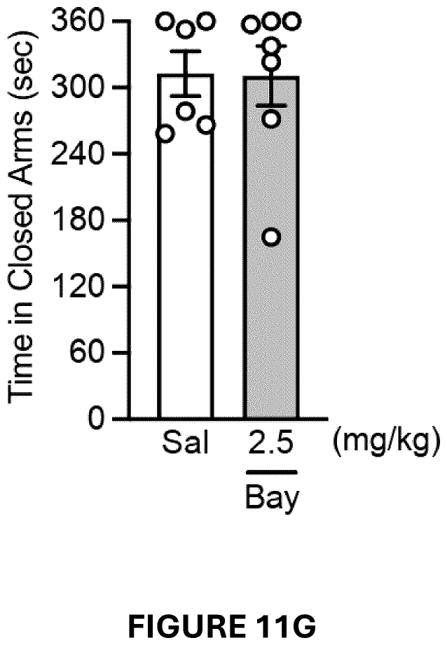

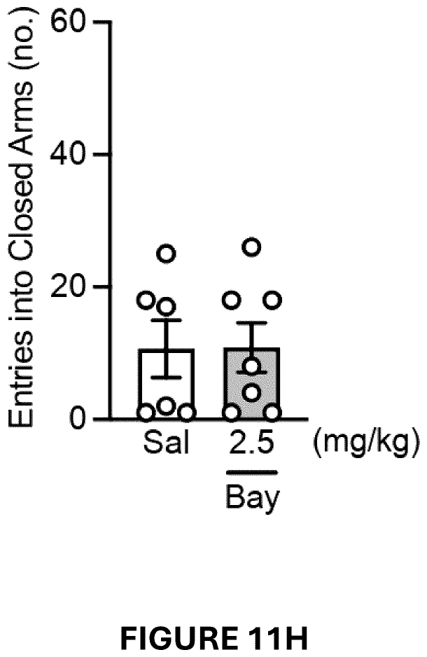

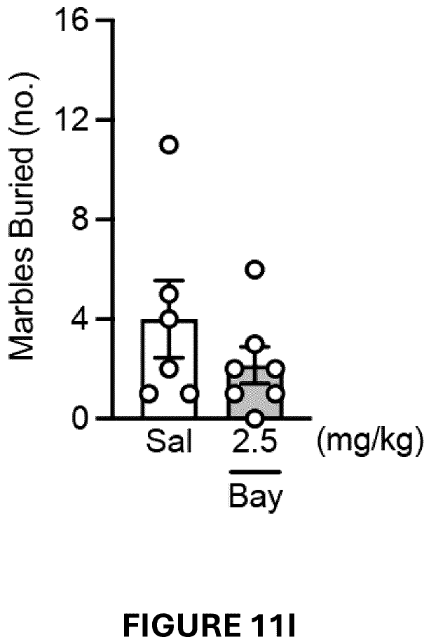

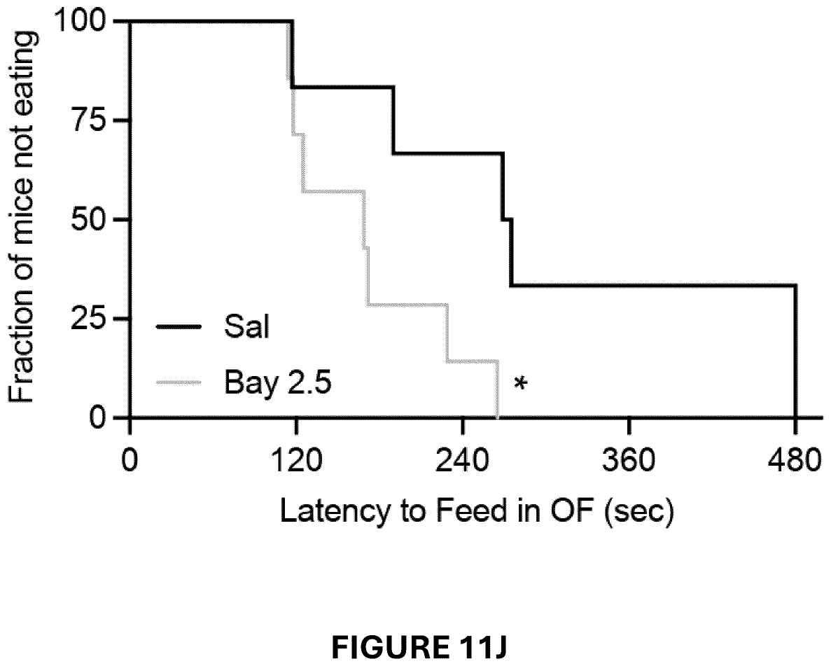

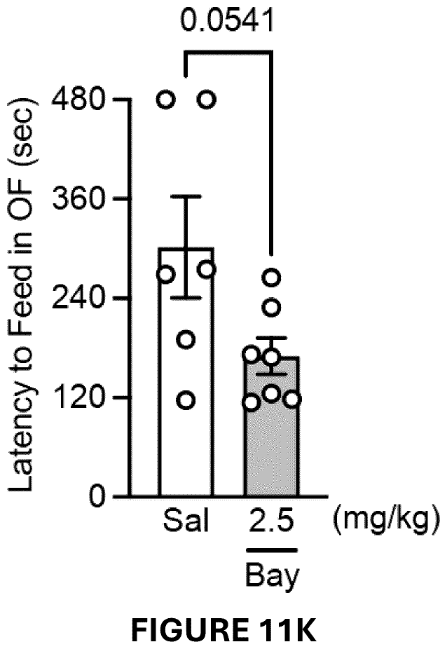

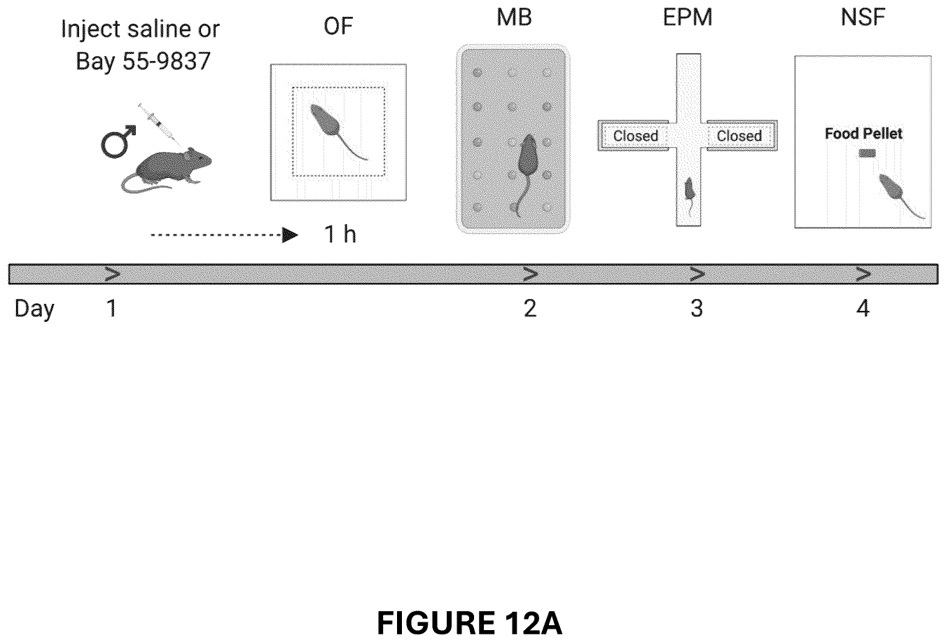

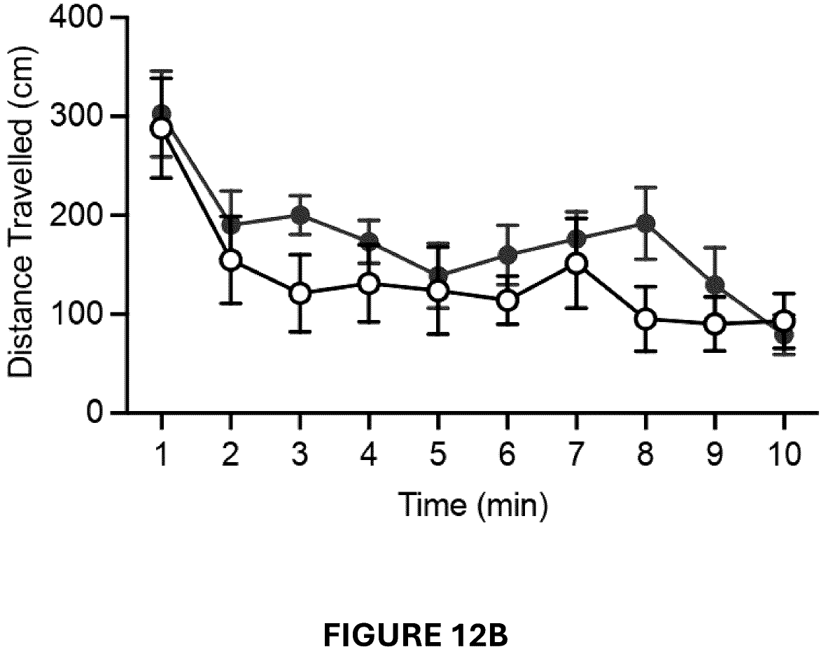

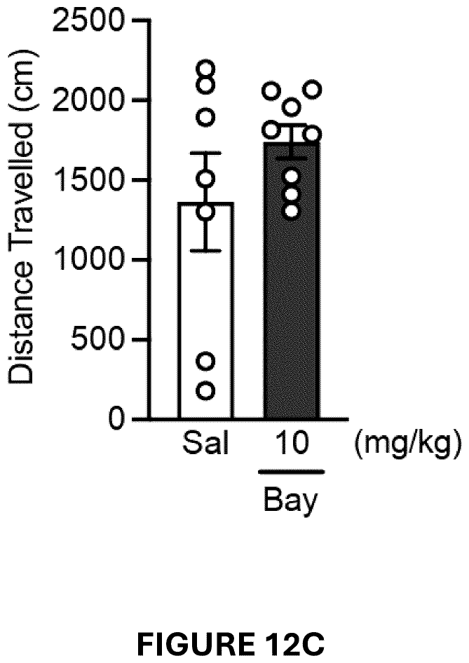

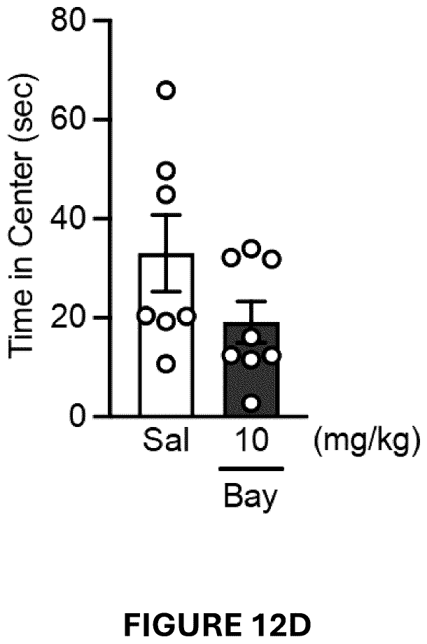

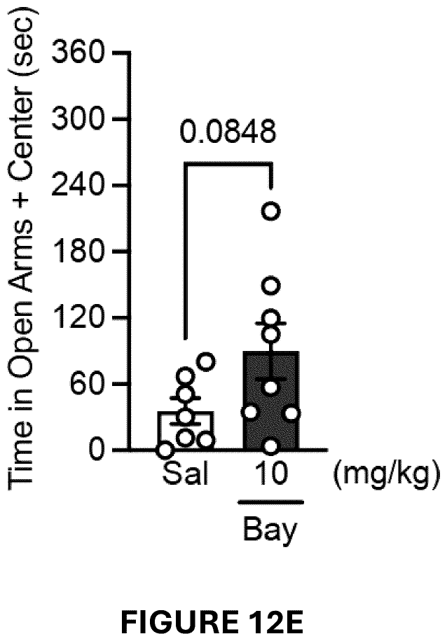

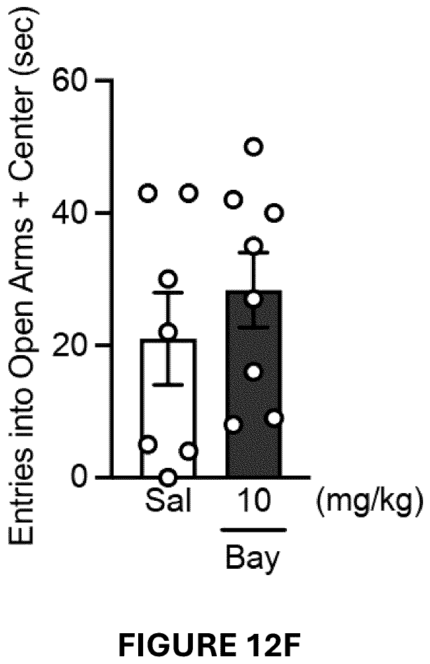

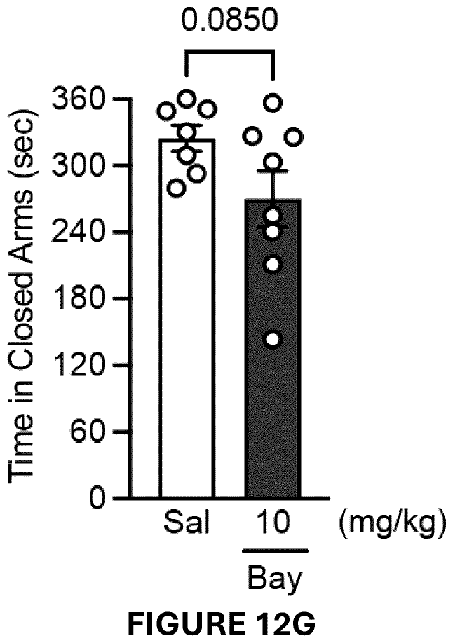

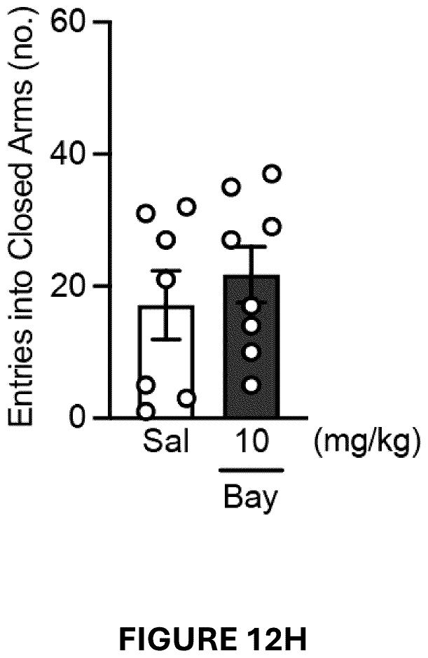

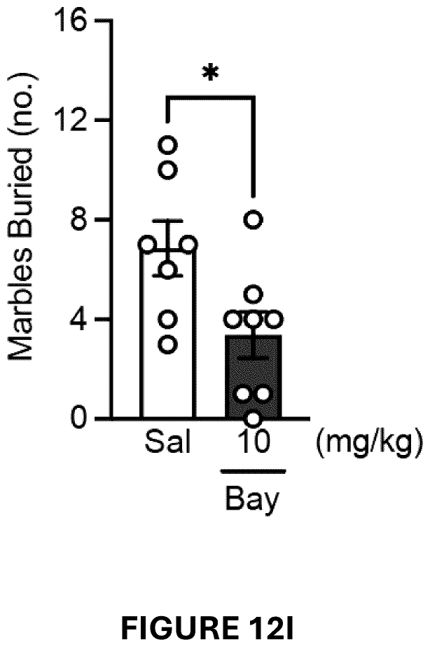

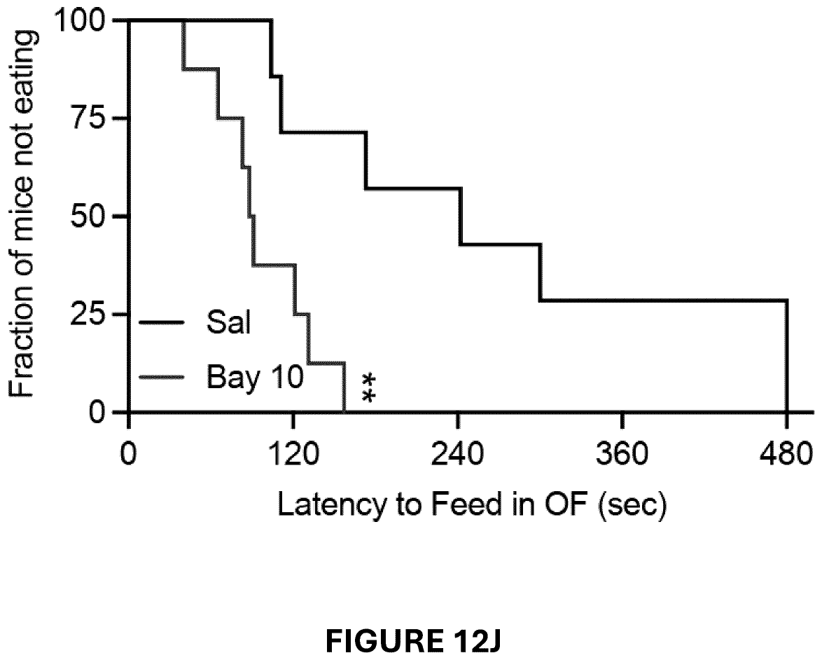

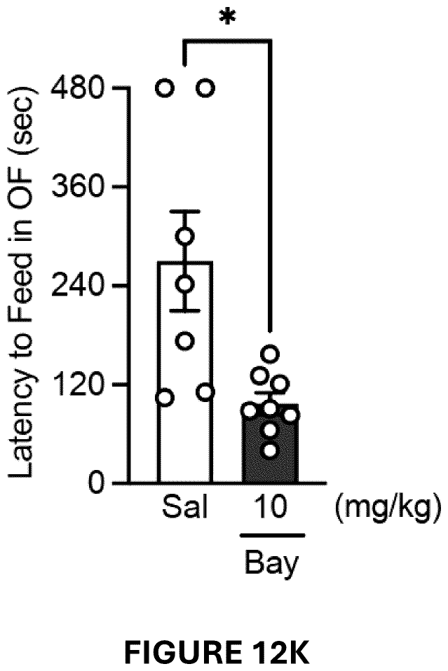

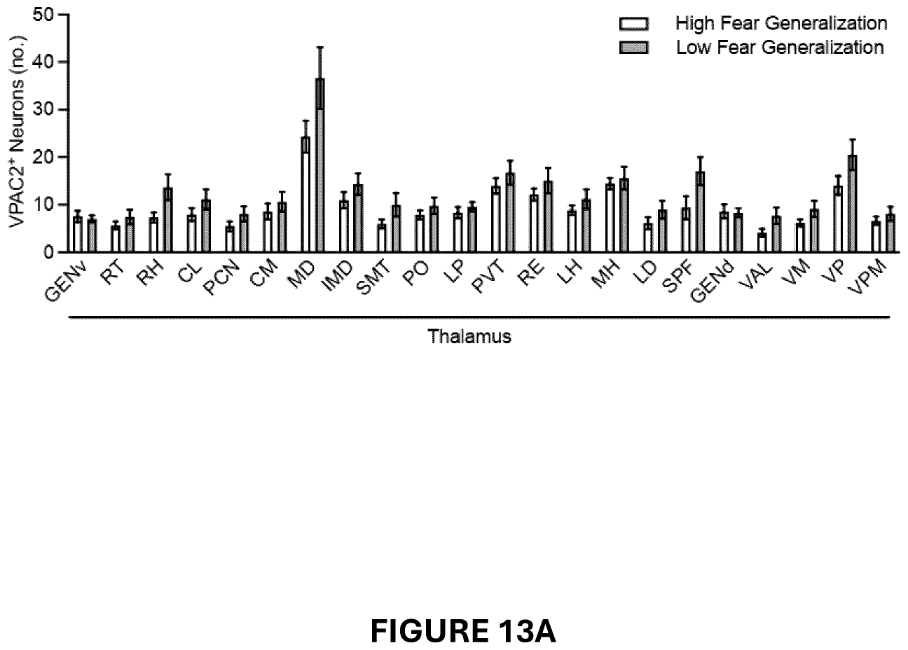

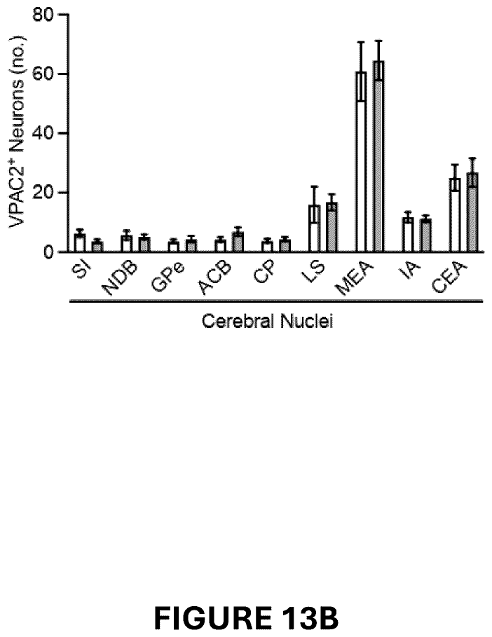

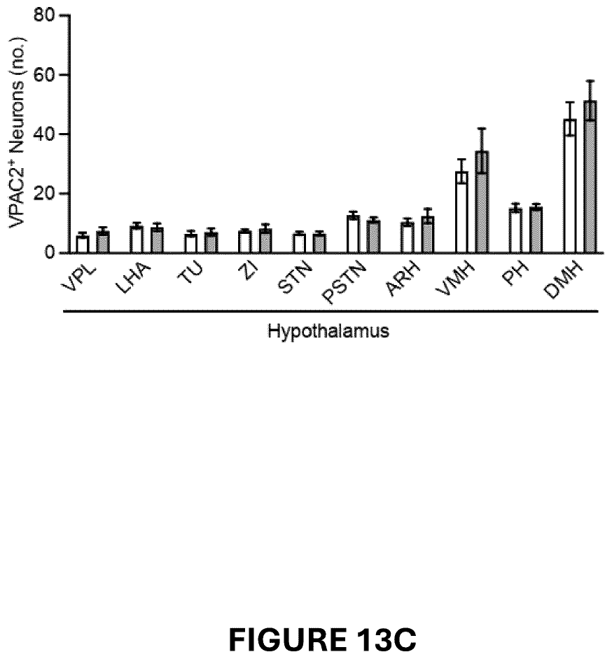

### DETAILED DESCRIPTION OF THE INVENTION

While the present invention may be embodied in many different forms, disclosed herein are specific illustrative embodiments thereof that exemplify the principles of the invention. It should be emphasized that the present invention is not limited to the specific embodiments illustrated. Moreover, any section headings used herein are for organizational purposes only and are not to be construed as limiting the subject matter described.

Unless otherwise defined herein, scientific, and technical terms used in connection with the present invention shall have the meanings that are commonly understood by those of ordinary skill in the art. Further, unless otherwise required by context, singular terms shall include pluralities and plural terms shall include the singular. More specifically, as used in this specification and the appended claims, the singular forms “a,” “an” and “the” include plural referents unless the context clearly dictates otherwise. Thus, for example, reference to “a protein” includes a plurality of proteins; reference to “a cell” includes mixtures of cells, and the like.

In addition, ranges provided in the specification and appended claims include both end points and all points between the end points. Therefore, a range of 1.0 to 2.0 includes 1.0, 2.0, and all points between 1.0 and 2.0.

The term “about” as used herein when referring to a measurable value such as an amount, a temporal duration, and the like, is meant to encompass variations of .+−.20%, .+−.10%, .+−.5%, .+−.1%, or .+−.0.1% from the specified value, as such variations are appropriate to perform the disclosed methods.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of “consisting essentially of,” when used in the claims, shall have its ordinary meaning as used in the field of patent law.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of and “consisting essentially of shall be closed or semi-closed transitional phrases, respectively.

Generally, nomenclature used in connection with, and techniques of, cell and tissue culture, molecular biology, immunology, microbiology, genetics and protein and nucleic acid chemistry and hybridization described herein are those well-known and commonly used in the art. The methods and techniques of the present invention are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well-known and commonly used in the art.

The inventions described herein relate to compositions and methods which prevent and protect against all three types of stress-induced maladaptive behaviors: fear, depressive-like, and anxiety-like behavior, including but not limited to major depressive disorder (MDD) and posttraumatic stress disorder (PTSD) as described hereinbelow.

### Stressors

A stressor is a stimulus that causes stress. It can be an event or other factor that disrupts the body's homeostasis of temperature, blood pressure, and/or other functions. In certain embodiments, a stressor is a traumatic or stressful event. Because humans have sophisticated brains and thought processes, anticipating a disruption can also be a stressor. In certain embodiments, a stressor is injury, trauma, combat, warfare, surgery, an accident, a criminal assault, child abuse, natural or human-caused disasters, a crash, grief, hunger, heat, cold, chemical exposure, autoimmune disease, infectious disease, viral infection, cancer, exhaustion, physical distress, neuropathy, hyperalgesia, allodynia, emotional distress, or depression. A traumatic event may be an event or something that threatens the person's life or the life of a close one or it could be something witnessed. A stressor may be acute, or may be chronic.

There are numerous physiological processes that are altered in response to stress. Among these are altered cortisol, corticotropin, catecholamine and serotonin levels. These levels return to baseline after an acute stressor is removed. These biochemical markers of stress in turn lead to ill health and psychosocial disorders. Consequently, stress plays a major role in physical and mental health. Stress can affect the onset of, or susceptibility to disease. It can also affect the progression or course of disease even when there is another underlying pathophysiology of the disease. Recovery from an existing disease can also be delayed due to stress. For example, stress is a contributing factor to high blood pressure, heart disease, headaches, colitis, irritable bowel syndrome, temporo-mandibular joint disorder, cancer, peptic ulcers, insomnia, skin disorders and asthma. Stress can also aggravate other conditions such as multiple sclerosis, diabetes, herpes, mental illness, substance abuse and psychiatric disorders characterized by the presence of violent or aggressive tendencies. Particularly, stress contributes to functional somatic disorders, affective disorders, and major depressive disorder (MDD). These include disorders such as chronic fatigue syndrome (CFS), fibromyalgia (FMS), Gulf War Syndrome, anxiety, and post-traumatic stress disorder (PTSD).

In certain embodiments, a subject may be administered a VPAC2 agonist prior to a situation in which the subject is likely to be exposed to traumatic stress, immediately after exposure to traumatic stress, and/or when the subject feels that his or her PTSD symptoms are likely to appear. Exemplary embodiments of such use include administration prior to military deployment to protect service members (active combat soldiers, battlefield surgeons, etc.). Potential non-military use cases include, but are not limited to police, firefighters, first responders, emergency medical technicians (EMTs), emergency room (ER) doctors, prison guards (and prisoners), humanitarian aid workers, and refugees.

Resilience to stress refers to the capacity of a subject to adapt or change successfully, and/or to maintain physiological, neurological, or psychological homeostasis, in the face of a stressor (e.g., adversity). As used herein, the term “enhancing resilience” refers to increasing the ability of a subject to experience a stressor (e.g., a traumatic event) without suffering a stress-induced affective disorder, and/or with less post-event symptomatology or disruption of homeostasis and/or normal activities of daily living. In certain embodiments, improving resilience can prevent a stress-induced affective disorder. In certain embodiments, improving resilience can reduce at least one of the signs, symptoms, or symptom clusters of a stress-induced affective disorder. In certain embodiments, administration of a VPAC2 agonist enhances a subject's resilience to stress, helps protect against developing stressor-related psychopathology, decrease the functional consequences of stressor-induced disorders (e.g., PTSD, etc.), and reduce medical morbidity and mortality.

The Connor-Davidson Resilience Scale (CD-RISC) is a 25-item self-report scale, each rated on a 5-point scale (0-4), with higher scores reflecting greater resilience. Resilience, psychological growth, and life satisfaction may be measured with the CD-RISC, the Purpose in Life Scale, the abbreviated MOS Social Support Survey, the PTGI, and the Q-LES-Q.

### Stress-Induced Affective Disorders

There are numerous disorders that are either caused by or exacerbated by stress. The compositions and methods disclosed herein can prevent or delay a stress-induced affective disorder or stress-induced psychopathology. Stress-induced affective disorders or stress-induced psychopathologies which may be prevented or treated by the present compositions and methods include, but are not limited to addictive disorders such as substance abuse, anorexia, bulimia, obesity, smoking addiction, and weight addiction; anxiety disorders such as agoraphobia, anxiety disorder, obsessive compulsive disorder, panic attacks, performance anxiety, phobias, and post-traumatic stress disorder (PTSD); psychiatric disorders such as stress-induced psychiatric disorders; autoimmune diseases such as allergies, arthritis, fibromyalgia, fibromytosis, lupus, multiple sclerosis, rheumatoid arthritis, Sjogren's syndrome, and vitiligo; cancer such as bone cancer, brain cancer, breast cancer, cervical cancer, colon cancer, Hodgkin's disease, leukemia, liver cancer, lung cancer, lymphoma, multiple myeloma, ovarian cancer, pancreatic cancer, and prostate cancer; cardiovascular disorders such as arrhythmia, arteriosclerosis, Burger's disease, essential hypertension, fibrillation, mitral valve prolapse, palpitations, peripheral vascular disease, Raynaud's disease, stroke, tachycardia, and Wolff-Parkinson-White Syndrome; and developmental disorders such as attention deficit disorder, concentration problems, conduct disorder, dyslexia, hyperkinesis, language and speech disorders, and learning disabilities.

### Anxiety Disorders

The compositions and methods disclosed herein can prevent or delay an anxiety disorder. The five major types of anxiety disorders are: panic disorder; obsessive-compulsive disorder; post-traumatic stress disorder; generalized anxiety disorder; and phobias (including social phobia, also called social anxiety disorder). Each anxiety disorder has its own distinct features, but they are all bound together by the common theme of excessive, irrational fear and dread. It is common for an anxiety disorder to accompany depression, eating disorders, substance abuse, or another anxiety disorder.

Panic disorder is characterized by repeated episodes of intense fear that strike often and without warning. Physical symptoms include chest pain, heart palpitations, shortness of breath, dizziness, abdominal distress, feelings of unreality, and fear of dying. Obsessive-compulsive disorder is characterized by repeated, unwanted thoughts or compulsive behaviors that seem impossible to stop or control. Generalized Anxiety Disorder is characterized by exaggerated worrisome thoughts and tension about everyday routine life events and activities, lasting at least six months. Almost always anticipating the worst even though there is little reason to expect it; accompanied by physical symptoms, such as fatigue, trembling, muscle tension, headache, or nausea. Phobias are characterized into two major types of phobias, social phobia and specific phobia. People with social phobia have an overwhelming and disabling fear of scrutiny, embarrassment, or humiliation in social situations, which leads to avoidance of many potentially pleasurable and meaningful activities. People with specific phobia experience extreme, disabling, and irrational fear of something that poses little or no actual danger; the fear leads to avoidance of objects or situations and can cause people to limit their lives unnecessarily.

### Posttraumatic Stress Disorder (PTSD)

Typically, a subject suffering from PTSD was exposed to a traumatic event in which the person experienced, witnessed, or was confronted with an event or events that involved actual or threatened death or serious injury, or a threat to the physical integrity of self or others and the person's response involved intense fear, helplessness, or horror.

Having repeated intrusive memories of the trauma exposure is one of the core symptoms of PTSD. Patients with PTSD are known to display impairments in learning and memory during neuropsychological testing. Other core symptoms of PTSD include heightened stress sensitivity (startle), tension and anxiety, memory disturbances, and dissociation.

In certain embodiments, the compositions and methods disclosed herein prevent or inhibit the development of PTSD in a subject. In certain embodiments, the present compositions and methods prevent or inhibit the development of one or more PTSD-like symptoms. In certain embodiments, a subject may be administered a VPAC2 agonist prior to a situation in which the subject (such as an early responder or military personnel) is likely to be exposed to traumatic stress, immediately after exposure to traumatic stress, and/or when the subject feels that his or her PTSD symptoms are likely to appear.

Typically, the traumatic event is persistently re-experienced in one or more of the following ways: recurrent and intrusive distressing recollections of the event, including images, thoughts, or perceptions; recurrent distressing dreams of the event; acting or feeling as if the traumatic event were recurring (includes a sense of reliving the experience, illusions, hallucinations, and dissociative flashback episodes, including those that occur on awakening or when intoxicated); intense psychological distress at exposure to internal or external cues that symbolize or resemble an aspect of the traumatic event; and physiological reactivity on exposure to internal or external cues that symbolize or resemble an aspect of the traumatic event.

An individual suffering from PTSD also has persistent avoidance of stimuli associated with the trauma and numbing of general responsiveness (not present before the trauma), as indicated by 3 or more of the following: efforts to avoid thoughts, feelings, or conversations associated with the trauma; efforts to avoid activities, places, or people that arouse recollections of the trauma; inability to recall an important aspect of the trauma; significantly diminished interest or participation in significant activities; feeling of detachment or estrangement from others; restricted range of affect (e.g., unable to have loving feelings); sense of a foreshortened future (e.g., does not expect to have a career, marriage, children, or a normal life span); and persistent symptoms of increased arousal (not present before the trauma); or as indicated by 2 or more of the following: difficulty falling or staying asleep; irritability or outbursts of anger; difficulty concentrating; hypervigilance; and exaggerated startle response. The disturbance, which has lasted for at least a month, causes clinically significant distress or impairment in social, occupational, or other important arcas of functioning.

In certain embodiments, the compositions and methods disclosed herein prevent, reduce, eliminate or delay one or more of the symptoms including, but not limited to re-experiencing of the traumatic experience in the form of intrusive memories, nightmares, or flashbacks; emotional and physical reactions triggered by reminders of the trauma; distancing from others; decreased interest in activities and other people; numbing of feelings; avoidance of trauma reminders; hyperarousal symptoms, including disrupted sleep, irritability, hypervigilance, decreased concentration; increased startle reflex; and combinations thereof.

Whatever the source of the problem, some people with PTSD repeatedly relive the trauma in the form of nightmares and disturbing recollections during the day. They may also experience other sleep problems, feel detached or numb, or be easily startled. They may lose interest in things they used to enjoy and have trouble feeling affectionate. They may feel irritable, more aggressive than before, or even violent. Things that remind them of the trauma may be very distressing, which could lead them to avoid certain places or situations that bring back those memories. The disorder may be accompanied by depression, substance abuse, or one or more other anxiety disorders. In severe cases, the person may have trouble working or socializing.

### Major Depressive Disorder

Major depressive disorder refers to a class of syndromes characterized by negative affect and repeated episodes of depression without any history of independent episodes of mood elevation and over-activity that fulfill the criteria of mania. The age of onset and the severity, duration, and frequency of the episodes of depression are all highly variable. The disorder may begin at any age. The symptoms of major depressive disorder typically develop over days to weeks. Prodromal symptoms include generalized anxiety, panic attacks, phobias or depressive symptoms and may occur during several months preceding the episode. Individual episodes also last between 3 and 12 months but recur less frequently. Most patients are asymptomatic between episodes, but a minority of patients may develop a persistent depression, mainly in old age.

Individual episodes of any severity are often precipitated by stressful life events. Common symptoms of a depressive episode include reduced concentration and attention; reduced self-esteem and self-confidence; ideas of guilt and unworthiness, ideas or acts of self-harm or suicide; disturbed sleep; and diminished appetite. In certain embodiments, a major depressive episode follows a psychosocial stressor, e.g., death of a loved one, marital separation, childbirth, or the end of an important relationship.

The lowered mood varies little from day to day and is often unresponsive to circumstances, yet may show a characteristic diurnal variation as the day goes on. As with manic episodes, the clinical presentation shows marked individual variations, and atypical presentations are particularly common in adolescence. In some cases, anxiety, distress, and motor agitation may be more prominent at times that the depression, and the mood change may also be masked by added features such as irritability, excessive consumption of alcohol, histrionic behavior, and exacerbation of pre-existing phobic or obsessional symptoms, or by hypochondria.

### Vasoactive Intestinal Peptide Receptor 2 (VPAC2) Agonists

VPAC2 is part of a larger family of receptors that bind the neuropeptides pituitary adenylate cyclase activating peptide (PACAP) or vasoactive intestinal peptide (VIP). This receptor family includes two other receptors, VPAC1 and PAC1, and activation of these receptors by either PACAP or VIP exerts a range of physiological effects on nervous, endocrine, cardiovascular, muscular, and immune systems.

Bay 55-9837 is a modified vasoactive intestinal peptide (VIP) analog and a selective VPAC2 agonist originally developed by Bayer Corporation a potential therapy for NIDDM. Bay 55-9837 is described in, e.g., PCT Application Publication No. WO/2001/023240 and is presently commercially available from multiple manufacturers. Bay 55-9837, and other VPAC2 agonists, have also been proposed for the treatment of spinal muscular atrophy, HIV/AIDS, and pulmonary hypertension. Unfortunately, the therapeutic potential of Bay 55-9837 is limited by its short in vivo half-life, as well as the possibility of increasing neuronal damage following ischemic injury. However, unexpectedly, the data provided herein demonstrate that a single low dose of Bay 55-9837 can exert a prophylactic effect against stress-induced maladaptive behaviors in subjects.

Ro 25-1553 and Ro 25-1392 are cyclic peptide VIP analogs described in, e.g., Yung et al., Jnl. Biol. Chem. 278(12): 10273-10281 (2003). Ro 25-1553 designed to overcome many of the deficiencies inherent in VIP and was initially investigated for use in treating bronchospastic diseases because of its rapid and long-lasting relaxation of tracheal and bronchial smooth muscles as well as a reduction of edema and cosinophilic mobilization during pulmonary anaphylaxis. Ro 25-1392 is less well-studied, but is a highly selective VPAC2 agonist.

Accordingly, the inventions described herein relate to VPAC2 agonists which prevent and protect against stress-induced maladaptive behaviors. In some embodiments, VPAC2 agonists may be delivered to a subject in the form of a pharmaceutical composition, which may comprise one or more pharmaceutically acceptable carriers, diluents, or excipients. Pharmaceutical compositions may be formulated as desired using art recognized techniques. Various pharmaceutically acceptable carriers, which include vehicles, adjuvants, and diluents, are readily available from numerous commercial sources. Moreover, an assortment of pharmaceutically acceptable auxiliary substances, such as pH adjusting and buffering agents, tonicity adjusting agents, stabilizers, wetting agents, and the like, are also available. Certain non-limiting exemplary carriers include saline, buffered saline, dextrose, water, glycerol, ethanol, and combinations thereof. Pharmaceutical compositions may be frozen and thawed prior to administration or may be reconstituted in WFI with or without additional additives. Bay 55-9837 is soluble to 2 mg/mL when reconstituted. VPAC2 agonists described herein are preferably formulated for oral or intravenous administration, but other routes of administration known in the art may be utilized.

VPAC2 agonists disclosed herein are intended to be administered to a subject in a prophylactically effective amount (i.e., an amount that prevents and protects against one or more symptoms of stress-induced maladaptive behaviors as described above). In certain embodiments, the prophylactically effective amount or dose may be adjusted depending on conditions of the disease/disorder to be treated or prophetically treated, the age, body weight, general health conditions, sex, and diet of the subject, dose intervals, administration routes, excretion rate, and combinations of drug.

In some embodiments, a prophylactically effective amount or dose of the VPAC2 agonist is about 0.5 mg/kg to about 15 mg mg/kg body weight of the subject to which the VPAC2 agonist is administered. In some embodiments, a prophylactically effective amount or dose of the VPAC2 agonist ranges from about 0.5 to about 14.5 mg/kg body weight, from about 0.5 to about 14.0 mg/kg body weight, from about 0.5 to about 13.5 mg/kg body weight, from about 0.5 to about 13.0 mg/kg body weight, from about 0.5 to about 12.5 mg/kg body weight, from about 0.5 to about 12.0 mg/kg body weight, from about 0.5 to about 11.5 mg/kg body weight, from about 0.5 to about 11.0 mg/kg body weight, from about 0.5 to about 10.5 mg/kg body weight, from about 0.5 to about 10.0 mg/kg body weight, from about 0.5 to about 9.5 mg/kg body weight, from about 0.5 to about 9.0 mg/kg body weight, from about 0.5 to about 8.5 mg/kg body weight, from about 0.5 to about 8.0 mg/kg body weight, from about 0.5 to about 7.5 mg/kg body weight, from about 0.5 to about 7.0 mg/kg body weight, from about 0.5 to about 6.5 mg/kg body weight, from about 0.5 to about 6.0 mg/kg body weight, from about 0.5 to about 5.5 mg/kg body weight, from about 0.5 to about 5.0 mg/kg body weight, from about 0.5 to about 4.5 mg/kg body weight, from about 0.5 to about 4.0 mg/kg body weight, from about 0.5 to about 3.5 mg/kg body weight, from about 0.5 to about 3.0 mg/kg body weight, from about 0.5 to about 2.5 mg/kg body weight, from about 0.5 to about 2.0 mg/kg body weight, from about 0.5 to about 1.5 mg/kg body weight, from about 0.5 to about 1.0 mg/kg body weight. In a preferred embodiment, the prophylactically effective amount or dose of the VPAC2 agonist is about 2.5 mg/kg to about 10 mg mg/kg body weight of the subject to which the VPAC2 agonist is administered.

In some embodiments, VPAC2 agonists may be administered to a subject in multiple doses as part of prophylactic regimen. An initial dose may be larger, followed by one or more smaller maintenance doses. Other ranges are possible, depending on the subject's response to a stressor. An initial dose may be the same as, or lower or higher than subsequently administered doses. VPAC2 agonists and compositions containing the same may be administered daily, weekly, biweekly, several times daily, semi-weekly, every other day, bi-weekly, quarterly, several times per week, semi-weekly, monthly, or more. The duration and frequency of administration may depend upon the subject's response to treatment.

In certain embodiments, when more than one dose of a VPAC2 agonist is administered to a subject, the second dose is lower than the first dose. In certain embodiments, the second dose is an amount that is at most one-half, one-quarter, or one-tenth the amount of the first dose. The number and frequency of doses may be determined based on the subject's response to administration of the VPAC2 agonist, e.g., if the subject tolerates administration of the composition without adverse reaction.

In certain embodiments, the VPAC2 agonist is administered to a subject prior to a stressor. In certain embodiments, the VPAC2 agonist is administered to a subject both prior to and after a stressor. In certain embodiments, the VPAC2 agonist is administered to a subject after a stressor. In certain embodiments, the VPAC2 agonist is administered to a subject prior to a stressor, and again prior to a recurrence of the stressor or a different stressor. In certain embodiments, the VPAC2 agonist is administered to the subject about 1 hour to about 5 hours, about 1 hour to about 1 day, about 5 hours to about 10 hours, about 10 hours to about 12 hours, 12 hours to about 1 day, 12 hours to about 4 weeks, about 18 hours to about 4 weeks, about 1 day to about 3.5 weeks, about 2 days to about 3 weeks, about 3 days to about 3 weeks, about 4 days to about 3 weeks, about 5 days to about 3 weeks, about 6 days to about 3 weeks, about 2 days to about 2.5 weeks, about 3 days to about 2.5 weeks, about 4 days to about 2.5 weeks, about 5 days to about 2.5 weeks, about 6 days to about 2.5 weeks, about 1 week to about 2.5 weeks, about 1 week to about 2.5 weeks, about 1 week to about 2 weeks, about 5 minutes to about 3 days, about 10 minutes to about 2 days, about 15 minutes to about 24 hours, about 20 minutes to about 12 hours, about 30 minutes to about 8 hours, about 45 minutes to about 5 hours, about 1 hour to about 12 hours, about 2 hours to about 5 hours, about 5 minutes, about 10 minutes, about 15 minutes, about 20 minutes, about 30 minutes, about 45 minutes, about 1 hour, about 2 hours, about 3 hours, about 4 hours, about 5 hours, about 6 hours, about 8 hours, about 10 hours, about 12 hours, about 15 hours, about 1 day, about 1.5 days, about 2 days, about 3 days, about 4 days, about 5 days, about 6 days, about 1 week, about 8 days, about 9 days, about 10 days, about 11 days, about 12 days, about 13 days, about 2 weeks, about 2.5 weeks, about 3 weeks, about 3.5 weeks, or about 4 weeks, prior to, and/or after a stressor.

### EXAMPLES

The following examples have been included to illustrate aspects of the inventions disclosed herein. In light of the present disclosure and the general level of skill in the art, those of skill appreciate that the following examples are intended to be exemplary only and that numerous changes, modifications, and alterations may be employed without departing from the scope of the disclosure.

### Example 1

### Effect of Bay 55-9837 on Stress in Female Mice

Saline, (R,S)-ketamine (10 mg/kg), or Bay 55-9837 (2.5 or 5 mg/kg) was administered 1 week prior to 3-shock contextual fear conditioning (CFC) stress in female 129S6/SvEv mice. Five days later, mice were assayed for fear, behavioral despair, locomotion, exploratory behavior, and hyponcophagia. Freezing was comparable across all groups during CFC training. Bay 55-9837 (2.5 mg/kg), but not (R,S)-ketamine (10 mg/kg) or Bay 55-9837 (5 mg/kg), significantly reduced freezing during CFC re-exposure when compared with saline.

On FST Day 1, immobility time was significantly reduced in mice administered Bay 55-9837 (2.5 mg/kg), but not (R,S)-ketamine (10 mg/kg) or Bay 55-9837 (5 mg/kg). On FST Day 2, both (R,S)-ketamine (10 mg/kg) and Bay 55-9837 (2.5 mg/kg), but not Bay 55-9837 (5 mg/kg), reduced behavioral despair. In mice administered 2.5 and 5 mg/kg of Bay 5-9837, but not (R,S)-ketamine (10 mg/kg), latency to feed in the novel environment was significantly lowered when compared to saline controls. Feeding in the home cage and weight loss following food deprivation was not significantly altered in experimental groups.

### Example 2

### Effect of Bay 55-9837 on Behavioral Despair and Hyponeophagia in Male Mice

Male 129S6/SvEv mice were administered the same behavioral paradigm as described in Example 1. Freezing during CFC training and re-exposure was comparable across all groups. There was no effect of drug on immobility time during FST Day 1. However, on FST Day 2, Bay 55-9837 (5 and 10 mg/kg), but not at 2.5 or 7.5 mg/kg, significantly reduced immobility when compared with saline controls.

Bay 55-9837 (7.5 mg/kg), but no other dose, suppressed latency to feed in the OF during the NSF assay. Latency to feed in the home cage was comparable across all groups. Mice administered Bay 55-9837 (7.5 mg/kg) lost significantly more body weight when compared to saline-administered mice.

### Example 3

Effect of Bay 55-9837 on Female Mice when Administered after Stress

Female 129S6/SvEv mice were given a single injection of saline, (R,S)-ketamine (10 mg/kg), or Bay 55-9837 (2.5 mg/kg) 5 minutes after 3-shock CFC training. Freezing during CFC training and re-exposure was not significantly altered by drug administration. Both (R,S)-ketamine and Bay 55-9837 significantly reduced immobility time on Day 2, but not Day 1, of the FST. Latency to feed in the open field was significantly decreased in mice administered Bay 55-9837, but not (R,S)-ketamine. Latency to feed in the home cage and body weight loss were comparable between all groups.

### Example 4

Effect of Bay 55-9837 on Male Mice when Administered after Stress

Male 129S6/SvEv mice were administered the same behavioral paradigm as in Example 3. (R,S)-ketamine and Bay 55-9837 administration did not significantly alter freezing during CFC training or re-exposure. As in female mice, both drugs tested significantly decreased immobility time on Day 2, but not Day 1, of the FST. Similarly, in the NSF, Bay 55-9837, but not (R,S)-ketamine suppressed latency to feed in the OF when compared with saline controls. Home cage feeding behavior and body weight loss were not significantly altered by drug administration.

### Example 5

### Effect of Bay 55-9837 on Stress-Induced Alterations in Sleep/Wake Cycles in Female Mice

Female 129S6/SEv mice were administered a single injection of saline of Bay 55-9837 (2.5 mg/kg) and placed in Piezo sleep boxes. One week later, mice were administered 3-shock CFC training or context exposure and again placed back into Piezo sleep boxes. Five days later, mice were re-exposed to the training context. On Day 2 after injection, there was no significant difference in activity between saline controls and Bay 55-9837-administered mice. Percentage of sleep and overall amplitude across Days 2-7 were comparable between saline and Bay 55-9837-administered mice. During CFC training, there was no effect of drug. Mice administered CFC froze significantly more on minute 5 when compared to mice given context exposure. During CFC re-exposure, mice exposed to CFC froze significantly more when compared with mice exposed to context exposure. In the CFC-administered group, mice given Bay 55-9837 exhibited significantly less freezing behavior compared to saline-administered mice. On Day 9, mice administered saline and exposed to CFC exhibited a significantly increased activity level at ZT12 when compared to non-stressed groups. Mice administered Bay 55-9837 prior to CFC had a significantly reduced activity level at ZT12 that was comparable to non-stressed mice. Percentage of sleep on days 9-11 was comparable across all groups. There was a significant effect of drug and stress as well as a drug x stress interaction. In the non-stressed mice, Bay 55-9837 did not significantly alter sleep/wake amplitude. Saline-administered mice exposed to CFC stress exhibited a significantly reduced amplitude when compared to non-stressed saline controls. Administration of Bay 55-9837 prior to stress significantly increase sleep/wake amplitude to a level comparable to non-stressed controls.

### Example 6

### Effect of Bay 55-9837 on Contextual Fear Discrimination in Mice

Mice were given a single injection of saline or Bay 55-9837 (2.5 mg/kg in female mice, 10 mg/kg in male mice) one week prior to contextual fear discrimination. Saline-administered female mice began discriminating on Day 7 whereas Bay 55-9837-administered mice began discriminating on Day 4. Bay 55-9837 significantly increased fear discrimination in female mice when compared to saline controls. By Day 5 of the behavioral paradigm, Bay 55-9837-administered, but not saline-administered, mice froze significantly less in Context B than in Context A. By Day 10, mice in both control and experimental groups discriminated between the two contexts. However, mice administered Bay 55-9837 exhibited a greater difference in freezing between Context A and Context B when compared to saline. Both saline and Bay 55-9837-administered male mice exhibited contextual fear discrimination. Saline mice began exhibiting fear discrimination on Day 5, while Bay 55-9837-administered mice began fear discriminating on Day 7. There was no significant difference in discrimination index between saline and Bay 55-9837-administered male mice. By Day 10, freezing in both contexts were comparable between both groups. In male mice, freezing in Context B was significantly lower than freezing in Context B in both saline and Bay 55-9837 groups.

### Example 7

### Fear Discrimination Learning Increases VPAC2 Receptor Expression

Mice were administered a contextual fear discrimination assay. Mice were sacrificed after Context B exposure either on Day 4 (high fear generalization) or on Day 10 (low fear generalization). Following euthanasia, brain-wide immunolabeling and imaging was used to assay VPAC2 expression throughout the brain. Mice sacrificed on Day 4 exhibited high levels of fear generalization. Freezing was not significantly different between Contexts A and B. Mice sacrificed on Day 11 exhibited low levels of fear generalization. By Day 11, freezing in Context B was significantly reduced when compared to freezing in Context A. Experimental protocol for brain-wide VPAC2 immunolabeling. Following Context B exposure, mice were sacrificed, and brains were harvested. Brains were serially sectioned, stained for VPAC2 expression using immunohistochemistry, and imaged. An automated cell counting pipeline was used to segment the cells, register brains to an atlas, and integrate cell counts for further analysis. Sample VPAC2 staining (left). Brains were automatically registered to a brain atlas (middle), and cell counts were integrated to obtain cell counts per brain region (right). Mice in the low fear generalization group exhibited significantly higher VPAC2 expression in the agranular insula area and CA3 of the hippocampus.

### Example 8

Bay 55-9837 Reduces Neural Activity in vCA3 During CFD Learning in Female Mice

Female mice were injected with GCaMP6f17 virus and a GRIN lens in vCA3 of the hippocampus, then base-plated following viral expression. Mice were then injected with saline or Bay 55-9837 (2.5 mg/kg) one week prior to administration of a CFD paradigm. Calcium imaging was performed during injection as well as Days 1, 2, 5, and 10 of the behavioral paradigm. As previously demonstrated, Bay 55-9837 facilitated and enhanced CFD in female mice when compared to saline controls. Neural activity was not significantly altered during injection of Bay 55-9837.

However, upon the first Context A exposure, Bay 55-9837 significantly reduced neural activity in vCA3. Directly after Context A learning, Bay 55-9837 significantly increased neural activity compared to saline controls. On Day 2 of CFD, Bay 55-9837 did not significantly alter neural activity in Context A, but did reduce activity in Context B, suggesting that Bay 55-9837 reduces neural activity during initial learning exposures in CFD. On Day 5 and Day 10 of CFD, Bay 55-9837 did not significantly alter neural activity compared to controls, suggesting that this suppression of activity is unique to the initial learning of the CFD contexts.

### Example 9

### VPAC2 Receptor Inhibition and Activation Occludes and Mimics the Prophylactic Effects of (R,S)-Ketamine in Female Mice.

Female 129S6/SvEv mice were administered an intracerebroventricular infusion of artificial cerebrospinal fluid, PACAP (6-38) (VPAC2 antagonist), or Bay 55-9837. Five minutes after, mice were given an IP injection of saline or (R,S)-ketamine (10 mg/kg). One week later, mice were administered 3-shock CFC training. Five days subsequently, mice were tested in CFC re-exposure and the FST. During CFC training, there was no significant difference in freezing across all groups. Upon CFC re-exposure, there was a significant group x drug interaction. Freezing was not significantly different between saline- and (R,S)-ketamine-administered mice in both the vehicle and PACAP(6-38) groups. Bay 55-9837/saline-administered mice exhibited significantly lower freezing when compared to both Vehicle/saline and Bay 55-9837/(R,S)-ketamine groups. Immobility time on FST Day 1 was comparable across all groups. On FST Day 2, Vehicle/(R,S)-ketamine-administered mice had significantly lower immobility when compared with Vehicle/saline-administered mice. In the PACAP (6-38) group, the prophylactic effect of (R,S)-ketamine was blocked. Bay 55-9837/saline-administered mice had significantly lower immobility when compared to the vehicle/saline group. However, in Bay 55-9837/(R,S)-ketamine-administered mice, immobility time was comparable to vehicle/saline controls.

### Example 10

Bay 55-9837 does not Alter Behavioral Despair in Non-Stressed Mice

Female and male 129S6/SvEv mice were given a single injection of saline, (R,S)-ketamine (10 mg/kg in females, 30 mg/kg in males), or Bay 55-9837 (2.5 mg/kg in females, 10 mg/kg in males) one hour prior to FST Day 1. FST Day 2 was administered 1 day later. In female mice, immobility time was comparable between all groups on both FST Day 1 and FST Day 2. Similarly, in male mice, drug administration did not significantly alter immobility time on either day of the FST.

### Example 11

### Bay 55-9837 Suppresses Hyponeophagia in Naïve Female Mice

Female 129S6/SvEv mice were administered a single injection of saline or Bay 55-9837 (2.5 mg/kg) one hour prior to the OF. On subsequent days, mice were administered the MB, EPM, and NSF assays. Locomotion and time spent in the center of the OF were comparable between both groups. Similarly, exploratory behavior in the EPM was not significantly altered by Bay 55-9837 administration. Bay 55-9837-administered mice buried a comparable amount of marbles as saline controls. Bay 55-9837 significantly reduced latency to feed in the OF during the NSF.

### Example 12

### Bay 55-9837 Reduces Perseverative Behavior and Suppresses Hyponeophagia in Male Mice.

Male 129S6/SvEv mice were administered the same behavioral paradigm as in Example 11. Distance travelled and time spent in the center of the OF was comparable between both groups. There was a trending, but not significant, increase in time spent in the open arms and center of the EPM. However, entries into the open arms and center were not significantly different between both groups. Similarly, there was a trending, but not significant, decrease in time spent in the closed arms. Entries into the closed arms was not significantly affected by drug administration. Bay 55-9837-administered mice buried significantly less marbles when compared to saline controls. Bay 55-9837-administered mice also exhibited a significantly reduced latency to feed in the OF during the NSF.

The disclosure of every patent, patent application, and publication cited herein is hereby incorporated herein by reference in its entirety.

While this invention has been disclosed with reference to particular embodiments, it is apparent that other embodiments and variations of this invention can be devised by others skilled in the art without departing from the true spirit and scope of the invention. The appended claims include all such embodiments and equivalent variations.

What is claimed is:

1. A method for preventing or delaying or treating a stress-induced affective disorder or stress-induced psychopathology in a subject, comprising administering to the subject a composition comprising a prophylactically effective amount of a vasoactive intestinal peptide receptor 2 (VPAC2) agonist, wherein administration of the composition prevents or delays at least one stress-induced maladaptive behavior in the subject selected from the group consisting of fear, depressive-like behavior, and anxiety-like behavior.

2. The method of claim 1, wherein administration of the composition prevents or delays multiple stress-induced maladaptive behaviors in the subject.

3. The method of claim 1, wherein the composition is administered to the subject prior to a stressor.

4. The method of claim 1, wherein the composition is administered to the subject after a stressor.

5. The method of claim 1, wherein the composition is administered to the subject at least once to the subject before a stressor and then after a stressor.

6. The method of claim 1, wherein the composition is administered to the subject orally, intravenously, intranasally, or via injection.

7. The method of claim 1, wherein the stress-induced affective disorder is selected from the group consisting of major depressive disorder and posttraumatic stress disorder (PTSD).

8. The method of claim 1, wherein the stress-induced affective disorder is selected from the group consisting of stress-induced psychopathology, depressive-like behavior and associated affective disorders, anhedonic behavior and associated affective disorders, anxiety and associated affective disorders, cognitive impairments and deficits and associated disorders, stress-induced fear, and combinations thereof.

9. The method of claim 1, wherein the VPAC2 agonist is selected from the group consisting of BAY 55-9837, Ro 25-1553, and Ro 25-1392.

10. The method of claim 9, wherein BAY 55-9837 is administered to the subject in an amount ranging from 2.5 mg/kg to 10 mg/kg.

11. A method for inducing and/or enhancing stress resilience in a subject, comprising administering to the subject a composition comprising a prophylactically effective amount of a vasoactive intestinal peptide receptor 2 (VPAC2) agonist, wherein administration of the composition induces and/or enhances stress resilience in the subject to at least one stress-induced maladaptive behavior selected from the group consisting of fear, depressive-like behavior, and anxiety-like behavior.

12. The method of claim 11, wherein administration of the composition induces and/or enhances stress resilience in the subject to multiple stress-induced maladaptive behaviors.

13. The method of claim 11, wherein the composition is administered to the subject prior to a stressor.

14. The method of claim 11, wherein the composition is administered to the subject after a stressor.

15. The method of claim 11, wherein the composition is administered to the subject at least once to the subject before a stressor and then after a stressor.

16. The method of claim 11, wherein the composition is administered to the subject orally, intravenously, intranasally, or via injection.

17. The method of claim 11, wherein the stress-induced affective disorder is selected from the group consisting of major depressive disorder and posttraumatic stress disorder (PTSD).

18. The method of claim 11, wherein the stress-induced affective disorder is selected from the group consisting of stress-induced psychopathology, depressive-like behavior and associated affective disorders, anhedonic behavior and associated affective disorders, anxiety and associated affective disorders, cognitive impairments and deficits and associated disorders, stress-induced fear, and combinations thereof.

19. The method of claim 11, wherein the VPAC2 agonist is selected from the group consisting of BAY 55-9837, Ro 25-1553, and Ro 25-1392.

20. The method of claim 19, wherein BAY 55-9837 is administered to the subject in an amount ranging from 2.5 mg/kg to 10 mg/kg.
